(19) United States
(12) Patent Application Publication
(10) Pub. No.: US 2011/0195979 A1
(43) Pub. Date: Aug. 11, 2011

(54) COMPOUNDS FOR THE TREATMENT AND PREVENTION OF INFLUENZA

(76) Inventors: Li Chen, Shanghai (CN); Xianfeng Lin, Shanghai (CN); Zongxing Qiu, Shanghai (CN); Guozhi Tang, Shanghai (CN); Lisha Wang, Shanghai (CN); Jim Zhen Wu, Shanghai (CN); Wengang Yang, Shanghai (CN)

(21) Appl. No.: 13/021,793

(22) Filed: Feb. 7, 2011

(30) Foreign Application Priority Data
Feb. 8, 2010 (CN) PCT/CN2010/070562

(51) Int. Cl. A61K 31/4965; C07C 215/70; C07C 255/58; C07C 237/30; C07C 311/39; C07C 69/157; C07D 213/74; C07D 239/42; C07D 239/47; C07D 239/84; A61K 31/136; A61K 31/166; A61K 31/18; A61K 31/277; A61K 31/222; A61K 31/4402; A61K 31/44; A61K 31/505; A61P 31/16

(52) U.S. Cl. 514/255.06; 564/443; 564/442; 558/418; 564/165; 564/440; 564/86; 558/419; 560/250; 546/312; 546/293; 544/332; 544/321; 544/292; 514/646; 514/620; 514/603; 514/524; 514/546; 514/352; 514/349; 514/275; 514/272

(57) ABSTRACT

A compound of formula (I)

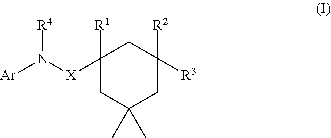

as well as pharmaceutically acceptable salt thereof, wherein R¹ to R⁴ and Ar are as defined in description and in claims, can be used as a medicament.

### CROSS-REFERENCE TO RELATED APPLICATIONS

This application claims the benefit of International Patent Application No. PCT/CN2010/070562, filed Feb. 8, 2010, which is hereby incorporated by reference in its entirety.

### FIELD OF THE INVENTION

The invention relates to compounds which are inhibitors of hemagglutinin (HA) and which are useful in the treatment or prevention of influenza.

Influenza viruses belong to the Orthomyxoviridae family of RNA viruses. Based on antigenic differences of viral nucleocapsid and matrix proteins, influenza viruses are further divided into three types named influenza A, B, and C viruses. All influenza viruses have an envelope, and their genomes are composed of eight or seven single-stranded, negative-sensed RNA segments. These viruses cause respiratory diseases in human and animals with a significant morbidity and mortality. Influenza pandemic of 1918, Spanish flu, is thought to have killed up to 100 million human beings. The reassortment of avian flu RNA fragments with circulating human viruses caused the other two pandemic, 1957 H2N2 “Asian influenza” and 1968 H3N2 “Hong Kong influenza”. Now, people around the world face the challenges of influenza from various angles: seasonal influenza epidemics affect about 5-15% of the world's population with an annual mortality ranging from 250,000 to 500,000. Infections of avian flu strains, mostly H5N1, have been reported in many Asian countries. Though no frequent human-to-human spreading was observed, avian flu infection was serious and associated with a high mortality of ˜60% of infected persons. More recently, an H1N1 swine flu infection appeared initially in North America. Its infection has already evolved into a new pandemic and more people will be affected by this virus in the coming seasons.

Currently, seasonal trivalent influenza vaccines and vaccines specific for H5N1 or swine flu are either available or in the phase of clinical trials. The prophylaxis is an effective method, at least in some populations, for preventing influenza virus infection and its potentially severe complications. However, due to continuous viral antigenicity shifting and drafting that makes future circulating flu strains unpredictable, and the limitations of massive production of vaccines within a relatively short period of time during a pandemic, other anti-flu approaches such as anti-flu drugs are highly desirable. On the market, there are two types of anti-flu drugs available: neuraminidase inhibitors such as oseltamivir phosphate (Tamilflu) and zanamivir (Relenza); and M2 ion channel blockers such as amantadine and rimantadine. To increase the effectiveness of current anti-flu drugs and prevent or attenuate appearance of drug-resistant viruses, it is invaluable to discover compounds with new mechanisms of anti-influenza actions, that can be used as a therapeutical or prophylactic agent alone or combined with current anti-flu drugs.

HA is a viral glycoprotein, located on the surface of virus particles. HA is synthesized as a precursor molecule, HA₀, and is cleaved by a cellular protease to yield two subunits, HA₁ and HA₂. This cleavage is indispensible for the function of HA. Individual HA₁ and HA₂, that are linked by one disulfide bond, assemble to form homotrimers on mature virus envelope. The life cycle of influenza virus infection begins with binding between the receptor binding pocket located in the membrane distal region of HA and sialic acid sugars on the surface of host epithelial cells. One of two types of sialic acids, N-acetylneuraminic acid α-(2,3)-Gal and N-acetylneuraminic acid α-(2,6)-Gal, is preferably recognized, dependent on host species involved. After HA-receptor binding, virus enters into cell by a process of endocytosis, resulting in the formation of virus-containing endosome. To release virus genomes into cytoplasm, fusion between viral envelope and endosome membrane occurs in an acidic environment within endosome that triggers an irreversibly conformational change of HA protein in which the hydrophobic fusion peptide at the N-terminus of HA2 is released from a buried position to a position that is 100 A away from its original location. The exposed fusogenic domain interacts with endosomal membrane and leads a series of structural rearrangements of HA2, which finally leads to fusion and release of viral RNP complexes into cytoplasm. Viral genomes then are translocated into nucleus where they act as the templates for virus RNA replication.

Since HA-mediated fusion is essential for influenza virus replication, HA has been used as a feasible target in the development of anti-influenza drugs. In general, there are two types of fusion inhibitors that block influenza infection by means of two different mechanisms: nonspecifically increasing pH in endosome or directly targeting at HA protein. The examples of the first mechanism include chloroquine (Ooi et al, 2006), triperiden (Oka et al, 2001), and maybe arbidol, a compound with a broad spectrum of anti-viral activities (Boriskin et al, 2006; Leneva et al, 2008). By interfering the process of H⁺ pumping into endosome, these compounds elevate energy barrier of endosome acidification. When intra-vesicle pH is higher than the critical point required for HA conformational change, fusion is blocked. For the second mechanism, small molecules bind and stabilize the pre-fusion structure of HA, resulting in inhibition at the step of viral membrane fusion. The following are several examples. BMY 27709 was discovered by a group of researchers at BMS Pharmaceutical Research Institute (Luo et al, 1997). This compound had a moderate anti-flu activity with an EC₅₀ of 6-8 μM for H1 and H2 viruses, but not H3 viruses. Resistant selection showed that resistant mutations were located around a position where HA₂ fusion peptide is hidden. Other mutations were scattered in both HA₂ and HA₁ regions. Studies of hemolysis and trypsin sensitivity assays suggested that HA itself was the target of the compound. Photoaffinity labeling indicated the presence of a binding pocket close to HA₂ N-terminal fusion peptide, but this site was not confirmed yet. Around the same time when BMS revealed BMY 27709, a group of scientists at Lilly Research Laboratories (Staschke et al, 1998) reported a novel HA inhibitor 180299, a podocarpic acid derivative. By isolating reassortment in co-infections with different viruses and mutation analysis, HA was assumed as the target of 180299. The study of pH-inactivation profiles of wild-type and some resistant variants and human erythrocyte fusion suggested that 180299 might bind to HA and stabilize its overall structure as BMY 27709 does. Another HA inhibitor, stachyflin, was found by Shionogi Discovery Research Laboratories (Yoshimoto et al, 1999). Based on mechanism of action studies, this compound showed similar profiles to BMY 27709. This compound only blocked H1 and H2 influenza virus replication, but not H3 viruses.

Besides library screening that was used to find several series of HA inhibitors described above, structure-based approach also joined in the journey and contributed to the discovery of HA inhibitors. A group of scientists at the University of California, San Francisco, using biostructure modeling and visual screen, found several HA fusion inhibitors and some of them were confirmed in both molecular based and cell based assays (Bodian et al, 1993; Hoffman et al, 1997). Most of those compounds had a behavior of an HA stabilizer but a compound named C22 might work through a different mechanism. The phenotype of resistants and the data of biochemical study suggested that this compound triggered a pre-matured conformational change of HA and irreversibly inactivated the virus. Based on this character, this type of compounds was named as HA destabilizer. Recently, a group of Chinese scientists also reported a series of thiazolidinone compounds and their analogs that showed fusion inhibition activities through destabilizing HA homotrimers (Yang and Luo, 2009).

Most recently, a specific binding site of tert-butyl hydroquinone (TBHQ) on HA₂ peptide of both H3 and H14 strains has been revealed in a co-crystallization study (Russell et al, 2008). This small molecule binds at a hydrophobic pocket located at an interface of two prefusion HA monomers. By means of an increase of the energy barrier required for HA fusogenic conformational changes in endosome low-pH environment, TBHQ inhibits fusion of H3 influenza viruses.

Taken together, though the extensive study of HA inhibitors has been carried out over 15 years, majority of these chemical series did not go beyond the stage of drug discovery except arbidol series that has been put into clinic in Russia.

### SUMMARY OF THE INVENTION

The application relates in particular to (i) a compound of formula (I)

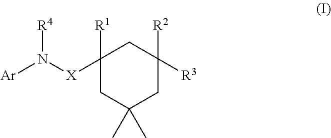

wherein
R¹ is hydrogen, C₁₋₆alkyl, or trifluoromethyl;
R²/R³ are hydrogen, halogen, OR¹⁰, or NR¹¹R¹²;
R⁴ is hydrogen, C₁₋₆alkyl, or trifluoromethyl;
X is —CH₂—, or carbonyl;
Ar is selected from

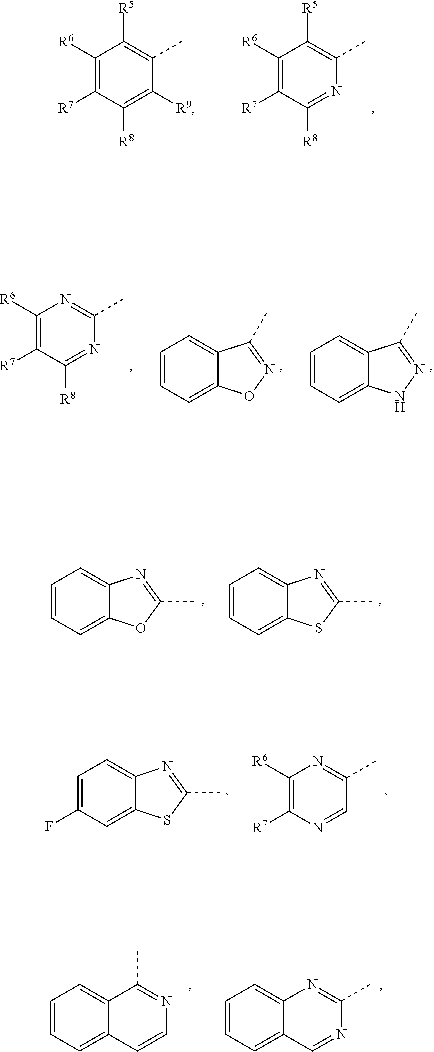

### Wherein

R⁵/R⁹ is hydrogen, halogen, trifluoromethyl, or C₁₋₆alkyl;
R⁶/R⁸ is hydrogen, halogen, trifluoromethyl, trifluoromethoxy, C₁₋₆alkoxy, cyano, C₁₋₆alkyl, —C(O)—NH₂, —S(O)₂—NH₂, or —S(O)₂—C₁₋₆alkyl;
R⁷ is hydrogen, halogen, C₁₋₆alkyl, cyano, C₁₋₆alkoxy, or —S(O)₂—NH₂;
R¹⁰ is hydrogen, C₁₋₆alkyl, carbonyl-C₁₋₆alkyl, or trifluoromethyl;
R¹¹ or R¹² is hydrogen, C₁₋₆alkyl, carbonyl-C₁₋₆alkyl, or sulfonyl;
provided that R¹, R², R³, R⁴, R⁵, R⁶, R⁷, R⁸ and R⁹ are not hydrogen simultaneously;
and pharmaceutically acceptable salt and stereoisomers thereof.

The invention also relates to a process for the manufacture of these novel compounds and medicaments containing them. These compounds are inhibitors of hemagglutinin (HA) and useful in the treatment or prevention of influenza.

### DETAILED DESCRIPTION OF THE INVENTION

### Definitions

As used herein, the term “C₁₋₆alkyl” alone or in combination signifies a saturated, linear- or branched chain alkyl group containing 1 to 6, preferably 1 to 4 carbon atoms, for example methyl, ethyl, propyl, isopropyl, 1-butyl, 2-butyl, tert-butyl and the like. Preferred “C₁₋₆alkyl” groups are methyl, ethyl, isopropyl, tert-butyl.

The term “C₁₋₆alkoxy” alone or in combination signifies a group C₁₋₆alkyl-O—, wherein the “C₁₋₆alkyl” is as defined above; for example methoxy, ethoxy, propoxy, isopropoxy, n-butoxy, i-butoxy, 2-butoxy, t-butoxy and the like. Preferred C₁₋₆alkoxy groups are methoxy and ethoxy and more preferably methoxy.

The term “halogen” means fluorine, chlorine, bromine or iodine. Halogen is preferably fluorine or chlorine.

The term “carbonyl” alone or in combination refers to the group —C(O)—.

The term “sulfonyl” alone or in combination refers to the group —S(O)₂—.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et. al., Organic Process Research & Development 2000, 4, 427-435; or in Ansel, H., et. al., In: Pharmaceutical Dosage Forms and Drug Delivery Systems, 6th ed. (1995), pp. 196 and 1456-1457. Preferred are the sodium salts of the compounds of formula (I).

“Pharmaceutically acceptable esters” means that compounds of general formula (I) may be derivatised at functional groups to provide derivatives which are capable of conversion back to the parent compounds in vivo. Examples of such compounds include physiologically acceptable and metabolically labile ester derivatives, such as methoxymethyl esters, methylthiomethyl esters and pivaloyloxymethyl esters. Additionally, any physiologically acceptable equivalents of the compounds of general formula (I), similar to the metabolically labile esters, which are capable of producing the parent compounds of general formula (I) in vivo, are within the scope of this invention. Preferred are the methyl and ethyl esters of the compounds of formula (I).

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “as defined above” and “as defined herein” when referring to a variable incorporates by reference the broad definition of the variable as well as preferred, more preferred and most preferred definitions, if any.

“Treating” or “treatment” of a disease state includes:

(i) preventing the disease state, i.e. causing the clinical symptoms of the disease state not to develop in a subject that may be exposed to or predisposed to the disease state, but does not yet experience or display symptoms of the disease state.
(ii) inhibiting the disease state, i.e., arresting the development of the disease state or its clinical symptoms, or
(iii) relieving the disease state, i.e., causing temporary or permanent regression of the disease state or its clinical symptoms.

Compounds of the general formula (I) which contain one or several chiral centers can either be present as racemates, diastereomeric mixtures, or optically active single isomers. The racemates can be separated according to known methods into the enantiomers. Preferably, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

All patents and publications identified herein are incorporated herein by reference in their entirety.

### Inhibitors of Hemagglutinin

The application provides a compound of formula (I)

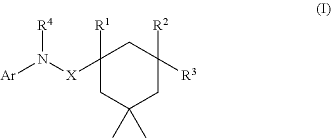

R¹ is hydrogen, C₁₋₆ alkyl, or trifluoromethyl;
R²/R³ are hydrogen, halogen, OR¹⁰, or NR¹¹R¹²
R⁴ is hydrogen, C₁₋₆ alkyl, or trifluoromethyl;
X is —CH₂—, or carbonyl;
Ar is selected from

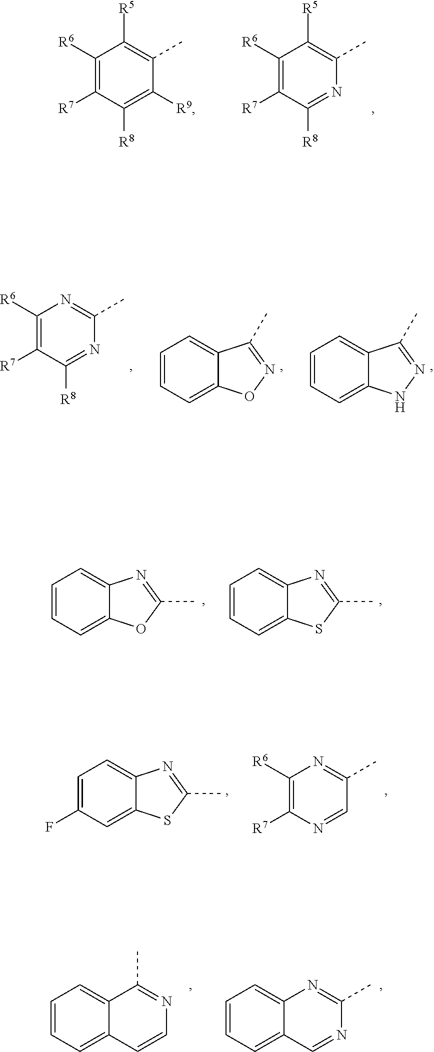

### Wherein

R⁵/R⁹ is hydrogen, halogen, trifluoromethyl, or C₁₋₆ alkyl;
R⁶/R⁸ is hydrogen, halogen, trifluoromethyl, trifluoromethoxy, C₁₋₆ alkoxy, cyano, C₁₋₆ alkyl, —C(O)—NH₂, —S(O)₂—NH₂, or —S(O)₂—C₁₋₆ alkyl;
R⁷ is hydrogen, halogen, C₁₋₆ alkyl, cyano, C₁₋₆ alkoxy, or —S(O)₂—NH₂
R¹⁰ is hydrogen, C₁₋₆ alkyl, carbonyl-C₁₋₆ alkyl, or trifluoromethyl;
R¹¹ or R¹² is hydrogen, C₁₋₆ alkyl, carbonyl-C₁₋₆ alkyl, or sulfonyl;
provided that R¹, R², R³, R⁴, R⁵, R⁶, R⁷, R⁸ and R⁹ are not hydrogen simultaneously; and pharmaceutically acceptable salt and stereoisomers thereof.

In one variation of the above compound,

R¹ is hydrogen or C₁₋₆ alkyl;
R²/R³ are hydrogen, halogen, OR¹⁰, or NR¹¹R¹²
R⁴ is hydrogen or C₁₋₆ alkyl;
X is —CH₂—, or carbonyl;
Ar is selected from

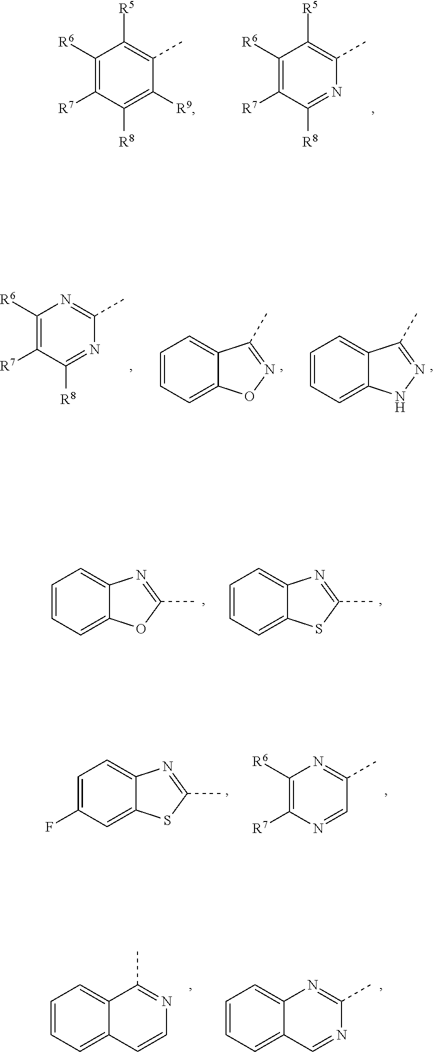

### Wherein

R⁵/R⁹ is hydrogen, halogen, trifluoromethyl, or C₁₋₆ alkyl;
R⁶/R⁸ is hydrogen, halogen, trifluoromethyl, trifluoromethoxy, C₁₋₆ alkoxy, cyano, C₁₋₆ alkyl, —C(O)—NH₂, —S(O)₂—NH₂, or —S(O)₂—C₁₋₆ alkyl;
R⁷ is hydrogen, halogen, C₁₋₆ alkyl, cyano, C₁₋₆ alkoxy, or —S(O)₂—NH₂
R¹⁰ is hydrogen, C₁₋₆ alkyl, carbonyl-C₁₋₆ alkyl, or trifluoromethyl;
R¹¹ or R¹² is hydrogen, C₁₋₆ alkyl, carbonyl-C₁₋₆ alkyl, or sulfonyl;
provided that R¹, R², R³, R⁴, R⁵, R⁶, R⁷, R⁸ and R⁹ are not hydrogen simultaneously and compound with two chiral center is in cis configuration.

In another variation of the above compound,

Ar is selected from

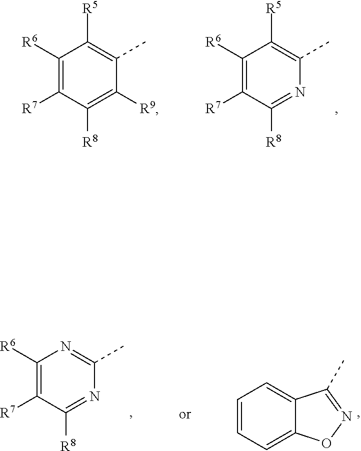

wherein R⁵/R⁹ is hydrogen or halogen;
R⁶/R⁸ is halogen, trifluoromethyl, trifluoromethoxy, cyano, —S(O)₂—NH₂, or —S(O)₂—C₁₋₆alkyl;
R⁷ is hydrogen, cyano, or halogen.

In one variation of any of the above compounds,

R⁴ is hydrogen.

In one variation of any of the above compounds,

R¹ is C₁₋₆ alkyl.

In one variation of any of the above compounds,

R¹ is methyl.

In one variation of any of the above compounds,

### R² or R³ is OR¹⁰.

In one variation of any of the above compounds,

R² or R³ is hydroxy.

In one variation of any of the above compounds,

R² or R³ is hydroxyl.

In one variation of any of the above compounds,

R² or R³ is hydroxyl and R⁴ is hydrogen.

In one variation of any of the above compounds,

### Ar is

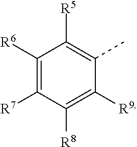

R⁵/R⁹ is hydrogen, chloro, or fluoro;
R⁶/R⁸ is halogen, trifluoromethyl, cyano, —S(O)₂—NH₂, or —S(O)₂-methyl;
R⁷ is hydrogen, chloro, or fluoro.

In one variation of any of the above compounds,

### X is —CH₂—.

In one variation of any of the above compounds,

R² or R³ is hydroxyl;
R⁴ is hydrogen;
Ar is selected from

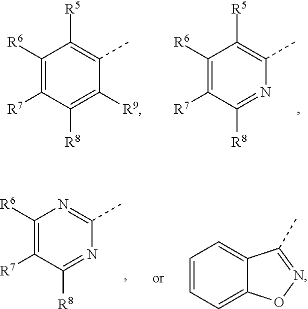

wherein R⁵/R⁹ is hydrogen or halogen;
R⁶/R⁸ is halogen, trifluoromethyl, trifluoromethoxy, cyano, —S(O)₂—NH₂, or —S(O)₂—C₁₋₆alkyl;
R⁷ is hydrogen, cyano, or halogen.

In one variation of any of the above compounds,

### Ar is

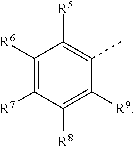

R⁵/R⁹ is hydrogen, chloro, or fluoro;
R⁶/R⁸ is halogen, trifluoromethyl, cyano, —S(O)₂—NH₂, or —S(O)₂-methyl;
R⁷ is hydrogen, chloro, or fluoro.

The application provides a compound selected from the group consisting of:
(cis-1,5)-3,3,5-Trimethyl-5-phenylaminomethyl-cyclohexanol,
(cis-1,3)-3-[(2-Chloro-phenylamino)-methyl]-3,5,5-trimethyl-cyclohexanol,
(cis-1,3)-3-[(3-Chloro-phenylamino)-methyl]-3,5,5-trimethyl-cyclohexanol,
(cis-1,3)-3-[(4-Chloro-phenylamino)-methyl]-3,5,5-trimethyl-cyclohexanol,
(cis-1,5)-3,3,5-Trimethyl-5-[(2-trifluoromethyl-phenylamino)-methyl]-cyclohexanol,
(cis-1,5)-3,3,5-Trimethyl-5-[(3-trifluoromethyl-phenylamino)-methyl]-cyclohexanol,
(cis-1,5)-3,3,5-Trimethyl-5-(p-tolylamino-methyl)-cyclohexanol,
(cis-1,3)-3-[(3-Methoxy-phenylamino)-methyl]-3,5,5-trimethyl-cyclohexanol,
3-[((cis-1,5)-5-Hydroxy-1,3,3-trimethyl-cyclohexylmethyl)-amino]-benzonitrile,
4-[((cis-1,5)-5-Hydroxy-1,3,3-trimethyl-cyclohexylmethyl)-amino]-benzonitrile,
(cis-1,3)-3-[(3-Isopropoxy-phenylamino)-methyl]-3,5,5-trimethyl-cyclohexanol,
(cis-1,3)-3-[(3-Isopropyl-phenylamino)-methyl]-3,5,5-trimethyl-cyclohexanol,
3-[((cis-1,5)-5-Hydroxy-1,3,3-trimethyl-cyclohexylmethyl)-amino]-benzamide,
(cis-1,3)-3-[(3-Methanesulfonyl-phenylamino)-methyl]-3,5,5-trimethyl-cyclohexanol,
3-[((cis-1,5)-5-Hydroxy-1,3,3-trimethyl-cyclohexylmethyl)-amino]-benzenesulfonamide,
(cis-1,3)-3-[(3-Chloro-5-fluoro-phenylamino)-methyl]-3,5,5-trimethyl-cyclohexanol,
(cis-1,3)-3-[(3-Fluoro-5-trifluoromethyl-phenylamino)-methyl]-3,5,5-trimethyl-cyclohexanol,
(cis-1,3)-3-[(3-Chloro-5-methyl-phenylamino)-methyl]-3,5,5-trimethyl-cyclohexanol,
(cis-1,3)-3-[(3,5-Dichloro-phenylamino)-methyl]-3,5,5-trimethyl-cyclohexanol,
(cis-1,3)-3-[(3-Chloro-5-trifluoromethyl-phenylamino)-methyl]-3,5,5-trimethyl-cyclohexanol,
(cis-1,3)-3-[(3-Bromo-5-trifluoromethyl-phenylamino)-methyl]-3,5,5-trimethyl-cyclohexanol,
3-Fluoro-5-[((cis-1,5)-5-hydroxy-1,3,3-trimethyl-cyclohexylmethyl)-amino]-benzonitrile,
3-Chloro-5-[((cis-1,5)-5-hydroxy-1,3,3-trimethyl-cyclohexylmethyl)-amino]-benzonitrile,
3-[((cis-1,5)-5-Hydroxy-1,3,3-trimethyl-cyclohexylmethyl)-amino]-5-trifluoromethyl-benzonitrile,
(cis-1,3)-3-[(3,5-Bis-trifluoromethyl-phenylamino)-methyl]-3,5,5-trimethyl-cyclohexanol,
(cis-1,3)-3-[(2,3-Dichloro-phenylamino)-methyl]-3,5,5-trimethyl-cyclohexanol,
(cis-1,3)-3-[(3,4-Dichloro-phenylamino)-methyl]-3,5,5-trimethyl-cyclohexanol,
2-Fluoro-5-[((cis-1,5)-5-hydroxy-1,3,3-trimethyl-cyclohexylmethyl)-amino]-benzonitrile,
2-Chloro-4-[((cis-1,5)-5-hydroxy-1,3,3-trimethyl-cyclohexylmethyl)-amino]-benzonitrile,
2-Chloro-5-[((cis-1,5)-5-hydroxy-1,3,3-trimethyl-cyclohexylmethyl)-amino]-benzonitrile,
4-[((cis-1,5)-5-Hydroxy-1,3,3-trimethyl-cyclohexylmethyl)-amino]-phthalonitrile,
(cis-1,3)-3-[(4-Chloro-3-trifluoromethyl-phenylamino)-methyl]-3,5,5-trimethyl-cyclohexanol,
4-[((cis-1,5)-5-Hydroxy-1,3,3-trimethyl-cyclohexylmethyl)-amino]-2-trifluoromethyl-benzonitrile,
2-Chloro-5-[((cis-1,5)-5-hydroxy-1,3,3-trimethyl-cyclohexylmethyl)-amino]-benzenesulfonamide,
3-Chloro-5-[((cis-1,5)-5-hydroxy-1,3,3-trimethyl-cyclohexylmethyl)-amino]-benzenesulfonamide,
3-[((cis-1,5)-5-Hydroxy-1,3,3-trimethyl-cyclohexylmethyl)-amino]-5-trifluoromethyl-benzenesulfonamide,
3-Fluoro-5-[((cis-1,5)-5-hydroxy-1,3,3-trimethyl-cyclohexylmethyl)-amino]-benzenesulfonamide,
5-[((cis-1,5)-5-Hydroxy-1,3,3-trimethyl-cyclohexylmethyl)-amino]-2-methyl-benzenesulfonamide,
5-[((cis-1,5)-5-Hydroxy-1,3,3-trimethyl-cyclohexylmethyl)-amino]-2-methoxy-benzenesulfonamide,
4-[((cis-1,5)-5-Hydroxy-1,3,3-trimethyl-cyclohexylmethyl)-amino]-2-trifluoromethoxy-benzenesulfonamide,
(cis-1,3)-3-{[(3-Chloro-phenyl)-methyl-amino]-methyl}-3,5,5-trimethyl-cyclohexanol,
(3-Fluoro-5-trifluoromethyl-phenyl)-(1,3,3-trimethyl-cyclohexylmethyl)-amine,
Acetic acid (cis-1,5)-3,3,5-trimethyl-5-phenylaminomethyl-cyclohexyl ester,
(3,3-Difluoro-1,5,5-trimethyl-cyclohexylmethyl)-(3-fluoro-5-trifluoromethyl-phenyl)-amine,
(3-Bromo-5-trifluoromethyl-phenyl)-(3,3-difluoro-1,5,5-trimethyl-cyclohexylmethyl)-amine,
(3-Fluoro-5-trifluoromethyl-phenyl)-((cis-1,5)-5-methoxy-1,3,3-trimethyl-cyclohexylmethyl)-amine,
(cis-1,3)-3-[(3-Fluoro-5-trifluoromethyl-phenylamino)-methyl]-1,3,5,5-tetramethyl-cyclohexanol,
((cis-1,5)-5-Dimethylamino-1,3,3-trimethyl-cyclohexylmethyl)-(3-fluoro-5-trifluoromethyl-phenyl)-amine,
(cis-1,5)-5-[(3-Fluoro-5-trifluoromethyl-phenylamino)-methyl]-3,3-dimethyl-cyclohexanol,
(trans-1,5)-5-[(3-fluoro-5-trifluoromethyl-phenylamino)-methyl]-3,3-dimethyl-cyclohexanol,
(cis-1,5)-3,3,5-Trimethyl-5-(pyridin-2-ylaminomethyl)-cyclohexanol,
(cis-1,3)-3-[(5-Bromo-pyridin-2-ylamino)-methyl]-3,5,5-trimethyl-cyclohexanol,
(cis-1,5)-3,3,5-Trimethyl-5-[(6-trifluoromethyl-pyridin-2-ylamino)-methyl]-cyclohexanol,
(cis-1,3)-3-[(4-Chloro-pyridin-2-ylamino)-methyl]-3,5,5-trimethyl-cyclohexanol,
(cis-1,5)-3,3,5-Trimethyl-5-[(3-trifluoromethyl-pyridin-2-ylamino)-methyl]-cyclohexanol,
(cis-1,5)-3,3,5-Trimethyl-5-[(5-trifluoromethyl-pyridin-2-ylamino)-methyl]-cyclohexanol,
6-[((cis-1,5)-5-Hydroxy-1,3,3-trimethyl-cyclohexylmethyl)-amino]-pyridine-2-sulfonic acid amide,
2-[((cis-1,5)-5-Hydroxy-1,3,3-trimethyl-cyclohexylmethyl)-amino]-pyridine-4-sulfonic acid amide,
(cis-1,5)-3,3,5-Trimethyl-5-[(4-methyl-pyrimidin-2-ylamino)-methyl]-cyclohexanol,
(cis-1,5)-3,3,5-Trimethyl-5-[(4-trifluoromethyl-pyrimidin-2-ylamino)-methyl]-cyclohexanol,
(cis-1,3)-3-[(4-Methoxy-pyrimidin-2-ylamino)-methyl]-3,5,5-trimethyl-cyclohexanol,
(cis-1,3)-3-[(4,6-Dimethoxy-pyrimidin-2-ylamino)-methyl]-3,5,5-trimethyl-cyclohexanol,
(cis-1,3)-3-[(4,6-Dimethyl-pyrimidin-2-ylamino)-methyl]-3,5,5-trimethyl-cyclohexanol,
(cis-1,3)-3-[(4-Chloro-5-methoxy-pyrimidin-2-ylamino)-methyl]-3,5,5-trimethyl-cyclohexanol,
(cis-1,3)-3-[(6-Chloro-pyrazin-2-ylamino)-methyl]-3,5,5-trimethyl-cyclohexanol,
(cis-1,5)-3,3,5-Trimethyl-5-[(6-methyl-pyrazin-2-ylamino)-methyl]-cyclohexanol,
(cis-1,3)-3-[(6-Methoxy-pyrazin-2-ylamino)-methyl]-3,5,5-trimethyl-cyclohexanol,
(cis-1,3)-3-(Benzoxazol-2-ylaminomethyl)-3,5,5-trimethyl-cyclohexanol,
Benzoxazol-2-yl-(3,3-difluoro-1,5,5-trimethyl-cyclohexylmethyl)-amine,
Benzoxazol-2-yl-((cis-1,5)-5-methoxy-1,3,3-trimethyl-cyclohexylmethyl)-amine,
(cis-1,3)-3-(Benzothiazol-2-ylaminomethyl)-3,5,5-trimethyl-cyclohexanol,
(cis-1,3)-3-[(6-Fluoro-benzothiazol-2-ylamino)-methyl]-3,5,5-trimethyl-cyclohexanol,
(cis-1,3)-3-(Benzo[d]isoxazol-3-ylaminomethyl)-3,5,5-trimethyl-cyclohexanol,
(cis-1,3)-3-[(1H-Indazol-3-ylamino)-methyl]-3,5,5-trimethyl-cyclohexanol,
(cis-1,5)-3,3,5-Trimethyl-5-(quinazolin-2-ylaminomethyl)-cyclohexanol,
(cis-1,3)-3-(Isoquinolin-1-ylaminomethyl)-3,5,5-trimethyl-cyclohexanol,
(cis-1,5)-5-Hydroxy-1,3,3-trimethyl-cyclohexanecarboxylic acid phenylamide,
(cis-1,5)-5-Hydroxy-1,3,3-trimethyl-cyclohexanecarboxylic acid(4-fluoro-phenyl)-amide,
(cis-1,5)-5-Hydroxy-1,3,3-trimethyl-cyclohexanecarboxylic acid(3-chloro-phenyl)-amide,
(cis-1,5)-5-Hydroxy-1,3,3-trimethyl-cyclohexanecarboxylic acid(3-trifluoromethyl-phenyl)-amide,
(cis-1,5)-5-Hydroxy-1,3,3-trimethyl-cyclohexanecarboxylic acid(3-isopropyl-phenyl)-amide,
(cis-1,5)-5-Hydroxy-1,3,3-trimethyl-cyclohexanecarboxylic acid(3-tert-butyl-phenyl)-amide,
(cis-1,5)-5-Hydroxy-1,3,3-trimethyl-cyclohexanecarboxylic acid(3-ethoxy-phenyl)-amide,
(cis-1,5)-5-Hydroxy-1,3,3-trimethyl-cyclohexanecarboxylic acid(3-trifluoromethoxy-phenyl)-amide,
(cis-1,5)-5-Hydroxy-1,3,3-trimethyl-cyclohexanecarboxylic acid(3-methanesulfonyl-phenyl)-amide,
(cis-1,5)-5-Hydroxy-1,3,3-trimethyl-cyclohexanecarboxylic acid(3-sulfamoyl-phenyl)-amide,
(cis-1,5)-5-Hydroxy-1,3,3-trimethyl-cyclohexanecarboxylic acid(3,5-difluoro-phenyl)-amide,
(cis-1,5)-5-Hydroxy-1,3,3-trimethyl-cyclohexanecarboxylic acid(3-chloro-5-fluoro-phenyl)amide,
(cis-1,5)-5-Hydroxy-1,3,3-trimethyl-cyclohexanecarboxylic acid(3-fluoro-5-trifluoromethyl-phenyl)-amide,
(cis-1,5)-5-Hydroxy-1,3,3-trimethyl-cyclohexanecarboxylic acid(3-chloro-5-trifluoromethyl-phenyl)-amide,
(cis-1,5)-5-Hydroxy-1,3,3-trimethyl-cyclohexanecarboxylic acid(3-bromo-5-trifluoromethyl-phenyl)-amide,
(cis-1,5)-5-Hydroxy-1,3,3-trimethyl-cyclohexanecarboxylic acid(3-methoxy-5-trifluoromethyl-phenyl)-amide,
(cis-1,5)-5-Hydroxy-1,3,3-trimethyl-cyclohexanecarboxylic acid(3,5-dichloro-phenyl)-amide,
(cis-1,5)-5-Hydroxy-1,3,3-trimethyl-cyclohexanecarboxylic acid(3,5-dimethoxy-phenyl)-amide,
(cis-1,5)-5-Hydroxy-1,3,3-trimethyl-cyclohexanecarboxylic acid(4-chloro-3-fluoro-phenyl)amide,
(cis-1,5)-5-Hydroxy-1,3,3-trimethyl-cyclohexanecarboxylic acid(3,4-dichloro-phenyl)-amide,
(cis-1,5)-5-Hydroxy-1,3,3-trimethyl-cyclohexanecarboxylic acid(4-fluoro-3-trifluoromethyl-phenyl)-amide,
(cis-1,5)-5-Hydroxy-1,3,3-trimethyl-cyclohexanecarboxylic acid(4-chloro-3-trifluoromethyl-phenyl)-amide,
(cis-1,5)-5-Hydroxy-1,3,3-trimethyl-cyclohexanecarboxylic acid(4-methoxy-3-trifluoromethyl-phenyl)-amide,
(cis-1,5)-5-Hydroxy-1,3,3-trimethyl-cyclohexanecarboxylic acid(4-cyano-3-trifluoromethyl-phenyl)-amide,
(cis-1,5)-5-Amino-1,3,3-trimethyl-cyclohexanecarboxylic acid(3-chloro-phenyl)-amide,
(cis-1,5)-5-Acetylamino-1,3,3-trimethyl-cyclohexanecarboxylic acid(3-chloro-phenyl)-amide,
(cis-1,5)-Diacetylamino-1,3,3-trimethyl-cyclohexanecarboxylic acid(3-chloro-phenyl)-amide, and
(cis-1,5)-5-Methanesulfonylamino-1,3,3-trimethyl-cyclohexanecarboxylic acid(3-chloro-phenyl)-amide.

The application provides a compound selected from the group consisting of:
(cis-1,5)-3,3,5-Trimethyl-5-phenylaminomethyl-cyclohexanol,
(cis-1,3)-3-[(2-Chloro-phenylamino)-methyl]-3,5,5-trimethyl-cyclohexanol,
(cis-1,3)-3-[(3-Chloro-phenylamino)-methyl]-3,5,5-trimethyl-cyclohexanol,
(cis-1,3)-3-[(4-Chloro-phenylamino)-methyl]-3,5,5-trimethyl-cyclohexanol,
(cis-1,5)-3,3,5-Trimethyl-5-[(2-trifluoromethyl-phenylamino)-methyl]-cyclohexanol,
(cis-1,5)-3,3,5-Trimethyl-5-[(3-trifluoromethyl-phenylamino)-methyl]-cyclohexanol,
(cis-1,3)-3-[(3-Methoxy-phenylamino)-methyl]-3,5,5-trimethyl-cyclohexanol,
3-[((cis-1,5)-5-Hydroxy-1,3,3-trimethyl-cyclohexylmethyl)-amino]-benzonitrile,
(cis-1,3)-3-[(3-Isopropyl-phenylamino)-methyl]-3,5,5-trimethyl-cyclohexanol,
(cis-1,3)-3-[(3-Methanesulfonyl-phenylamino)-methyl]-3,5,5-trimethyl-cyclohexanol,
3-[((cis-1,5)-5-Hydroxy-1,3,3-trimethyl-cyclohexylmethyl)-amino]-benzenesulfonamide,
(cis-1,3)-3-[(3-Chloro-5-fluoro-phenylamino)-methyl]-3,5,5-trimethyl-cyclohexanol,
(cis-1,3)-3-[(3-Fluoro-5-trifluoromethyl-phenylamino)-methyl]-3,5,5-trimethyl-cyclohexanol,
(cis-1,3)-3-[(3-Chloro-5-methyl-phenylamino)-methyl]-3,5,5-trimethyl-cyclohexanol,
(cis-1,3)-3-[(3,5-Dichloro-phenylamino)-methyl]-3,5,5-trimethyl-cyclohexanol,
(cis-1,3)-3-[(3-Chloro-5-trifluoromethyl-phenylamino)-methyl]-3,5,5-trimethyl-cyclohexanol,
(cis-1,3)-3-[(3-Bromo-5-trifluoromethyl-phenylamino)-methyl]-3,5,5-trimethyl-cyclohexanol,
3-Fluoro-5-[((cis-1,5)-5-hydroxy-1,3,3-trimethyl-cyclohexylmethyl)-amino]-benzonitrile,
3-Chloro-5-[((cis-1,5)-5-hydroxy-1,3,3-trimethyl-cyclohexylmethyl)-amino]-benzonitrile,
3-[((cis-1,5)-5-Hydroxy-1,3,3-trimethyl-cyclohexylmethyl)-amino]-5-trifluoromethyl-benzonitrile,
(cis-1,3)-3-[(3,5-Bis-trifluoromethyl-phenylamino)-methyl]-3,5,5-trimethyl-cyclohexanol,
(cis-1,3)-3-[(3,4-Dichloro-phenylamino)-methyl]-3,5,5-trimethyl-cyclohexanol,
2-Fluoro-5-[((cis-1,5)-5-hydroxy-1,3,3-trimethyl-cyclohexylmethyl)-amino]-benzonitrile,
2-Chloro-4-[((cis-1,5)-5-hydroxy-1,3,3-trimethyl-cyclohexylmethyl)-amino]-benzonitrile,
2-Chloro-5-[((cis-1,5)-5-hydroxy-1,3,3-trimethyl-cyclohexylmethyl)-amino]-benzonitrile,
(cis-1,3)-3-[(4-Chloro-3-trifluoromethyl-phenylamino)-methyl]-3,5,5-trimethyl-cyclohexanol,
4-[((cis-1,5)-5-Hydroxy-1,3,3-trimethyl-cyclohexylmethyl)-amino]-2-trifluoromethyl-benzonitrile,
2-Chloro-5-[((cis-1,5)-5-hydroxy-1,3,3-trimethyl-cyclohexylmethyl)-amino]-benzenesulfonamide,
3-Chloro-5-[((cis-1,5)-5-hydroxy-1,3,3-trimethyl-cyclohexylmethyl)-amino]-benzenesulfonamide,
3-Fluoro-5-[((cis-1,5)-5-hydroxy-1,3,3-trimethyl-cyclohexylmethyl)-amino]-benzenesulfonamide,
(cis-1,3)-3-{[(3-Chloro-phenyl)-methyl-amino]-methyl}-3,5,5-trimethyl-cyclohexanol,
(3-Fluoro-5-trifluoromethyl-phenyl)-(1,3,3-trimethyl-cyclohexylmethyl)-amine,
Acetic acid (cis-1,5)-3,3,5-trimethyl-5-phenylaminomethyl-cyclohexyl ester,
(3,3-Difluoro-1,5,5-trimethyl-cyclohexylmethyl)-(3-fluoro-5-trifluoromethyl-phenyl)-amine,
(3-Fluoro-5-trifluoromethyl-phenyl)-((cis-1,5)-5-methoxy-1,3,3-trimethyl-cyclohexylmethyl)-amine,
(cis-1,3)-3-[(3-fluoro-5-trifluoromethyl-phenylamino)-methyl]-1,3,5,5-tetramethyl-cyclohexanol,
((cis-1,5)-5-Dimethylamino-1,3,3-trimethyl-cyclohexylmethyl)-(3-fluoro-5-trifluoromethyl-phenyl)-amine,
(cis-1,5)-5-[(3-fluoro-5-trifluoromethyl-phenylamino)-methyl]-3,3-dimethyl-cyclohexanol,
(trans-1,5)-5-[(3-fluoro-5-trifluoromethyl-phenylamino)-methyl]-3,3-dimethyl-cyclohexanol,
(cis-1,5)-3,3,5-trimethyl-5-[(6-trifluoromethyl-pyridin-2-ylamino)-methyl]-cyclohexanol,
(cis-1,5)-3,3,5-trimethyl-5-[(3-trifluoromethyl-pyridin-2-ylamino)-methyl]-cyclohexanol,
2-[((cis-1,5)-5-Hydroxy-1,3,3-trimethyl-cyclohexylmethyl)-amino]-pyridine-4-sulfonic acid amide,
(cis-1,5)-3,3,5-trimethyl-5-[(4-trifluoromethyl-pyrimidin-2-ylamino)-methyl]-cyclohexanol,
(cis-1,3)-3-[(4-methoxy-pyrimidin-2-ylamino)-methyl]-3,5,5-trimethyl-cyclohexanol,
(cis-1,3)-3-[(4,6-dimethyl-pyrimidin-2-ylamino)-methyl]-3,5,5-trimethyl-cyclohexanol,
(cis-1,3)-3-[(6-chloro-pyrazin-2-ylamino)-methyl]-3,5,5-trimethyl-cyclohexanol,
(cis-1,3)-3-(benzooxazol-2-ylaminomethyl)-3,5,5-trimethyl-cyclohexanol,
Benzooxazol-2-yl-(3,3-difluoro-1,5,5-trimethyl-cyclohexylmethyl)-amine,
Benzooxazol-2-yl-((cis-1,5)-5-methoxy-1,3,3-trimethyl-cyclohexylmethyl)-amine,
(cis-1,3)-3-(benzothiazol-2-ylaminomethyl)-3,5,5-trimethyl-cyclohexanol,
(cis-1,3)-3-[(6-fluoro-benzothiazol-2-ylamino)-methyl]-3,5,5-trimethyl-cyclohexanol,
(cis-1,3)-3-(benzo[d] isoxazol-3-ylaminomethyl)-3,5,5-trimethyl-cyclohexanol,
(cis-1,3)-3-[(1H-indazol-3-ylamino)-methyl]-3,5,5-trimethyl-cyclohexanol,
(cis-1,5)-3,3,5-trimethyl-5-(quinazolin-2-ylaminomethyl)-cyclohexanol,
(cis-1,3)-3-(Isoquinolin-1-ylaminomethyl)-3,5,5-trimethyl-cyclohexanol,
(cis-1,5)-5-Hydroxy-1,3,3-trimethyl-cyclohexanecarboxylic acid(3-chloro-phenyl)-amide,
(cis-1,5)-5-Hydroxy-1,3,3-trimethyl-cyclohexanecarboxylic acid(3-trifluoromethyl-phenyl)-amide,
(cis-1,5)-5-Hydroxy-1,3,3-trimethyl-cyclohexanecarboxylic acid(3-isopropyl-phenyl)-amide,
(cis-1,5)-5-Hydroxy-1,3,3-trimethyl-cyclohexanecarboxylic acid(3-trifluoromethoxy-phenyl)-amide,
(cis-1,5)-5-Hydroxy-1,3,3-trimethyl-cyclohexanecarboxylic acid(3-sulfamoyl-phenyl)-amide,
(cis-1,5)-5-Hydroxy-1,3,3-trimethyl-cyclohexanecarboxylic acid(3,5-difluoro-phenyl)-amide,
(cis-1,5)-5-Hydroxy-1,3,3-trimethyl-cyclohexanecarboxylic acid(3-chloro-5-fluoro-phenyl)amide,
(cis-1,5)-5-Hydroxy-1,3,3-trimethyl-cyclohexane carboxylic acid(3-fluoro-5-trifluoromethyl-phenyl)-amide,
(cis-1,5)-5-Hydroxy-1,3,3-trimethyl-cyclohexane carboxylic acid(3-chloro-5-trifluoromethyl-phenyl)-amide,
(cis-1,5)-5-Hydroxy-1,3,3-trimethyl-cyclohexanecarboxylic acid(3-bromo-5-trifluoromethyl-phenyl)-amide,
(cis-1,5)-5-Hydroxy-1,3,3-trimethyl-cyclohexanecarboxylic acid(3-methoxy-5-trifluoromethyl-phenyl)-amide,
(cis-1,5)-5-Hydroxy-1,3,3-trimethyl-cyclohexanecarboxylic acid(3,5-dichloro-phenyl)-amide,
(cis-1,5)-5-Hydroxy-1,3,3-trimethyl-cyclohexanecarboxylic acid(4-chloro-3-fluoro-phenyl)amide,
(cis-1,5)-5-Hydroxy-1,3,3-trimethyl-cyclohexanecarboxylic acid(3,4-dichloro-phenyl)-amide,
(cis-1,5)-5-Hydroxy-1,3,3-trimethyl-cyclohexane carboxylic acid(4-fluoro-3-trifluoromethyl-phenyl)-amide,
(cis-1,5)-5-Hydroxy-1,3,3-trimethyl-cyclohexane carboxylic acid(4-chloro-3-trifluoromethyl-phenyl)-amide,
(cis-1,5)-5-Acetylamino-1,3,3-trimethyl-cyclohexanecarboxylic acid(3-chloro-phenyl)-amide,
(cis-1,5)-Diacetylamino-1,3,3-trimethyl-cyclohexanecarboxylic acid(3-chloro-phenyl)-amide, and
(cis-1,5)-5-Methanesulfonylamino-1,3,3-trimethyl-cyclohexanecarboxylic acid(3-chloro-phenyl)-amide.

The application provides a method for treatment of influenza comprising administering to a subject in need thereof a therapeutically effective amount of any of the embodiments of formula I as described herein.

The application provides a method for prevention of influenza comprising administering to a subject in need thereof a therapeutically effective amount of any of the embodiments of formula I as described herein.

The application provides a method for the treatment or prevention of diseases that are related to HA inhibition comprising administering to a subject in need thereof a therapeutically effective amount of any of the embodiments of formula I as described herein.

The application provides a pharmaceutical composition comprising:
(a) a pharmaceutically acceptable carrier; and
(b) any of the embodiments of formula I as described herein.

The application provides any of the above compounds for use as a medicament.

The application provides any of the above compounds for the manufacture of a medicament for treatment or prevention of influenza.

The application provides a pharmaceutical composition comprising any of the above compounds and a therapeutically inert carrier.

The application provides a compound, pharmaceutical composition, method, or use as described herein.

The application provides a compound of formula (I) or a pharmaceutically acceptable salt thereof, wherein

R¹ is hydrogen or C₁₋₆ alkyl;
R²/R³ are hydrogen, halogen, OR¹⁰, or NR¹¹R¹²
R⁴ is hydrogen or C₁₋₆ alkyl;
X is —CH₂—, or carbonyl;
Ar is selected from

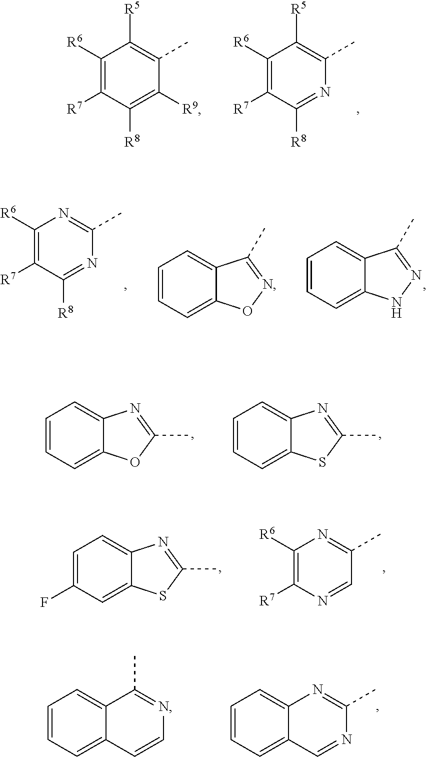

### Wherein

R⁵/R⁹ is hydrogen, halogen, trifluoromethyl, or C₁₋₆ alkyl;
R⁶/R⁸ is hydrogen, halogen, trifluoromethyl, trifluoromethoxy, C₁₋₆ alkoxy, cyano, C₁₋₆ alkyl, —C(O)—NH₂, —S(O)₂—NH₂, or —S(O)₂—C₁₋₆ alkyl;
R⁷ is hydrogen, halogen, C₁₋₆ alkyl, cyano, C₁₋₆ alkoxy, or —S(O)₂—NH₂
R¹⁰ is hydrogen, C₁₋₆ alkyl, carbonyl-C₁₋₆ alkyl, or trifluoromethyl;
R¹¹ or R¹² is hydrogen, C₁₋₆ alkyl, carbonyl-C₁₋₆ alkyl, or sulfonyl;
provided that R¹, R², R³, R⁴, R⁵, R⁶, R⁷, R⁸ and R⁹ are not hydrogen simultaneously, and compound with two chiral center is in cis configuration.

The application provides a compound of formula (I) or a pharmaceutically acceptable salt thereof, wherein R¹ is C₁₋₆ alkyl; preferably R¹ is methyl; R²/R³, R⁴, X, Ar, R⁵/R⁹, R⁶/R⁸, R⁷, R¹⁰, R¹¹ or R¹² is as defined in the above embodiments.

The application provides a compound of formula (I) or a pharmaceutically acceptable salt thereof, wherein R² or R³ is OR¹⁰; for example R² or R³ is hydroxyl.

The application provides a compound of formula (I) or a pharmaceutically acceptable salt thereof, wherein R⁴ is hydrogen; R¹, R²/R³, X, Ar, R⁵/R⁹, R⁶/R⁸, R⁷, R¹⁰, R¹¹ or R¹², is as defined in the above embodiments.

The application provides a compound of formula (I) or a pharmaceutically acceptable salt thereof, wherein R² or R³ is hydroxyl; R⁴ is hydrogen; R¹, X, Ar, R⁵/R⁹, R⁶/R⁸, R⁷, R¹⁰, R¹¹ or R¹² is as defined in the above embodiments.

The application provides a compound of formula (I) or a pharmaceutically acceptable salt thereof, wherein Ar is selected from

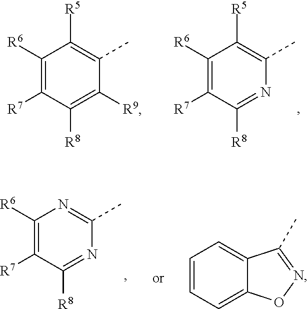

wherein R⁵/R⁹ is hydrogen or halogen;
R⁶/R⁸ is halogen, trifluoromethyl, trifluoromethoxy, cyano, —S(O)₂—NH₂, or —S(O)₂—C₁₋₆alkyl;
R⁷ is hydrogen, cyano, or halogen; R¹, R²/R³, X, R¹⁰, R¹¹ or R¹² is as defined in the above embodiments.

The application provides a compound of formula (I), wherein R² or R³ is hydroxyl and R⁴ is hydrogen.

The application provides a compound of formula (I) or a pharmaceutically acceptable salt thereof, wherein Ar is

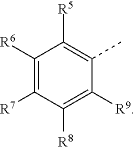

R⁵/R⁹ is hydrogen, chloro, or fluoro;
R⁶/R⁸ is halogen, trifluoromethyl, cyano, —S(O)₂—NH₂, or —S(O)₂-methyl;
R⁷ is hydrogen, chloro, or fluoro.

The application provides a compound of formula (I) or a pharmaceutically acceptable salt thereof, wherein X is —CH₂—; R¹, R²/R³, R⁴, Ar, R⁵/R⁹, R⁶/R⁸, R⁷, R¹⁰, R¹¹ or R¹² is as defined in the above embodiments.

The application provides a compound of formula (I) or a pharmaceutically acceptable salt thereof, wherein
R² or R³ is hydroxyl;
R⁴ is hydrogen;
Ar is selected from

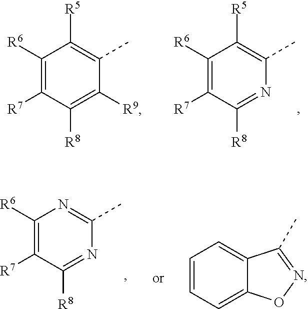

wherein R⁵/R⁹ is hydrogen or halogen;
R⁶/R⁸ is halogen, trifluoromethyl, trifluoromethoxy, cyano, —S(O)₂—NH₂, or —S(O)₂—C₁₋₆alkyl;
R⁷ is hydrogen, cyano, or halogen.

The application provides a compound of formula (I) or a pharmaceutically acceptable salt thereof, wherein

### Ar is

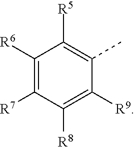

R⁵/R⁹ is hydrogen, chloro, or fluoro;
R⁶/R⁸ is halogen, trifluoromethyl, cyano, —S(O)₂—NH₂, or —S(O)₂-methyl;
R⁷ is hydrogen, chloro, or fluoro.

### Compounds

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOMTM v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE 1 depicts examples of compounds according to generic Formula I.

TABLE 1

| | | CPE_EC₅₀^{a} | Cytotoxicy |
|---|---|---|---|
| Ex. # | Compound Name | (μM) | IC₅₀^{b} (μM) |
| IIa-1 | (cis-1,5)-3,3,5-Trimethyl-5- | 0.43 | 37.36 |
| | phenylaminomethyl-cyclohexanol | | |
| IIa-2 | (cis-1,3)-3-[(2-Chloro-phenylamino)-methyl]- | 0.52 | 6.80 |
| | 3,5,5-trimethyl-cyclohexanol | | |
| IIa-3 | (cis-1,3)-3-[(3-Chloro-phenylamino)-methyl]- | 0.06 | 8.04 |
| | 3,5,5-trimethyl-cyclohexanol | | |
| IIa-4 | (cis-1,3)-3-[(4-Chloro-phenylamino)-methyl]- | 0.44 | 8.38 |
| | 3,5,5-trimethyl-cyclohexanol | | |
| IIa-5 | (cis-1,5)-3,3,5-Trimethyl-5-[(2- | 0.15 | 6.60 |
| | trifluoromethyl-phenylamino)-methyl]- | | |
| | cyclohexanol | | |
| IIa-6 | (cis-1,5)-3,3,5-Trimethyl-5-[(3- | 0.042 | 7.75 |
| | trifluoromethyl-phenylamino)-methyl]- | | |
| | cyclohexanol | | |
| IIa-8 | (cis-1,3)-3-[(3-Methoxy-phenylamino)- | 0.20 | >50.0 |
| | methyl]-3,5,5-trimethyl-cyclohexanol | | |
| IIa-9 | 3-[((cis-1,5)-5-Hydroxy-1,3,3-trimethyl- | 0.25 | 46.87 |
| | cyclohexylmethyl)-amino]-benzonitrile | | |
| IIa-12 | (cis-1,3)-3-[(3-Isopropyl-phenylamino)- | 0.090 | 6.68 |
| | methyl]-3,5,5-trimethyl-cyclohexanol | | |
| IIa-14 | (cis-1,3)-3-[(3-Methanesulfonyl- | 0.93 | >50 |
| | phenylamino)-methyl]-3,5,5-trimethyl- | | |
| | cyclohexanol | | |
| IIa-15 | 3-[((cis-1,5)-5-Hydroxy-1,3,3-trimethyl- | 0.28 | >50 |
| | cyclohexylmethyl)-amino]- | | |
| | benzenesulfonamide | | |
| IIa-16 | (cis-1,3)-3-[(3-Chloro-5-fluoro- | 0.017 | 4.40 |
| | phenylamino)-methyl]-3,5,5-trimethyl- | | |
| | cyclohexanol | | |
| IIa-17 | (cis-1,3)-3-[(3-Fluoro-5-trifluoromethyl- | 0.018 | 3.66 |
| | phenylamino)-methyl]-3,5,5-trimethyl- | | |
| | cyclohexanol | | |
| IIa-18 | (cis-1,3)-3-[(3-Chloro-5-methyl- | 0.025 | >3.16 |
| | phenylamino)-methyl]-3,5,5-trimethyl- | | |
| | cyclohexanol | | |
| IIa-19 | (cis-1,3)-3-[(3,5-Dichloro-phenylamino)- | 0.032 | 7.19 |
| | methyl]-3,5,5-trimethyl-cyclohexanol | | |
| IIa-20 | (cis-1,3)-3-[(3-Chloro-5-trifluoromethyl- | 0.044 | 6.70 |
| | phenylamino)-methyl]-3,5,5-trimethyl- | | |
| | cyclohexanol | | |
| IIa-21 | (cis-1,3)-3-[(3-Bromo-5-trifluoromethyl- | 0.058 | >3.16 |
| | phenylamino)-methyl]-3,5,5-trimethyl- | | |
| | cyclohexanol | | |
| IIa-22 | 3-Fluoro-5-[((cis-1,5)-5-hydroxy-1,3,3- | 0.050 | 23.89 |
| | trimethyl-cyclohexylmethyl)-amino]- | | |
| | benzonitrile | | |
| IIa-23 | 3-Chloro-5-[((cis-1,5)-5-hydroxy-1,3,3- | 0.034 | 13.87 |
| | trimethyl-cyclohexylmethyl)-amino]- | | |
| | benzonitrile | | |
| IIa-24 | 3-[((cis-1,5)-5-Hydroxy-1,3,3-trimethyl- | 0.040 | 13.17 |
| | cyclohexylmethyl)-amino]-5-trifluoromethyl- | | |
| | benzonitrile | | |
| IIa-25 | (cis-1,3)-3-[(3,5-Bis-trifluoromethyl- | 0.095 | 2.83 |
| | phenylamino)-methyl]-3,5,5-trimethyl- | | |
| | cyclohexanol | | |
| IIa-27 | (cis-1,3)-3-[(3,4-Dichloro-phenylamino)- | 0.068 | 2.49 |
| | methyl]-3,5,5-trimethyl-cyclohexanol | | |
| IIa-28 | 2-Fluoro-5-[((cis-1,5)-5-hydroxy-1,3,3- | 0.190 | 27.64 |
| | trimethyl-cyclohexylmethyl)-amino]- | | |
| | benzonitrile | | |
| IIa-29 | 2-Chloro-4-[((cis-1,5)-5-hydroxy-1,3,3- | 0.180 | 8.22 |
| | trimethyl-cyclohexylmethyl)-amino]- | | |
| | benzonitrile | | |
| IIa-30 | 2-Chloro-5-[((cis-1,5)-5-hydroxy-1,3,3- | 0.140 | 7.99 |
| | trimethyl-cyclohexylmethyl)-amino]- | | |
| | benzonitrile | | |
| IIa-32 | (cis-1,3)-3-[(4-Chloro-3-trifluoromethyl- | 0.018 | 2.75 |
| | phenylamino)-methyl]-3,5,5-trimethyl- | | |
| | cyclohexanol | | |
| IIa-33 | 4-[((cis-1,5)-5-Hydroxy-1,3,3-trimethyl- | 0.049 | 8.354 |
| | cyclohexylmethyl)-amino]-2-trifluoromethyl- | | |
| | benzonitrile | | |
| IIa-34 | 2-Chloro-5-[((cis-1,5)-5-hydroxy-1,3,3- | 0.086 | >50 |
| | trimethyl-cyclohexylmethyl)-amino]- | | |
| | benzenesulfonamide | | |
| IIa-35 | 3-Chloro-5-[((cis-1,5)-5-hydroxy-1,3,3- | 0.044 | 8.422 |
| | trimethyl-cyclohexylmethyl)-amino]- | | |
| | benzenesulfonamide | | |
| IIa-37 | 3-Fluoro-5-[((cis-1,5)-5-hydroxy-1,3,3- | 0.089 | 55 |
| | trimethyl-cyclohexylmethyl)-amino]- | | |
| | benzenesulfonamide | | |
| IIb-1 | (cis-1,3)-3-{[(3-Chloro-phenyl)-methyl- | 0.60 | 7.58 |
| | amino]-methyl}-3,5,5-trimethyl-cyclohexanol | | |
| IIc-1 | (3-Fluoro-5-trifluoromethyl-phenyl)-(1,3,3- | 0.237 | 2.32 |
| | trimethyl-cyclohexylmethyl)-amine | | |
| IId-1 | Acetic acid (cis-1,5)-3,3,5-trimethyl-5- | 0.492 | 64.44 |
| | phenylaminomethyl-cyclohexyl ester | | |
| IIe-1 | (3,3-Difluoro-1,5,5-trimethyl- | 0.264 | >3.16 |
| | cyclohexylmethyl)-(3-fluoro-5- | | |
| | trifluoromethyl-phenyl)-amine | | |
| IIf-1 | (3-Fluoro-5-trifluoromethyl-phenyl)-((cis- | 0.129 | >3.16 |
| | 1,5)-5-methoxy-1,3,3-trimethyl- | | |
| | cyclohexylmethyl)-amine | | |
| IIg-1 | (cis-1,3)-3-[(3-fluoro-5-trifluoromethyl- | 0.089 | >3.16 |
| | phenylamino)-methyl]-1,3,5,5-tetramethyl- | | |
| | cyclohexanol | | |
| IIh-1 | ((cis-1,5)-5-Dimethylamino-1,3,3-trimethyl- | 1.70 | 8.39 |
| | cyclohexylmethyl)-(3-fluoro-5- | | |
| | trifluoromethyl-phenyl)-amine | | |
| IIi-1/2 | Isomer 1: (cis-1,5)-5-[(3-fluoro-5- | <0.31 (Isomer 1), | 8.16 (Isomer 1), |
| | trifluoromethyl-phenylamino)-methyl]-3,3- | | |
| | dimethyl-cyclohexanol | | |
| | Isomer 2: (trans-1,5)-5-[(3-fluoro-5- | <0.31 (Isomer 2) | 16.8 (Isomer 2) |
| | trifluoromethyl-phenylamino)-methyl]-3,3- | | |
| | dimethyl-cyclohexanol | | |
| IIIa-3 | (cis-1,5)-3,3,5-trimethyl-5-[(6- | 0.27 | 13.46 |
| | trifluoromethyl-pyridin-2-ylamino)-methyl]- | | |
| | cyclohexanol | | |
| IIIa-5 | (cis-1,5)-3,3,5-trimethyl-5-[(3- | <0.15868 | 22.08 |
| | trifluoromethyl-pyridin-2-ylamino)-methyl]- | | |
| | cyclohexanol | | |
| IIIa-8 | 2-[((cis-1,5)-5-Hydroxy-1,3,3-trimethyl- | 0.98 | >100 |
| | cyclohexylmethyl)-amino]-pyridine-4- | | |
| | sulfonic acid amide | | |
| IIIa-10 | (cis-1,5)-3,3,5-trimethyl-5-[(4- | 0.12 | 47.82 |
| | trifluoromethyl-pyrimidin-2-ylamino)- | | |
| | methyl]-cyclohexanol | | |
| IIIa-11 | (cis-1,3)-3-[(4-methoxy-pyrimidin-2- | 1.50 | >50.00000 |
| | ylamino)-methyl]-3,5,5-trimethyl- | | |
| | cyclohexanol | | |
| IIIa-13 | (cis-1,3)-3-[(4,6-dimethyl-pyrimidin-2- | 1.37 | >100.00000 |
| | ylamino)-methyl]-3,5,5-trimethyl- | | |
| | cyclohexanol | | |
| IIIb-1 | (cis-1,3)-3-[(6-chloro-pyrazin-2-ylamino)- | 4.12 | >50.00000 |
| | methyl]-3,5,5-trimethyl-cyclohexanol | | |
| IIIc-1 | (cis-1,3)-3-(benzooxazol-2-ylaminomethyl)- | 1.42 | 83.00 |
| | 3,5,5-trimethyl-cyclohexanol | | |
| IIIc-2 | Benzooxazol-2-yl-(3,3-difluoro-1,5,5- | 3.36 | 10.87 |
| | trimethyl-cyclohexylmethyl)-amine | | |
| IIIc-3 | Benzooxazol-2-yl-((cis-1,5)-5-methoxy-1,3,3- | 7.43 | >50.00000 |
| | trimethyl-cyclohexylmethyl)-amine | | |
| IIIc-4 | (cis-1,3)-3-(benzothiazol-2-ylaminomethyl)- | 2.12 | 36.80 |
| | 3,5,5-trimethyl-cyclohexanol | | |
| IIIc-5 | (cis-1,3)-3-[(6-fluoro-benzothiazol-2- | 2.50 | 16.21 |
| | ylamino)-methyl]-3,5,5-trimethyl- | | |
| | cyclohexanol | | |
| IIId-1 | (cis-1,3)-3-(benzo[d]isoxazol-3- | 0.40 | >50 |
| | ylaminomethyl)-3,5,5-trimethyl-cyclohexanol | | |
| IIId-2 | (cis-1,3)-3-[(1H-indazol-3-ylamino)-methyl]- | 6.90 | >100 |
| | 3,5,5-trimethyl-cyclohexanol | | |
| IIIe-1 | (cis-1,5)-3,3,5-trimethyl-5-(quinazolin-2- | 8.51 | 26.72 |
| | ylaminomethyl)-cyclohexanol | | |
| IIIf-1 | (cis-1,3)-3-(Isoquinolin-1-ylaminomethyl)- | 2.37 | 43.74 |
| | 3,5,5-trimethyl-cyclohexanol | | |
| IVa-3 | (cis-1,5)-5-Hydroxy-1,3,3-trimethyl- | 0.460 | 82.07 |
| | cyclohexanecarboxylic acid (3-chloro- | | |
| | phenyl)-amide | | |
| IVa-4 | (cis-1,5)-5-Hydroxy-1,3,3-trimethyl- | 0.340 | 68.31 |
| | cyclohexanecarboxylic acid (3- | | |
| | trifluoromethyl-phenyl)-amide | | |
| IVa-5 | (cis-1,5)-5-Hydroxy-1,3,3-trimethyl- | 1.04 | 21.62 |
| | cyclohexanecarboxylic acid (3-isopropyl- | | |
| | phenyl)-amide | | |
| IVa-8 | (cis-1,5)-5-Hydroxy-1,3,3-trimethyl- | 0.861 | 60.03 |
| | cyclohexanecarboxylic acid (3- | | |
| | trifluoromethoxy-phenyl)-amide | | |
| IVa-10 | (cis-1,5)-5-Hydroxy-1,3,3-trimethyl- | 7.96 | >100 |
| | cyclohexanecarboxylic acid (3-sulfamoyl- | | |
| | phenyl)-amide | | |
| IVa-11 | (cis-1,5)-5-Hydroxy-1,3,3-trimethyl- | 0.320 | >100 |
| | cyclohexanecarboxylic acid (3,5-difluoro- | | |
| | phenyl)-amide | | |
| IVa-12 | (cis-1,5)-5-Hydroxy-1,3,3-trimethyl- | 0.055 | 56.43 |
| | cyclohexanecarboxylic acid (3-chloro-5- | | |
| | fluoro-phenyl)amide | | |
| IVa-13 | (cis-1,5)-5-Hydroxy-1,3,3-trimethyl- | 0.054 | 52.84 |
| | cyclohexanecarboxylic acid (3-fluoro-5- | | |
| | trifluoromethyl-phenyl)-amide | | |
| IVa-14 | (cis-1,5)-5-Hydroxy-1,3,3-trimethyl- | 0.019 | 22.16 |
| | cyclohexanecarboxylic acid (3-chloro-5- | | |
| | trifluoromethyl-phenyl)-amide | | |
| IVa-15 | (cis-1,5)-5-Hydroxy-1,3,3-trimethyl- | 0.055 | 7.31 |
| | cyclohexanecarboxylic acid (3-bromo-5- | | |
| | trifluoromethyl-phenyl)-amide | | |
| IVa-16 | (cis-1,5)-5-Hydroxy-1,3,3-trimethyl- | 0.060 | 74.77 |
| | cyclohexanecarboxylic acid (3-methoxy-5- | | |
| | trifluoromethyl-phenyl)-amide | | |
| IVa-17 | (cis-1,5)-5-Hydroxy-1,3,3-trimethyl- | 0.049 | 23.21 |
| | cyclohexanecarboxylic acid (3,5-dichloro- | | |
| | phenyl)-amide | | |
| IVa-19 | (cis-1,5)-5-Hydroxy-1,3,3-trimethyl- | 1.80 | 30.85 |
| | cyclohexanecarboxylic acid (4-chloro-3- | | |
| | fluoro-phenyl)amide | | |
| IVa-20 | (cis-1,5)-5-Hydroxy-1,3,3-trimethyl- | 0.627 | 9.63 |
| | cyclohexanecarboxylic acid (3,4-dichloro- | | |
| | phenyl)-amide | | |
| IVa-21 | (cis-1,5)-5-Hydroxy-1,3,3-trimethyl- | 0.454 | 29.59 |
| | cyclohexanecarboxylic acid (4-fluoro-3- | | |
| | trifluoromethyl-phenyl)-amide | | |
| IVa-22 | (cis-1,5)-5-Hydroxy-1,3,3-trimethyl- | 0.165 | 7.49 |
| | cyclohexanecarboxylic acid (4-chloro-3- | | |
| | trifluoromethyl-phenyl)-amide | | |
| IVb-2 | (cis-1,5)-5-Acetylamino-1,3,3-trimethyl- | 4.61 | >100 |
| | cyclohexanecarboxylic acid (3-chloro- | | |
| | phenyl)-amide | | |
| IVb-3 | (cis-1,5)-Diacetylamino-1,3,3-trimethyl- | 3.15 | 22.90 |
| | cyclohexanecarboxylic acid (3-chloro- | | |
| | phenyl)-amide | | |
| IVb-4 | (cis-1,5)-5-Methanesulfonylamino-1,3,3- | 2.77 | >100 |
| | trimethyl-cyclohexanecarboxylic acid (3- | | |
| | chloro-phenyl)-amide | | |

^{a}Compound concentration preventing virus infection caused CPE at a 50% level based OD measurement. Values are mean of triplicate experiments.
^{b}Half maximal cytotoxicity concentration of the compounds to MDCK cells.

### Synthesis: General Schemes.

### General Synthetic Route for Phenyl Based Analogues II (Scheme 1)

One category of the compounds described herein relates to (3,3-dimethyl-cyclohexylmethyl)phenylamines having the formula II wherein R¹ is hydrogen, methyl; R²/R³ is hydrogen, halogen, C₁₋₆alkoxy, or NR¹¹R¹²; and R⁴ is hydrogen:

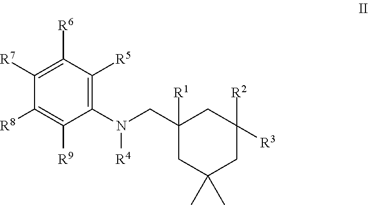

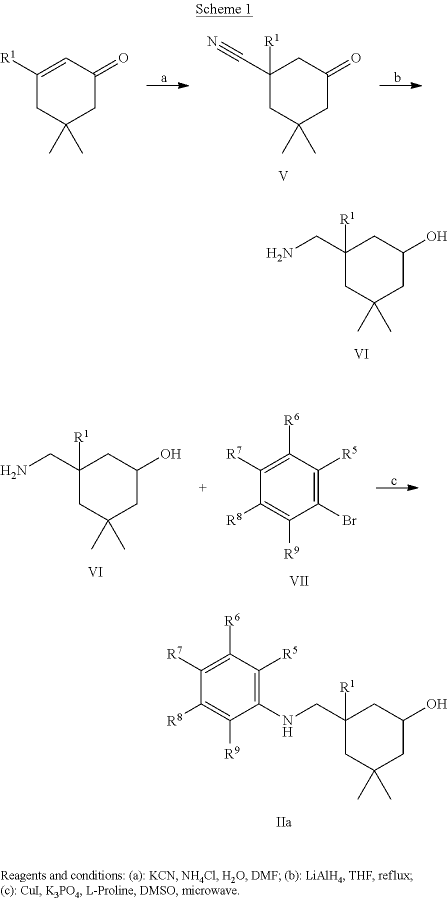

Compounds of interest IIa can be prepared according to the scheme above. 1-C₁₋₆alkyl-3,3-dimethyl-5-oxo-cyclohexanecarbonitrile V can be prepared by the Michael addition of cyanide to 3-C₁₋₆alkyl-5,5-dimethyl-cyclohex-2-enone. The reduction of Compound V by LiAlH₄ under refluxing conditions gives 3-aminomethyl-3-C₁₋₆alkyl-5,5-dimethyl-cyclohexanol VI. The target compounds IIa can be obtained by a copper-assisted Ullmann type cross coupling of amine VI with phenyl bromide VII.

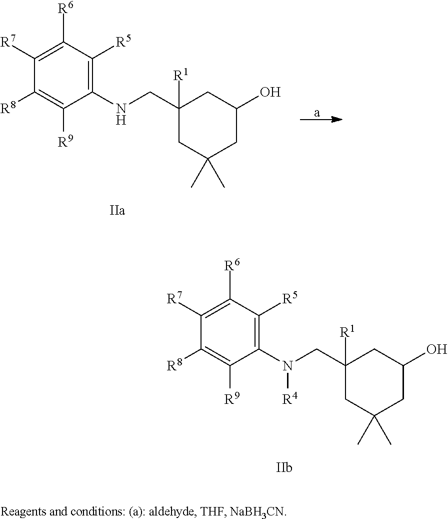

Compounds of interest IIb can be prepared by reductive alkylation of IIa with aldehyde and NaBH₃CN.

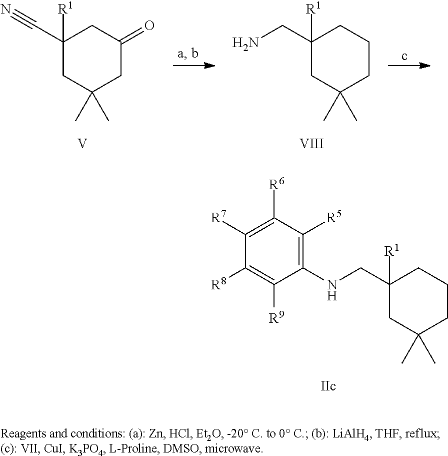

Compounds of interest IIc can be prepared by the method shown above. Following the reduction of ketone V by zinc in aqueous HCl, the so obtained cyano compound is refluxed with LiAlH₄ to give (3,3-dimethyl-cyclohexyl)-methylamine VIII. The copper catalyzed coupling of VIII and phenyl bromide VII affords IIc by microwave reactor.

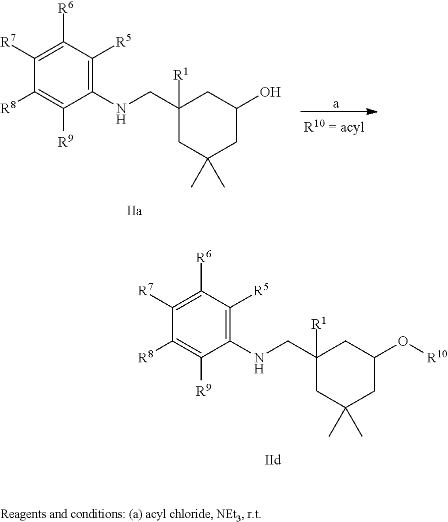

Compounds of interest IId can be prepared by acylation of IIa with acyl chloride and triethylamine.

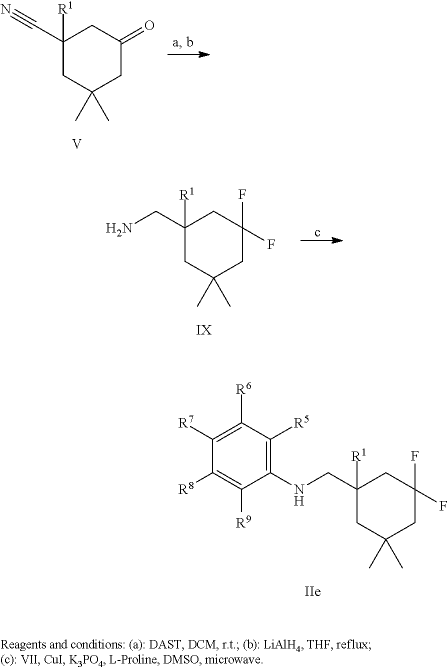

Compounds of interest IIe can be prepared through the synthesis route shown above. Following the conversion of ketone functional group to gem-difluoro, the so obtained cyano compound is refluxed with LiAlH₄ to give (3,3-difluoro-5,5-dimethyl-cyclohexyl)-methylamine IX. The copper catalyzed cross-coupling of IX and phenyl bromide VII gives target Compound IIe.

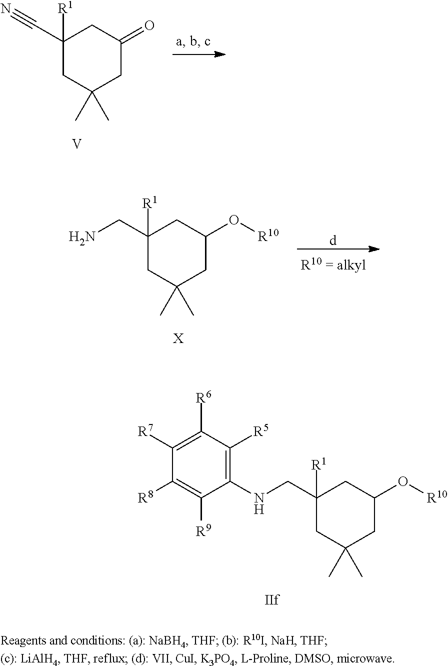

Compounds of interest IIf can be prepared by the method shown above. Following the reduction of the ketone functional group and alkylation of secondary alcohol, the ether intermediate can be reduced by LiAlH₄ to give amine X. Compound IIf is obtained by the copper catalyzed cross-coupling of amine X and phenyl bromide VII.

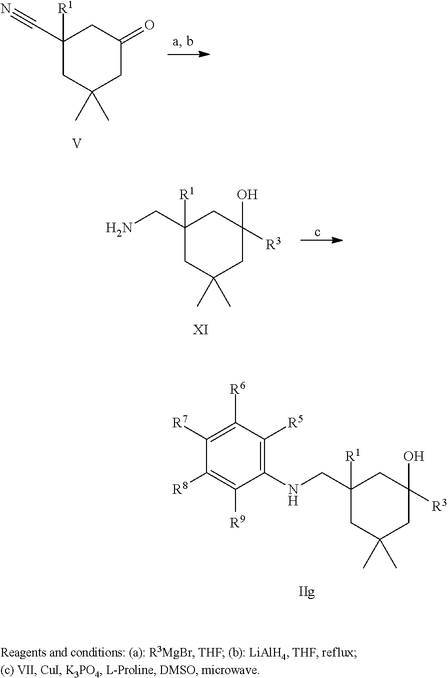

The tertiary alcohol IIg can be prepared by the method shown above. Following the addition reaction of Grignard reagent to the ketone functional group, the cyano intermediate is reduced by LiAlH₄ to give amine Compound XI, which is coupled with phenyl bromide VII to give target compound IIg.

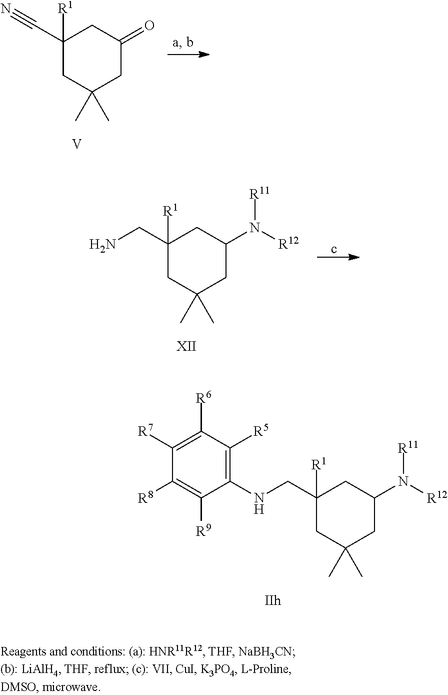

Compounds of interest IIh can be prepared by the method shown above. Following the reductive amination of the ketone functional group, the cyano intermediate is reduced by LiAlH₄ to give amine XII. Compounds IIh are prepared by the copper catalyzed cross-coupling of XII and phenyl bromide VII.

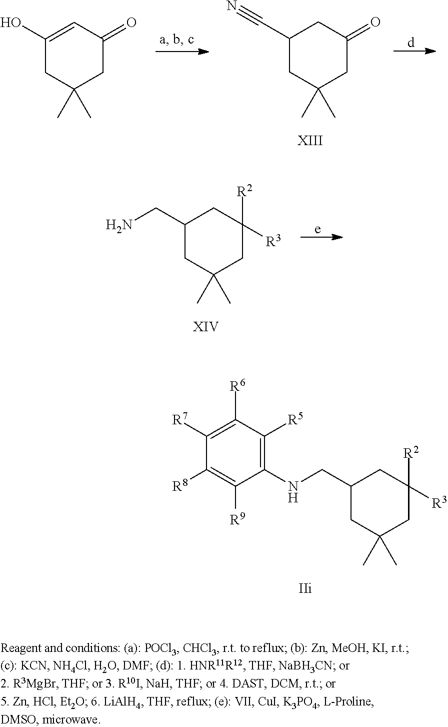

Compounds of interest IIi (R¹=H) can be prepared by the method shown above. 3-Hydroxy-5,5-dimethyl-cyclohex-2-enone is treated with POCl₃ to give 3-chloro-5,5-dimethyl-cyclohex-2-enone, which is reduced by zinc to afford 5,5-dimethyl-cyclohex-2-enone. After the addition of cyanide to the enone gave the cyano intermediate XIII, the ketone functional group can be derived to R²/R³ in a similar way as scaffolds IIc, and IIe, IIf, IIg and IIh. Compound XIII is reduced by LiAlH₄ to give amine XIV. Compounds of interest IIi can be obtained by the copper catalyzed cross-coupling of XIV and phenyl bromide VII.

Scheme 2: General Synthetic Route for Heterocycle Based Analogues with Formula III

One category of the compounds described herein relates to 3,3-dimethyl-cyclohexylmethylamines having the formula III (R²/R³=hydrogen, halogen, OR¹⁰, or NR¹¹R¹²). The heterocyclic aromatic amines are prepared according to general synthesis method as shown in the Scheme 2.

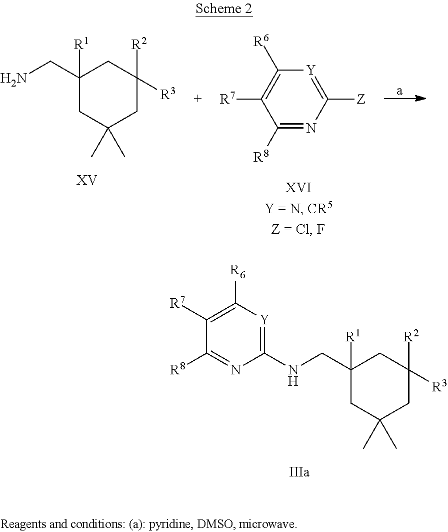

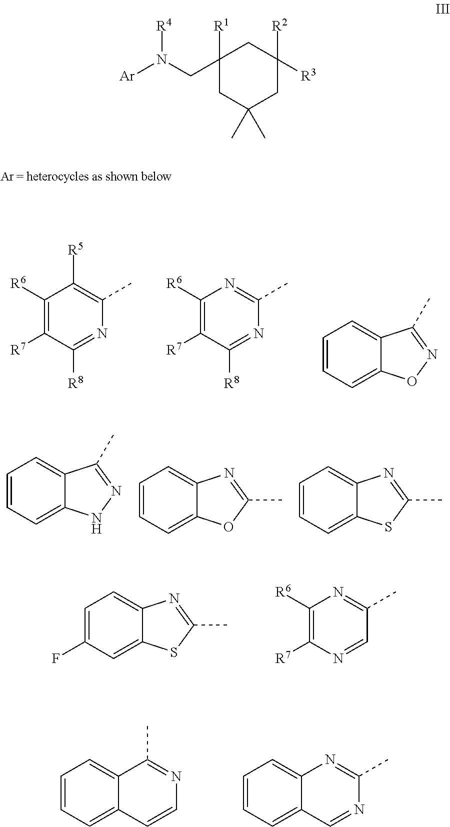

Compounds of interest IIIa can be prepared by the method shown above. The substituted 2-halogen pyrimidine or 2-halogen pyridine XVI is reacted with primary amine XV in microwave reactor to offer 2-aminopyrimidine (or pyridine) product IIIa. The primary amine XV can be prepared according to the methods shown in Scheme 1 and can be anyone of VI, VIII, IX, X, XI, XII, and XIV.

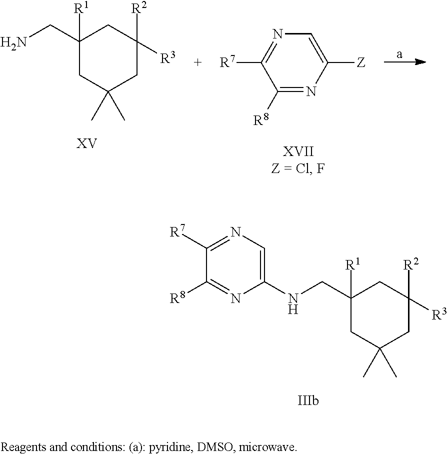

Compounds of interest IIIb can be prepared by the replacement reaction between substituted 2-halogen pyridazine XVII and primary amine XV. The amine XV is prepared according to the methods shown in Scheme 1 and can be anyone of VI, VIII, IX, X, XI, XII, and XIV.

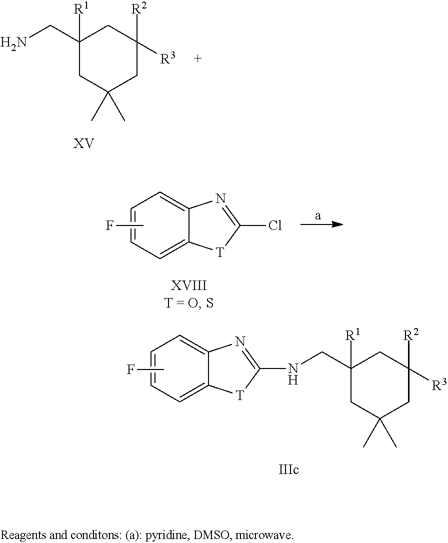

Compounds of interest IIIc can be prepared by the replacement reaction between substituted 2-chlorobenzothiazole (or 2-chlorobenzoxazole) XVIII and primary amine XV. As with other cases of IIIa and IIIb, the amine XV is prepared according to the methods shown in Scheme 1 and can be anyone of VI, VIII, IX, X, XI, XII, and XIV.

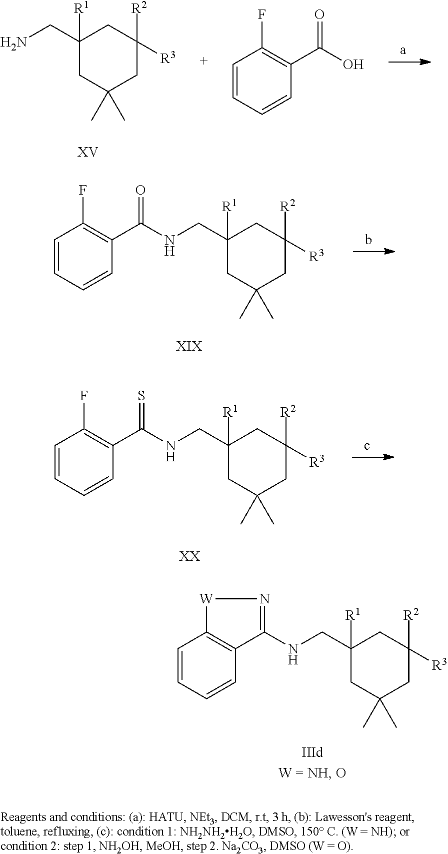

Compounds of interest IIId can be prepared by the synthesis method shown above. The primary amine XV is prepared according to the methods shown in Scheme 1 and can be anyone of VI, VIII, IX, X, XI, XII, and XIV. It can be coupled with 2-fluorobenzoic acid to give amide XIX. The thioamide XX is obtained by treating XIX with Lawesson's reagent. When thioamide XX is treated with hydrazine, it gives 1H-indazole target compound IIId. Whereas benzoisoxazole IIId can be prepared in two steps by the condensation of thioamide XX with hydroxylamine, and Na₂CO₃ treatment of the oxime product for benzoisoxazole ring formation.

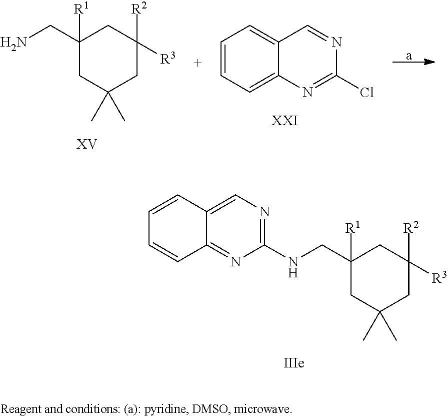

Compounds of interest IIIe can be prepared by the replacement reaction between substituted 2-chloroquinazoline XXI and primary amine XV. The amine XV is prepared according to the methods shown in Scheme 1 and can be anyone of VI, VIII, IX, X, XI, XII, and XIV.

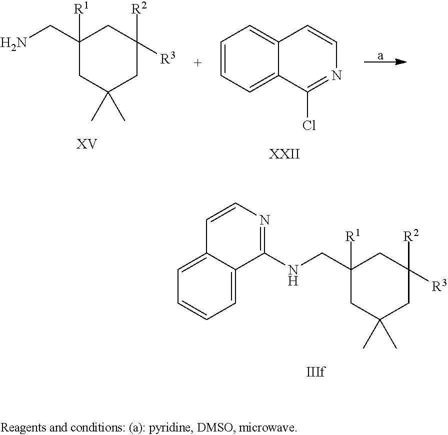

Compounds of interest IIIf can be prepared by the replacement reaction between substituted 2-chloroisoquinoline XXII and primary amine XV. The amine XV can be prepared according to the methods shown in Scheme 1 and can be anyone of VI, VIII, IX, X, XI, XII, and XIV.

### Scheme 3: General Synthetic Route for Amide-Based Analogues Iv

One category of the compounds described herein relates to 3,3-dimethyl-cyclohexanecarboxylic acid phenylamides having the formula IV (R²/R³=hydrogen, halogen, OR¹⁰, or NR¹¹R¹²). The heterocyclic aromatic amines are prepared according to general synthesis method as shown in the Scheme 3.

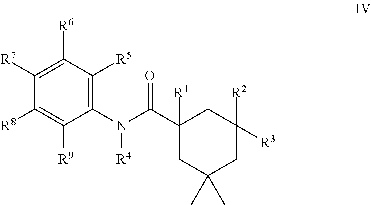

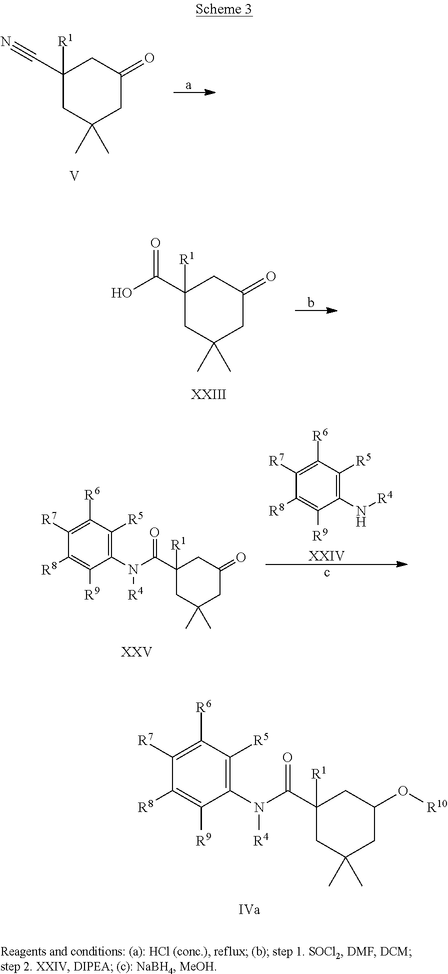

The preparation of amides with general structure IVa starts from 3,3-dimethyl-5-oxo-cyclohexanecarboxylic acid XXIV, which can be obtained through acidic hydrolysis of 3,3-dimethyl-5-oxo-cyclohexanecarbonitrile V by concentrated HCl. The acid XXIII is treated with thionyl chloride to give acyl chloride, which coupled with aniline XXIV to afford amide XXV. The reduction of the ketone functional group by NaBH₄ gives IVa, the alcohol functional group can be further derived by the methods shown in the Scheme 1 to give O—C₁₋₆alkyl analogs.

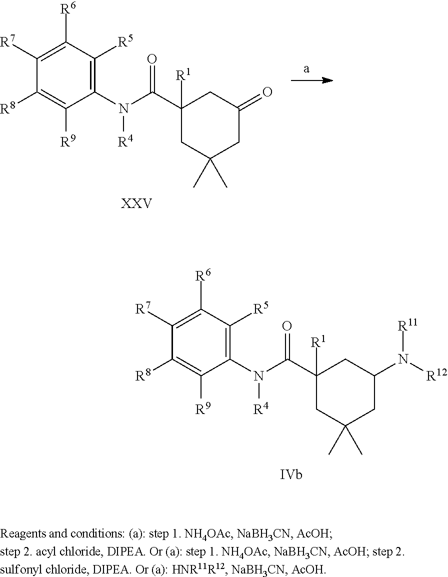

Compounds of interest IVb (R¹¹ or R¹² is hydrogen, C₁₋₆alkyl, carbonyl-C₁₋₆alkyl, or sulfonyl) can be prepared by the method shown above. The amino group can be incorporated by reductive amination of keto amide XXV. And the amino group is further derived by reaction with acyl chloride or sulfonyl chloride to give target compounds IVb.

### Pharmaceutical Compositions and Administration

The application provides a compound of formula (I) for use as therapeutically active substance.

The application provides a pharmaceutical composition comprising a compound of formula (I) and a therapeutically inert carrier.

The application provides the use of a compound of formula (I) for the preparation of medicaments useful in the treatment or prevention diseases that are related to HA inhibition is an object of the invention.

The application provides the use of a compound of formula (I) for the preparation of a medicament for the treatment or prevention of influenza.

Said medicaments, e.g. in the form of pharmaceutical preparations, can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions with an effective amount of a compound as defined above.

The above-mentioned pharmaceutical composition can be obtained by processing the compounds according to this invention with pharmaceutically inert inorganic or organic carriers. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical composition can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

The dosage depends on various factors such as manner of administration, species, age and/or individual state of health. The doses to be administered daily are about 5-400 mg/kg, preferably about 10-100 mg/kg, and can be taken singly or distributed over several administrations.

### Indications and Methods of Treatment

It has been found that the compounds of the present application belong to a new class of influenza inhibitors. In a one-cycle time-course study, these compounds acted at an early step of influenza virus replication. Results from HA-mediated hemolysis of chicken red blood cells and trypsin sensitivity of isolated HA in the presence of the compounds clearly showed that they targeted at HA. In cell-based assays involving multiple rounds of virus replication, the compounds inhibited an established influenza infection by dramatically reducing the production of progeny viruses by an order of more than 8-log when compared with a negative control. Considering current situations that people around the world are exposed to huge risks of infections of pandemic H1N1 swine flu, highly pathogenic H5N1 avian flu, and drug-resistant seasonal flu, it should be invaluable to study and develop HA inhibitors, including some compounds in this invention, for further clinical use.

The application provides a method for the treatment or prevention of diseases that are related to HA inhibition, which method comprises administering an effective amount of a compound of formula (I).

The application provides a method for the treatment or prevention of influenza, which method comprises administering an effective amount of a compound of formula (I).

### EXAMPLES

The compounds of the present application as well as their starting materials can be synthesized according to the following general reaction schemes 1 to 3. All substituents, in particular, R¹ to R¹², X, and Ar are as defined above unless otherwise indicated. Furthermore, and unless explicitly otherwise stated, all reactions, reaction conditions, abbreviations and symbols have the meanings well known to a person of ordinary skill in organic chemistry.

Abbreviations

bp: boiling point
DAST: diethylamino sulfur trifluoride
DIPEA: diisopropylethylamine
DCM: dichloromethylene
DMF: dimethylformamide
DMSO: dimethylsulfoxide
EDCI: 1-ethyl-3-(3-dimethylaminopropyl)carbodiimide
EtOAc: ethyl acetate
FBS: fetal bovine serum
g: gram
μg: microgram
EC₅₀: concentration required for 50% growth inhibition
IC₅₀: concentration required for 90% growth inhibition
h: hour
HATU: O-(7-azabenzotriazol-1-yl)-N,N,N′,N′-tetramethyluronium hexafluorophosphate
HOAc: acetic acid
HOBt: 1-hydroxybenzotriazole
HPLC: high performance liquid chromatography

### Hz: Hertz

MeOD: deuterated methanol
MeOH: methanol
mg: milligram
MHz: megahertz
mL: milliliter
mmol: millimole
MS (ESI): mass spectroscopy (electron spray ionization)
MW: molecular weight

### NMP: N-methylmorpholine

NMR: nuclear magnetic resonance
PET or Pet: petroleum ether
r.t.: room temperature
t-BuOK: potassium tert-butoxide
TEA: triethylamine
THF: tetrahydrofuran
TLC: thin layer chromatography
μL: microliter

The invention is illustrated by the following examples which have no limiting character. Unless explicitly otherwise stated, all reactions, reaction conditions, abbreviations and symbols have the meanings well known to a person of ordinary skill in organic chemistry.

### General Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μM; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 m, OBD™ 30×100 mm) column.

LC/MS spectra were obtained using a MicroMass Plateform LC (Waters™ alliance 2795-ZQ2000). Standard LC/MS conditions were as follows (running time 6 min):

Acidic condition: A: 0.1% formic acid in H₂O; B: 0.1% formic acid in acetonitrile;

Basic condition: A: 0.01% NH₃.H₂O in H₂O; B: acetonitrile;
Neutral condition: A: H₂O; B: acetonitrile.
Mass spectra (MS): generally only ions which indicate the parent mass are reported, and unless otherwise stated the mass ion quoted is the positive mass ion (M+H)⁺.
The microwave assisted reactions were carried out in a Biotage Initiator Sixty.
NMR Spectra were obtained using Bruker Avance 400 MHz.

All reactions involving air-sensitive reagents were performed under an argon atmosphere. Reagents were used as received from commercial suppliers without further purification unless otherwise noted.

The following examples were prepared by the general methods outlined in the schemes above. They are intended to illustrate the meaning of the present invention but should by no means represent a limitation within the meaning of the present invention:

### Preparative Examples

### Example IIa-1

### (cis-1,5)-3,3,5-Trimethyl-5-phenylaminomethyl-cyclohexanol

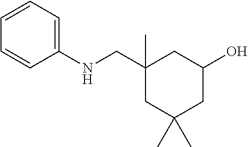

IIa-1 was prepared according to Synthetic route 1. The copper catalyzed cross coupling between bromide and amine was generally applied to the synthesis of other examples of IIa scaffold.

### Synthetic Route 1 to Example IIa-1

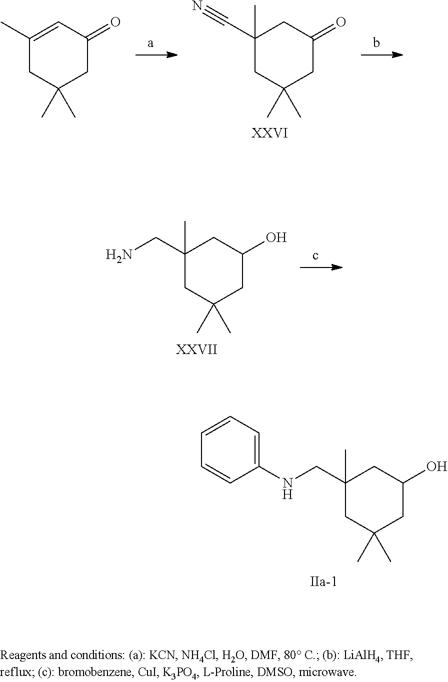

The Intermediate XXVI, 1,3,3-trimethyl-5-oxo-cyclohexanecarbonitrile, was made by the following procedure. To a stirring solution of isophorone (40 g, 0.289 mol) in 1.5 L of DMF was added NH₄Cl (24 g, 0.434 mol), KCN (54.4 g, 0.579 mol) and 200 mL of water. The mixture was stirred at 80° C. for 20 h. When the reaction was completed, the mixture was concentrated. The residue was dissolved into 300 mL of water and ethyl acetate. The aqueous phase was extracted by ethyl acetate. The combined organic phase was washed by water, dried over anhydrous Na₂SO₄, filtered and concentrated. The residue was purified by silica gel column (hexane:petroleum ether=50:1˜10:1) to give Compound XXVI (24 g, yield 50%). ¹H NMR (CDCl₃, 400 MHz), 2.64 (dt, 1H, J₁=14.4 Hz, J₂=2.0 Hz), 2.22 (dt, 1H, J₁=13.6 Hz, J₂=2.0 Hz), 2.16 (dd, 1H, J₁=12.0 Hz, J₂=1.2 Hz), 2.13 (m, 1H), 2.02 (dt, 1H, J₁=14.0 Hz, J₂=2.0 Hz), 1.58 (d, 1H, J=14.4 Hz), 1.46 (s, 3H), 1.15 (s, 3H), 1.04 (s, 3H).

The synthesis of Intermediate XXVII, 3-aminomethyl-3,5,5-trimethyl-cyclohexanol, was carried out by the following procedure. To a solution of Compound XXVI (10 g, 60 mmol) in THF (400 mL) was added LiAlH₄ (7 g, 0.18 mol) in portions. The mixture was refluxed for 16 h until no starting material remains (monitored by TLC, petroleum ether:ethyl acetate=5:1). After the mixture was cooled to 0° C., 20 mL of water was added dropwise under 0° C., followed by dropwise addition of 20 mL of NaOH (2N) at 0° C. The mixture was filtered and the filtrate was acidified by addition of aqueous HCl (2N). After washed by ethyl acetate, the aqueous phase was neutralized to pH>7 by addition of NaOH, and extracted by ethyl acetate and dichloromethylene. The combined organic layers were dried over Na₂SO₄, filtered and concentrated to give Compound XXVII (4 g, yield 39%). ¹H NMR (CDCl₃, 400 MHz), 3.97-3.91 (m, 1H), 2.38 (s, 2H), 1.74 (m, 1H), 1.64 (m, 1H), 1.16 (dt, 1H, J₁=14.0 Hz, J₂=2.0 Hz), 1.04-0.97 (m, 9H), 0.94 (s, 3H).

A mixture of bromobenzene (91 mg, 0.58 mmol), amine XXVII (150 mg, 0.88 mmol), K₃PO₄ (250 mg, 1.16 mmol), CuI (11 mg, 0.058 mmol), and L-proline (13 mg, 0.116 mmol) in 3 mL of DMSO was heated in microwave at 80° C. for 30 min. The reaction was monitored by LCMS (ESI). When the reaction was complete, it was send to HPLC separation without further work-up. After HPLC separation, the eluent was concentrated under vacuum to remove the organic solution. The residue was dried by lyophylization to give example IIa-1. ¹H NMR (d₄-MeOD, 400 MHz), 7.46 (t, 2H, J=7.6 Hz), 7.28 (d, 2H, J=8.0 Hz), 7.02 (t, 1H, J=7.6 Hz), 3.99-3.96 (m, 1H), 3.14 (s, 2H), 1.46-1.42 (m, 2H), 1.30-1.03 (m, 13H). MS (ESI): calc'd (M+H)⁺ 248.2, exp (M+H)⁺ 248.1.

### Example IIa-2

### (cis-1,3)-3-[(2-Chloro-phenylamino)-methyl]-3,5,5-trimethyl-cyclohexanol

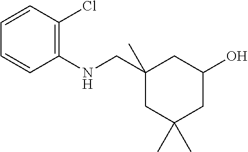

The title compound was prepared in analogy to example IIa-1 in Synthetic route 1 by using 2-chloro-bromobenzene instead of bromobenzene. ¹H NMR (d₄-MeOD, 400 MHz), 7.21 (d, 1H, J=8.0 Hz), 7.11 (t, 1H, J=8.0 Hz), 6.77 (d, 1H, J=8.0 Hz), 6.58 (t, 1H, J=8.0 Hz), 3.97-3.93 (m, 1H), 2.98 (s, 2H), 1.79-1.73 (m, 2H), 1.33-1.03 (m, 10H), 0.98 (s, 3H). MS (ESI): calc'd (M+H)⁺ 282.2, exp (M+H)⁺ 282.1.

### Example IIa-3

### (cis-1,3)-3-[(3-Chloro-phenylamino)-methyl]-3,5,5-trimethyl-cyclohexanol

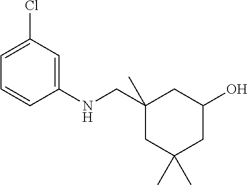

The title compound was prepared in analogy to example IIa-1 in Synthetic route 1 by using 3-chloro-bromobenzene instead of bromobenzene. ¹H NMR (d₄-MeOD, 400 MHz), 7.01 (t, 1H, J=8.0 Hz), 6.62 (s, 1H), 6.55 (d, 1H, J=8.0 Hz), 6.55 (d, 1H, J=8.0 Hz), 3.94-3.91 (m, 1H), 2.84 (s, 2H), 1.75-1.71 (m, 2H), 1.31-1.03 (m, 10H), 0.98 (s, 3H). MS (ESI): calc'd (M+H)⁺ 282.2, exp (M+H)⁺ 282.1.

### Example IIa-4

### (cis-1,3)-3-[(4-Chloro-phenylamino)-methyl]-3,5,5-trimethyl-cyclohexanol

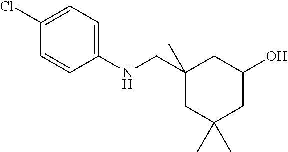

The title compound was prepared in analogy to example IIa-1 in Synthetic route 1 by using 4-chloro-bromobenzene instead of bromobenzene. ¹H NMR (d₄-MeOD, 400 MHz), 7.03 (d, 2H, J=8.4 Hz), 6.61 (d, 2H, J=8.4 Hz), 3.96-3.91 (m, 1H), 2.83 (s, 2H), 1.73 (d, 2H, J=10.8 Hz), 1.31-1.02 (m, 10H), 0.98 (s, 3H). MS (ESI): calc'd (M+H)⁺ 282.2, exp (M+H)⁺ 282.1.

### Example IIa-5

### (cis-1,5)-3,3,5-Trimethyl-5-[(2-trifluoromethyl-phenylamino)-methyl]-cyclohexanol

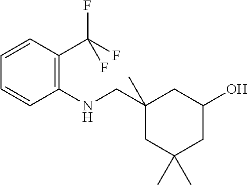

The title compound was prepared in analogy to example IIa-1 in Synthetic route 1 by using 2-trifluoromethyl-bromobenzene instead of bromobenzene. ¹H NMR (d₄-MeOD, 400 MHz), 7.41-7.36 (m, 2H), 6.88 (d, 1H, J=8.0 Hz), 6.71 (d, 1H, J=8.0 Hz), 3.97-3.94 (m, 1H), 3.00 (s, 2H), 1.78-1.75 (m, 2H), 1.29-1.03 (m, 10H), 0.98 (s, 3H). MS (ESI): calc'd (M+H)⁺ 316.2, exp (M+H)⁺ 316.2.

### Example IIa-6

### (cis-1,5)-3,3,5-Trimethyl-5-[(3-trifluoromethyl-phenylamino)-methyl]-cyclohexanol

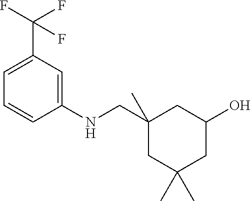

The title compound was prepared in analogy to example IIa-1 in Synthetic route 1 by using 3-trifluoromethyl-bromobenzene instead of bromobenzene. ¹H NMR (d₄-MeOD, 400 MHz), 7.22 (t, 1H, J=8.0 Hz), 6.86 (s, 1H), 6.85 (d, 1H, J=7.6 Hz), 6.78 (d, 1H, J=7.6 Hz), 3.96-3.92 (m, 1H), 2.89 (s, 2H), 1.74 (m, 2H), 1.33-1.03 (m, 10H), 0.98 (s, 3H). MS (ESI): calc'd (M+H)⁺ 316.2, exp (M+H)⁺ 316.1.

### Example IIa-7

### (cis-1,5)-3,3,5-Trimethyl-5-(p-tolylamino-methyl)-cyclohexanol

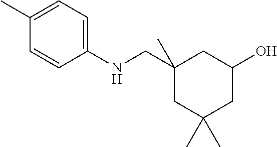

The title compound was prepared in analogy to example IIa-1 in Synthetic route 1 by using 4-methyl-bromobenzene instead of bromobenzene. ¹H NMR (d₄-MeOD, 400 MHz), 6.90 (d, 2H, J=8.0 Hz), 6.57 (d, 2H, J=8.0 Hz), 3.96-3.93 (m, 1H), 2.83 (s, 2H), 2.19 (s, 3H), 1.75-1.72 (m, 2H), 1.28-1.02 (m, 10H), 0.98 (s, 3H). MS (ESI): calc'd (M+H)⁺ 262.2, exp (M+H)⁺ 262.2.

### Example IIa-8

### (cis-1,3)-3-[(3-Methoxy-phenylamino)-methyl]-3,5,5-trimethyl-cyclohexanol

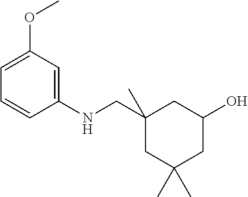

The title compound was prepared in analogy to example IIa-1 in Synthetic route 1 by using 4-methoxy-bromobenzene instead of bromobenzene. ¹H NMR (d₄-MeOD, 400 MHz), 6.97 (t, 1H, J=8.0 Hz), 6.26-6.22 (m, 2H), 6.15 (J, 1H, J=8.0 Hz), 3.96-3.90 (m, 1H), 3.73 (s, 3H), 2.84 (s, 2H), 1.75-1.72 (m, 2H), 1.28-1.03 (m, 10H), 0.98 (s, 3H). MS (ESI): calc'd (M+H)⁺ 278.2, exp (M+H)⁺ 278.2.

### Example IIa-9

### 3-[((cis-1,5)-5-Hydroxy-1,3,3-trimethyl-cyclohexylmethyl)-amino]-benzonitrile

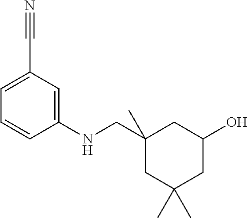

The title compound was prepared in analogy to example IIa-1 in Synthetic route 1 by using 3-nitrile-bromobenzene instead of bromobenzene. ¹H NMR (d₄-MeOD, 400 MHz), 7.21 (t, 1H, J=8.0 Hz), 6.94-6.90 (m, 2H), 6.84 (d, 1H, J=7.6 Hz), 3.96-3.90 (m, 1H), 2.88 (s, 2H), 1.76-1.72 (m, 2H), 1.30-0.99 (m, 13H). MS (ESI): calc'd (M+H)⁺ 273.2, exp (M+H)⁺ 273.2.

### Example IIa-10

### 4-[((cis-1,5)-5-Hydroxy-1,3,3-trimethyl-cyclohexylmethyl)-amino]-benzonitrile

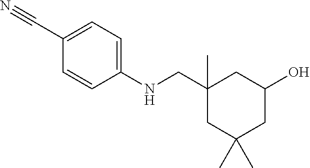

The title compound was prepared in analogy to example IIa-1 in Synthetic route 1 by using 4-nitrile-bromobenzene instead of bromobenzene. ¹H NMR (d₄-MeOD, 400 MHz), 7.35 (d, 1H, J=7.2 Hz), 6.69 (d, 1H, J=7.2 Hz), 3.94-3.85 (m, 1H), 2.91 (s, 2H), 1.74-1.68 (m, 2H), 1.26 (d, 1H, J=13.6 Hz), 1.18 (d, 1H, J=13.6 Hz), 1.10-0.99 (m, 8H), 0.95 (s, 3H). MS (ESI): calc'd (M+H)⁺ 273.0, exp (M+H)⁺ 273.0.

### Example IIa-11

### (cis-1,3)-3-[(3-Isopropoxy-phenylamino)-methyl]-3,5,5-trimethyl-cyclohexanol

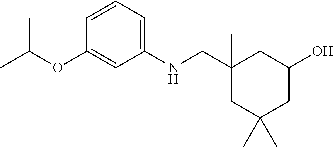

The title compound was prepared in analogy to example IIa-1 in Synthetic route 1 by using 3-isopropoxy-bromobenzene instead of bromobenzene. ¹H NMR (d₄-MeOD, 400 MHz), 6.93 (t, 1H, J=8.0 Hz), 6.22 (dt, 1H, J₁=8.4 Hz, J₂=1.6 Hz), 6.18 (t, 1H, J=1.6 Hz), 6.11 (dt, 1H, J₁=8.4 Hz, J₂=1.6 Hz), 4.52-4.48 (pent, 1H, J=6.4 Hz), 3.95-3.86 (m, 1H), 2.81 (s, 2H), 1.74-1.66 (m, 2H), 1.26 (d, 6H, J=6.0 Hz), 1.23-1.18 (m, 1H), 1.15-1.00 (m, 8H), 0.95 (s, 3H). MS (ESI): calc'd (M+H)⁺ 306, exp (M+H)⁺ 306.2.

### Example IIa-12

### (cis-1,3)-3-[(3-Isopropyl-phenylamino)-methyl]-3,5,5-trimethyl-cyclohexanol

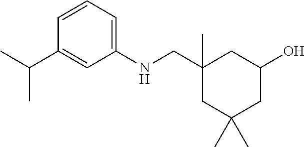

The title compound was prepared in analogy to example IIa-1 in Synthetic route 1 by using 3-isopropyl-bromobenzene instead of bromobenzene. ¹H NMR (d₄-MeOD, 400 MHz), 7.35 (t, 1H, J=8.0 Hz), 7.16-7.14 (m, 2H), 7.07 (d, 1H, J=8.0 Hz), 4.00-3.92 (m, 1H), 3.11 (s, 2H), 2.92 (m, 1H, J=7.2 Hz), 1.82-1.71 (m, 2H), 1.41 (d, 1H, J=13.6 Hz), 1.31-1.21 (m, 10H), 1.19-1.10 (m, 2H), 1.09 (s, 3H), 1.01 (s, 3H). MS (ESI): calc'd (M+H)⁺ 290.0, exp (M+H)⁺ 290.0.

### Example IIa-13

### 3-[((cis-1,5)-5-Hydroxy-1,3,3-trimethyl-cyclohexylmethyl)-amino]-benzamide

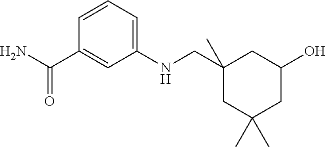

The title compound was prepared in analogy to example IIa-1 in Synthetic route 1 by using 3-formamide-bromobenzene instead of bromobenzene. ¹H NMR (d₄-MeOD, 400 MHz), 7.18-7.13 (m, 2H), 7.04 (d, 1H, J=7.6 Hz), 6.83 (d, 1H, J=7.6 Hz), 3.96-3.91 (m, 1H), 2.92 (s, 2H), 1.78-1.71 (m, 2H), 1.34-1.03 (m, 10H), 0.98 (s, 3H). MS (ESI): calc'd (M+H)⁺ 291.2, exp (M+H)⁺ 291.2.

### Example IIa-14

### (cis-1,3)-3-[(3-Methanesulfonyl-phenylamino)-methyl]-3,5,5-trimethyl-cyclohexanol

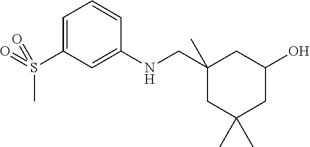

The title compound was prepared in analogy to example IIa-1 in Synthetic route 1 by using 3-methanesulfonyl-bromobenzene instead of bromobenzene. ¹H NMR (d₄-MeOD, 400 MHz), 7.31 (t, 1H, J=8.0 Hz), 7.15 (s, 1H), 7.06 (d, 1H, J=7.6 Hz), 6.94 (d, 1H, J=8.0 Hz), 3.96-3.90 (m, 1H), 3.08 (s, 3H), 2.93 (s, 2H), 1.78-1.72 (m, 2H), 1.34-1.03 (m, 10H), 0.98 (s, 3H). MS (ESI): calc'd (M+H)⁺ 326.2, exp (M+H)⁺ 326.2.

### Example IIa-15

### 3-[((cis-1,5)-5-Hydroxy-1,3,3-trimethyl-cyclohexylmethyl)-amino]-benzenesulfonamide

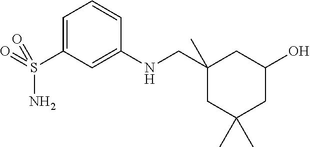

The title compound was prepared in analogy to example IIa-1 in Synthetic route 1 by using 3-sulfonamide-bromobenzene instead of bromobenzene. ¹H NMR (d₄-MeOD, 400 MHz), 7.22 (t, 1H, J=8.0 Hz), 7.15 (t, 1H, J=2.0 Hz), 7.06 (dt, 1H, J₁=8.0 Hz, J₂=0.8 Hz), 6.85 (dt, 1H, J₁=8.0 Hz, J₂=1.2 Hz), 3.95-3.87 (m, 1H), 2.88 (s, 2H), 1.74-1.68 (m, 2H), 1.27 (d, 1H, J=13.6 Hz), 1.18 (d, 1H, J=13.6 Hz), 1.08 (s, 3H), 1.06-0.95 (m, 5H), 0.94 (s, 3H). MS (ESI): calc'd (M+H)⁺ 327.2, exp (M+H)⁺ 327.2.

### Example IIa-16

### (cis-1,3)-3-[(3-Chloro-5-fluoro-phenylamino)-methyl]-3,5,5-trimethyl-cyclohexanol

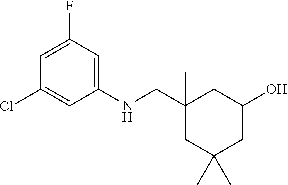

The title compound was prepared in analogy to example IIa-1 in Synthetic route 1 by using 3-chloro-5-fluoro-bromobenzene instead of bromobenzene. ¹H NMR (d₄-MeOD, 400 MHz), 6.46 (s, 1H), 6.31-6.24 (m, 2H), 3.965-3.92 (m, 1H), 2.83 (s, 2H), 1.73 (d, 2H, J=12.0 Hz), 1.34-1.03 (m, 10H), 0.98 (s, 3H). MS (ESI): calc'd (M+H)⁺ 300.2, exp (M+H)⁺ 300.2.

### Example IIa-17

### (cis-1,3)-3-[(3-Fluoro-5-trifluoromethyl-phenylamino)-methyl]-3,5,5-trimethyl-cyclohexanol

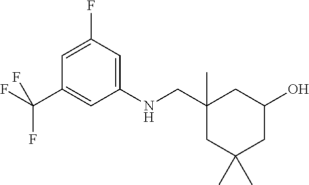

The title compound was prepared in analogy to example IIa-1 in Synthetic route 1 by using 3-fluoro-5-trifluoromethyl-bromobenzene instead of bromobenzene. ¹H NMR (CDCl₃, 400 MHz), 6.62-6.59 (m, 2H), 6.45 (d, 1H, J=11.2 Hz), 4.09-3.95 (m, 1H), 2.90 (s, 2H), 1.82-1.79 (m, 2H), 1.31 (d, 1H, J=14 Hz), 1.18-1.00 (m, 12H). MS (ESI): calc'd (M+H)⁺ 334, exp (M+H)⁺ 334.

### Example IIa-18

### (cis-1,3)-3-[(3-Chloro-5-methyl-phenylamino)-methyl]-3,5,5-trimethyl-cyclohexanol

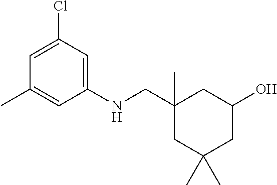

The title compound was prepared in analogy to example IIa-1 in Synthetic route 1 by using 3-chloro-5-methyl-bromobenzene instead of bromobenzene. ¹H NMR (d₄-MeOD, 400 MHz), 6.55 (d, 1H, J=1.8 Hz), 6.49 (m, 2H), 3.91 (m, 1H), 2.85 (s, 2H), 2.21 (s, 3H), 1.74-1.69 (m, 2H), 1.26 (d, 1H, J=13.2 Hz), 1.20-1.05 (m, 9H), 0.96 (s, 3H). MS (ESI): calc'd (M+H)⁺ 296.1, exp (M+H)⁺ 296.1.

### Example IIa-19

### (cis-1,3)-3-[(3,5-Dichloro-phenylamino)-methyl]-3,5,5-trimethyl-cyclohexanol

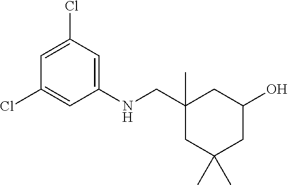

The title compound was prepared in analogy to example IIa-1 in Synthetic route 1 by using 3,5-dichloro-bromobenzene instead of bromobenzene. ¹H NMR (d₄-MeOD, 400 MHz), 6.54 (d, 1H, J=1.6 Hz), 6.48 (t, 1H, J=1.6 Hz), 3.82-3.98 (m, 1H), 2.79 (s, 2H), 1.65-1.72 (m, 2H), 1.25 (d, 1H, J=13.6 Hz), 1.18 (d, 1H, J=13.6 Hz), 1.12-0.98 (m, 8H), 0.95 (s, 3H). MS (ESI): calc'd (M+H)⁺ 316, exp (M+H)⁺ 315.9.

### Example IIa-20

### (cis-1,3)-3-[(3-Chloro-5-trifluoromethyl-phenylamino)-methyl]-3,5,5-trimethyl-cyclohexanol

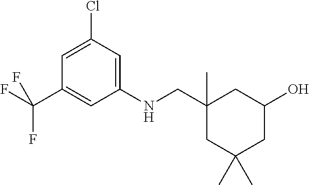

The title compound was prepared in analogy to example IIa-1 in Synthetic route 1 by using 3-chloro-5-trifluoromethyl-bromobenzene instead of bromobenzene. ¹H NMR (CDCl₃, 400 MHz), 6.81 (s, 1H), 6.66 (s, 1H), 6.63 (s, 1H), 3.98 (m, 1H), 2.84 (s, 2H), 1.77-1.74 (m, 2H), 1.26-1.22 (d, 1H, J=13.6 Hz), 1.09-0.98 (m, 9H), 0.93 (s, 3H). MS (ESI): calc'd (M+H)⁺ 350.1, exp (M+H)⁺ 350.1.

### Example IIa-21

### (cis-1,3)-3-[(3-Bromo-5-trifluoromethyl-phenylamino)-methyl]-3,5,5-trimethyl-cyclohexanol

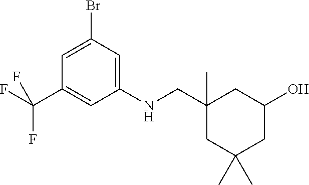

The title compound was prepared in analogy to example IIa-1 in Synthetic route 1 by using 1,3-dibromo-5-trifluoromethyl-benzene instead of bromobenzene. ¹H NMR (d₄-MeOD, 400 MHz), 7.00 (s, 1H), 6.87 (s, 1H), 6.85 (s, 1H), 3.96-3.91 (m, 1H), 2.88 (s, 2H), 1.77-1.72 (m, 2H), 1.33-1.03 (m, 10H), 0.98 (s, 3H). MS (ESI): calc'd (M+H)⁺ 394.1, exp (M+H)⁺ 394.0.

### Example IIa-22

### 3-Fluoro-5-[((cis-1,5)-5-hydroxy-1,3,3-trimethyl-cyclohexylmethyl)-amino]-benzonitrile

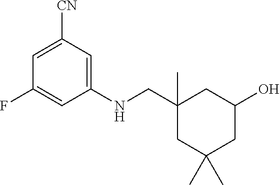

The title compound was prepared in analogy to example IIa-1 in Synthetic route 1 by using 3-bromo-5-fluoro-benzonitrile instead of bromobenzene. ¹H NMR (d₄-MeOD, 400 MHz), 6.73 (m, 1H, J=1.2 Hz), 6.62 (dt, 1H, J₁=12 Hz, J₂=1.2 Hz), 6.53 (dt, 1H, J₁=8 Hz, J₂=1.2 Hz), 3.94-3.85 (m, 1H), 2.84 (s, 2H), 1.74-1.67 (m, 2H), 1.26 (d, 1H, J=13.6 Hz), 1.15 (d, 1H, J=13.6 Hz), 1.10-0.99 (m, 8H), 0.94 (s, 3H). MS (ESI): calc'd (M+H)⁺ 291, exp (M+H)⁺ 290.8.

### Example IIa-23

### 3-Chloro-5-[((cis-1,5)-5-hydroxy-1,3,3-trimethyl-cyclohexylmethyl)-amino]-benzonitrile

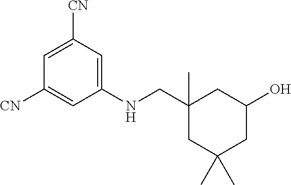

The title compound was prepared in analogy to example IIa-1 in Synthetic route 1 by using 3-bromo-5-chloro-benzonitrile instead of bromobenzene. ¹H NMR (d₄-MeOD, 400 MHz), 6.89 (t, 1H, J=2.0 Hz), 6.84 (t, 1H, J=2.0 Hz), 6.80 (t, 1H, J=1.6 Hz), 3.94-3.87 (m, 1H), 2.85 (s, 2H), 1.75-1.68 (m, 2H), 1.28 (d, 1H, J=13.6 Hz), 1.17 (d, 1H, J=13.6 Hz), 1.12-1.01 (m, 8H), 0.96 (s, 3H). MS (ESI): calc'd (M+H)⁺ 307, exp (M+H)⁺ 307.2.

### Example IIa-24

### 3-[((cis-1,5)-5-Hydroxy-1,3,3-trimethyl-cyclohexylmethyl)-amino]-5-trifluoromethyl-benzonitrile

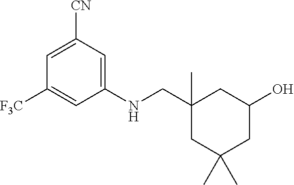

The title compound was prepared in analogy to example IIa-1 in Synthetic route 1 by using 3-bromo-5-trifluoromethyl-benzonitrile instead of bromobenzene. ¹H NMR (CDCl₃, 400 MHz), 7.07 (s, 1H), 6.94 (s, 1H), 6.91 (s, 1H), 3.94 (m, 1H), 2.85 (s, 2H), 1.77-1.74 (m, 2H), 1.26-1.22 (d, 1H, J=13.2 Hz), 1.09-0.98 (m, 9H), 0.93 (s, 3H). MS (ESI): calc'd (M+H)⁺ 341.1, exp (M+H)⁺ 341.1.

### Example IIa-25

### (cis-1,3)-3-[(3,5-Bis-trifluoromethyl-phenylamino)-methyl]-3,5,5-trimethyl-cyclohexanol

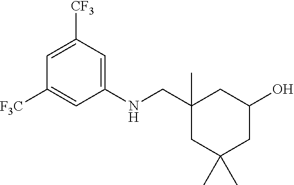

The title compound was prepared in analogy to example IIa-1 in Synthetic route 1 by using 3,5-bistrifluoromethyl-bromobenzene instead of bromobenzene. ¹H NMR (CDCl₃, 400 MHz), 7.14 (s, 1H), 6.96 (s, 2H), 4.14-4.01 (m, 1H), 2.96 (s, 2H), 1.84-1.81 (m, 2H), 1.33 (d, 1H, J=14 Hz), 1.20-1.00 (m, 12H). MS (ESI): calc'd (M+H)⁺ 384, exp (M+H)⁺ 384.

### Example IIa-26

### (cis-1,3)-3-[(2,3-Dichloro-phenylamino)-methyl]-3,5,5-trimethyl-cyclohexanol

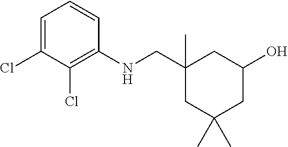

The title compound was prepared in analogy to example IIa-1 in Synthetic route 1 by using 2,3-dichloro-bromobenzene instead of bromobenzene. ¹H NMR (CDCl₃, 400 MHz), 7.07-7.03 (m, 1H), 6.78 (dd, 1H, J₁=1.2 Hz, J₂=3.6 Hz), 6.58 (dd, 1H, J₁=0.8 Hz, J₂=8.4 Hz), 4.05-3.99 (m, 1H), 2.95 (s, 2H), 1.86-1.79 (m, 2H), 1.35 (d, 1H, J=14 Hz), 1.21-1.00 (m, 12H). MS (ESI): calc'd (M+H)⁺ 316, exp (M+H)⁺ 316.

### Example IIa-27

### (cis-1,3)-3-[(3,4-Dichloro-phenylamino)-methyl]-3,5,5-trimethyl-cyclohexanol

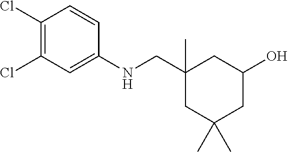

The title compound was prepared in analogy to example IIa-1 in Synthetic route 1 by using 3,4-dichloro-bromobenzene instead of bromobenzene. ¹H NMR (CDCl₃, 400 MHz), 7.18 (d, 1H, J=8.8 Hz), 6.69 (d, 1H, J=2.8 Hz), 6.47-6.44 (m, 1H), 4.03-3.97 (m, 1H), 2.85 (s, 2H), 1.82-1.77 (m, 1H), 1.29 (d, 1H, J=14 Hz), 1.17-0.99 (m, 13H). MS (ESI): calc'd (M+H)⁺ 316, exp (M+H)⁺ 316.

### Example IIa-28

### 2-Fluoro-5-[((cis-1,5)-5-hydroxy-1,3,3-trimethyl-cyclohexylmethyl)-amino]-benzonitrile

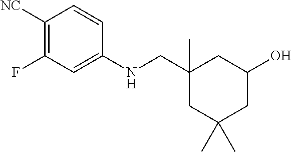

The title compound was prepared in analogy to example IIa-1 in Synthetic route 1 by using 2-fluoro-4-bromo-benzonitrile instead of bromobenzene. ¹H NMR (CDCl₃, 400 MHz), 6.97-6.91 (m, 1H), 6.78-6.72 (m, 1H), 6.69-6.67 (m, 1H), 3.98 (m, 1H), 2.85 (s, 2H), 2.77 (s, 2H), 1.77-1.74 (m, 2H), 1.26-1.22 (d, 1H, J=13.6 Hz), 1.11 (d, 1H, J=13.6 Hz), 1.09-0.94 (m, 8H), 0.89 (s, 3H). MS (ESI): calc'd (M+H)⁺ 291, exp (M+H)⁺ 290.9.

### Example IIa-29

### 2-Chloro-4-[((cis-1,5)-5-hydroxy-1,3,3-trimethyl-cyclohexylmethyl)-amino]-benzonitrile

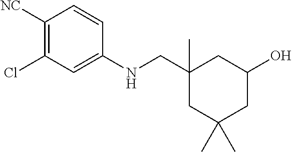

The title compound was prepared in analogy to example IIa-1 in Synthetic route 1 by using 2-chloro-4-bromo-benzonitrile instead of bromobenzene. ¹H NMR (CDCl₃, 400 MHz), 7.34 (d, 1H, J=8.8 Hz), 6.62 (d, 1H, J=2.4 Hz), 6.47 (dd, 1H, J₁=8.8 Hz, J₂=2.4 Hz), 3.98 (m, 1H), 2.90 (s, 2H), 1.78-1.75 (m, 2H), 1.26 (d, 1H, J=13.6 Hz), 1.12-0.97 (m, 9H), 0.96 (s, 3H). MS (ESI): calc'd (M+H)⁺ 307, exp (M+H)⁺ 307.2.

### Example IIa-30

### 2-Chloro-5-[((cis-1,5)-5-hydroxy-1,3,3-trimethyl-cyclohexylmethyl)-amino]-benzonitrile

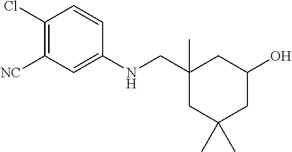

The title compound was prepared in analogy to example IIa-1 in Synthetic route 1 by using 2-chloro-5-bromo-benzonitrile instead of bromobenzene. ¹H NMR (CDCl₃, 400 MHz), 7.17 (d, 1H, J=8.8 Hz), 6.80-6.79 (d, 1H, J=2.8 Hz), 6.72 (dd, 1H, J₁=8.8 Hz, J₂=2.8 Hz), 3.98 (m, 1H), 3.17 (bs, OH), 2.82 (s, 2H), 1.77-1.74 (m, 2H), 1.25 (d, 1H, J=13.6 Hz), 1.15-0.92 (m, 9H), 0.92 (s, 3H). MS (ESI): calc'd (M+H)⁺ 307, exp (M+H)⁺ 307.2.

### Example IIa-31

### 4-[((cis-1,5)-5-Hydroxy-1,3,3-trimethyl-cyclohexylmethyl)-amino]-phthalonitrile

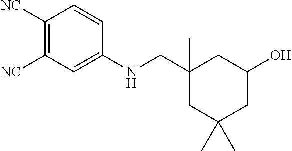

The title compound was prepared in analogy to example IIa-1 in Synthetic route 1 by using 4-bromo-phthalonitrile instead of bromobenzene. ¹H NMR (d₄-MeOD, 400 MHz), 7.52 (d, 1H, J=8.8 Hz), 7.07 (d, 1H, J=2.4 Hz), 6.95 (dd, 1H, J₁=8.8 Hz, J₂=2.4 Hz), 3.94-3.86 (m, 1H), 2.96 (s, 2H), 1.74-1.68 (m, 2H), 1.30 (d, 1H, J=13.6 Hz), 1.16 (d, 1H, J=13.6 Hz), 1.12-1.03 (m, 8H), 0.96 (s, 3H). MS (ESI): calc'd (M+H)⁺ 298, exp (M+H)⁺ 298.

### Example IIa-32

### (cis-1,3)-3-[(4-Chloro-3-trifluoromethyl-phenylamino)-methyl]-3,5,5-trimethyl-cyclohexanol

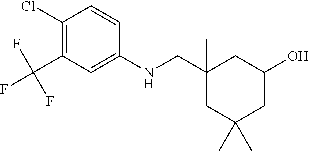

The title compound was prepared in analogy to example IIa-1 in Synthetic route 1 by using 4-bromo-1-chloro-2-trifluoromethyl-benzene instead of bromobenzene. ¹H NMR (d₄-MeOD, 400 MHz), 7.20 (d, 1H, J=8.8 Hz), 6.98 (d, 1H, J=3.2 Hz), 6.79 (dd, 1H, J₁=8.8 Hz, J₂=2.8 Hz), 3.82-3.98 (m, 1H), 2.85 (s, 2H), 1.64-1.75 (m, 2H), 1.29 (d, 1H, J=13.6 Hz), 1.17 (d, 1H, J=13.6 Hz), 1.15-0.98 (m, 8H), 0.95 (s, 3H). MS (ESI): calc'd (M+H)⁺ 350, exp (M+H)⁺ 350.0.

### Example IIa-33

### 4-[((cis-1,5)-5-Hydroxy-1,3,3-trimethyl-cyclohexylmethyl)-amino]-2-trifluoromethyl-benzonitrile

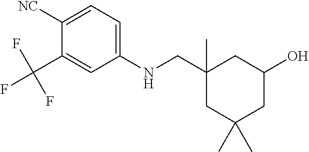

The title compound was prepared in analogy to example IIa-1 in Synthetic route 1 by using 4-bromo-2-trifluoromethyl-benzonitrile instead of bromobenzene. ¹H NMR (d₄-MeOD, 400 MHz), 7.54 (d, 1H, J=8.8 Hz), 7.03 (d, 1H, J=2.4 Hz), 6.85 (dd, 1H, J₁=8.8 Hz, J₂=2.4 Hz), 3.94-3.85 (m, 1H), 2.95 (s, 2H), 1.75-1.68 (m, 2H), 1.29 (d, 1H, J=13.6 Hz), 1.17 (d, 1H, J=13.6 Hz), 1.12-0.99 (m, 8H), 0.94 (s, 3H). MS (ESI): calc'd (M+H)⁺ 341, exp (M+H)⁺ 341.1.

### Example IIa-34

### 2-Chloro-5-[((cis-1,5)-5-hydroxy-1,3,3-trimethyl-cyclohexylmethyl)-amino]-benzenesulfonamide

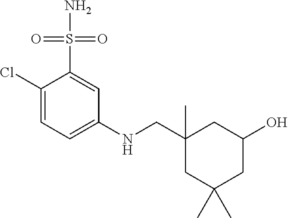

The title compound, IIa-34 was prepared according to the method shown in Synthetic route 2. The substituted benzenesulfonyl chlorides can be made by the Sandmeyer reaction of corresponding anilines. The synthesis method was generally applied to other examples such as IIa-35, IIa-36 and IIa-37, which also have the sulfonamide functional group.

### Synthetic Route 2 to Example IIa-34

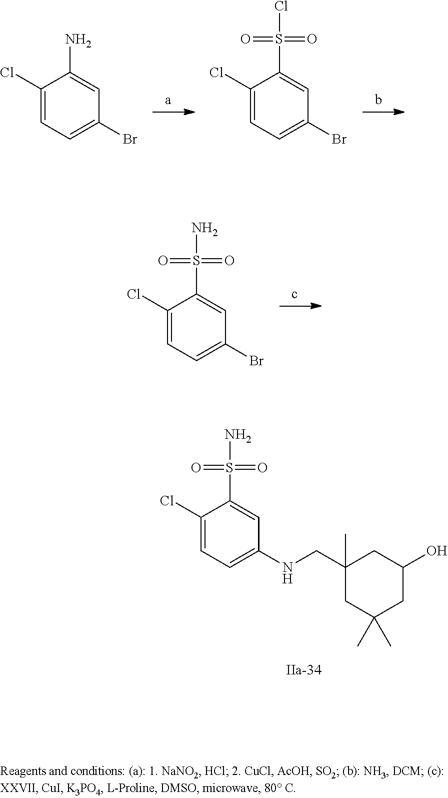

5-Bromo-2-chloro-benzenesulfonyl chloride was prepared by the following procedures. To a stirring mixture of 5-bromo-2-chloro-phenylamine (2 g, 9.6 mmol) in 10 mL of HCl, a solution of NaNO₂ (0.8 g, 11.6 mmol) in 10 mL of water was added dropwise to keep the temperature below 5° C. The diazonium salt solution was added into a SO₂ gas saturated acetic acid solution of CuCl (0.28 g, 2.88 mmol), and the mixture was stirred at room temperature for 2 h. The mixture was treated with water when the reaction was complete. The aqueous phase was extracted by ethyl acetate, and the combined organic solution was washed with water and aqueous NaHCO₃ solution, dried over Na₂SO₄, and concentrated. The crude product from Sandmeyer reaction was used in the next step reaction without further purification. The so obtained sulfonyl chloride was dissolved in 50 mL of DCM. NH₃ was bubbled into this solution at −78° C. for 10 min, and the mixture was brought to room temperature and stirred for 16 h. The reaction mixture was washed by water (50 mL), and dried under vacuum to give 1.2 g of 5-bromo-2-chloro-benzenesulfonamide (46% yield for two steps). ¹H NMR (d₆-DMSO, 400 MHz), 8.04 (d, 1H, J=2.4 Hz), 7.81 (dd, 1H, J₁=8.4 Hz, J₁=2.4 Hz), 7.65 (bs, 2H), 7.60 (d, 1H, J=8.4 Hz).

The copper catalyzed coupling reaction between amine XXVII and 5-bromo-2-chloro-benzenesulfonamide gave title compound IIa-34 as white solid. ¹H NMR (d₄-MeOD, 400 MHz), 7.34 (d, 1H, J=2.8 Hz), 7.20 (d, 1H, J=8.4 Hz), 6.77 (dd, 1H, J₁=8.4 Hz, J₁=2.8 Hz), 3.96-3.85 (m, 1H), 2.86 (s, 2H), 1.73-1.68 (m, 2H), 1.31-1.19 (m, 3H), 1.10 (s, 3H), 1.06 (s, 3H), 1.04-1.00 (m, 1H), 0.96 (s, 3H). MS (ESI): calc'd (M+H)⁺ 361.1, exp (M+H)⁺ 361.1.

### Example IIa-35

### 3-Chloro-5-[((cis-1,5)-5-hydroxy-1,3,3-trimethyl-cyclohexylmethyl)-amino]-benzenesulfonamide

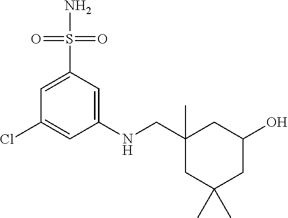

The title compound was prepared in analogy to example IIa-34 in Synthetic route 2 by using 3-bromo-5-chloro-phenylamine instead of 5-bromo-2-chloro-phenylamine. ¹H NMR (d₄-MeOD, 400 MHz), 7.03 (t, 1H, J=2.0 Hz), 6.96 (t, 1H, J=1.6 Hz), 6.77 (t, 1H, J=2.0 Hz), 3.94-3.85 (m, 1H), 2.85 (s, 2H), 1.73-1.65 (m, 2H), 1.27 (d, 1H, J=13.6 Hz), 1.18 (d, 1H, J=13.6 Hz), 1.08 (s, 3H), 1.06-0.95 (m, 5H), 0.94 (s, 3H). MS (ESI): calc'd (M+H)⁺ 361.0, exp (M+H)⁺ 361.0.

### Example IIa-36

### 3-[((cis-1,5)-5-Hydroxy-1,3,3-trimethyl-cyclohexylmethyl)-amino]-5-trifluoromethyl-benzenesulfonamide

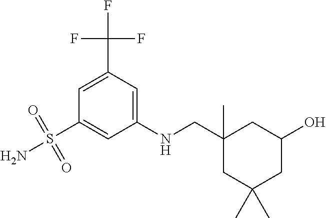

The title compound was prepared in analogy to example IIa-34 in Synthetic route 2 by using 3-bromo-5-trifluoromethyl-phenylamine instead of 5-bromo-2-chloro-phenylamine. ¹H NMR (d₄-MeOD, 400 MHz), 7.32 (s, 1H), 7.25 (s, 1H), 7.06 (s, 1H), 3.97-3.89 (m, 1H), 2.93 (s, 2H), 1.76-1.68 (m, 2H), 1.29 (d, 1H, J=13.2 Hz), 1.20 (d, 1H, J=13.2 Hz), 1.13-1.00 (m, 8H), 0.96 (s, 3H). MS (ESI): calc'd (M+H)⁺ 395, exp (M+H)⁺ 394.9.

### Example IIa-37

### 3-Fluoro-5-[((cis-1,5)-5-hydroxy-1,3,3-trimethyl-cyclohexylmethyl)-amino]-benzenesulfonamide

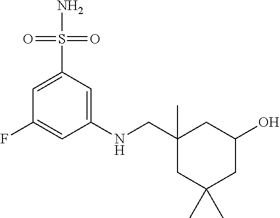

The title compound was prepared in analogy to example IIa-34 in Synthetic route 2 by using 3-bromo-5-fluoro-phenylamine instead of 5-bromo-2-chloro-phenylamine. ¹H NMR (d₄-MeOD, 400 MHz), 6.95 (t, 1H, J=1.6 Hz), 6.69 (dt, 1H, J₁=8.4 Hz, J₂=0.8 Hz), 6.51 (dt, 1H, J₁=12.0 Hz, J₂=0.8 Hz), 3.94-3.85 (m, 1H), 2.86 (s, 2H), 1.83-1.67 (m, 2H), 1.28 (d, 1H, J=13.6 Hz), 1.17 (d, 1H, J=13.6 Hz), 1.08 (s, 3H), 1.06-0.96 (m, 5H), 0.94 (s, 3H). MS (ESI): calc'd (M+H)⁺ 345.1, exp (M+H)⁺ 345.1.

### Example IIa-38

### 5-[((cis-1,5)-5-Hydroxy-1,3,3-trimethyl-cyclohexylmethyl)-amino]-2-methyl-benzenesulfonamide

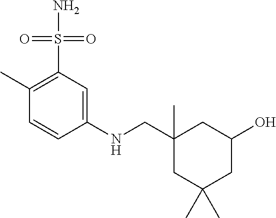

The title compound was prepared in analogy to example IIa-1 in Synthetic route 1 by using 5-bromo-2-methyl-benzenesulfonamide instead of bromobenzene. ¹H NMR (d₄-MeOD, 400 MHz), 8.11 (d, 1H, J=2.4 Hz), 7.4 (dd, 1H, J₁=8.4 Hz, J₂=2.4 Hz), 7.59 (d, 1H, J=8.4 Hz), 4.02-3.93 (m, 1H), 3.25 (d, 2H, J=5.6 Hz), 2.71 (s, 3H), 1.85-1.72 (m, 2H), 1.48 (d, 1H, J=13.6 Hz), 1.31 (s, 3H), 1.29-1.12 (m, 3H), 1.10 (s, 3H), 1.02 (s, 3H). MS (ESI): calc'd (M+H)⁺ 341.1, exp (M+H)⁺ 341.1.

### Example IIa-39

### 5-[((cis-1,5)-5-Hydroxy-1,3,3-trimethyl-cyclohexylmethyl)-amino]-2-methoxy-benzenesulfonamide

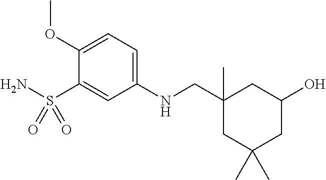

The title compound was prepared in analogy to example IIa-1 in Synthetic route 1 by using 5-bromo-2-methoxy-benzenesulfonamide instead of bromobenzene. ¹H NMR (d₄-MeOD, 400 MHz), 7.17 (s, 1H), 6.99 (d, 1H, J=8.8 Hz), 6.87 (d, 1H, J=8.8 Hz), 3.94-3.89 (m, 4H), 2.84 (s, 2H), 1.75-7.71 (m, 2H), 1.29-0.98 (m, 13H). MS (ESI): calc'd (M+H)⁺ 357.2, exp (M+H)⁺ 357.2.

### Example IIa-40

### 4-[((cis-1,5)-5-Hydroxy-1,3,3-trimethyl-cyclohexylmethyl)-amino]-2-trifluoromethoxy-benzenesulfonamide

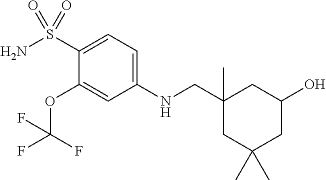

The title compound was prepared in analogy to example IIa-1 in Synthetic route 1 by using 4-bromo-2-trifluoromethoxy-benzenesulfonamide instead of bromobenzene. ¹H NMR (d₄-MeOD, 400 MHz), 7.67 (d, 1H, J=9.2 Hz), 6.69 (s, 1H), 6.60 (d, 1H, J=8.8 Hz), 3.95-3.93 (m, 1H), 2.93 (s, 2H), 1.75-1.72 (m, 2H), 1.31 (d, 1H, J=13.6 Hz), 1.21 (d, 1H, J=14.0 Hz), 1.15-1.03 (m, 8H), 0.98 (s, 3H). MS (ESI): calc'd (M+H)⁺ 411.2, exp (M+H)⁺ 411.1.

### Example IIb-1

### (cis-1,3)-3-{[(3-Chloro-phenyl)-methyl-amino]-methyl}-3,5,5-trimethyl-cyclohexanol

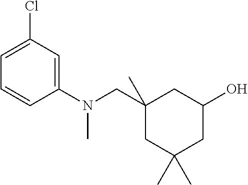

The title compound was prepared by the N-methylation of example IIa-3 according to the method shown in Scheme 1. ¹H NMR (d₄-MeOD, 400 MHz), 7.10 (t, 1H, J=8.0 Hz), 6.74-6.69 (m, 2H), 6.59 (d, 1H, J=8.0 Hz), 3.90-3.88 (m, 1H), 3.18-3.06 (m, 2H), 3.00 (s, 3H), 1.80-1.71 (m, 2H), 1.31-1.18 (m, 4H), 1.08-0.96 (m, 9H). MS (ESI): calc'd (M+H)⁺ 296.2, exp (M+H)⁺ 296.2.

### Example IIc-1

### (3-Fluoro-5-trifluoromethyl-phenyl)-(1,3,3-trimethyl-cyclohexylmethyl)-amine

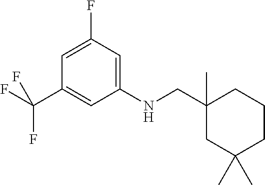

Compound of interest IIc was prepared according to Synthetic route 3.

### Synthetic Route 3 to Example IIc-1

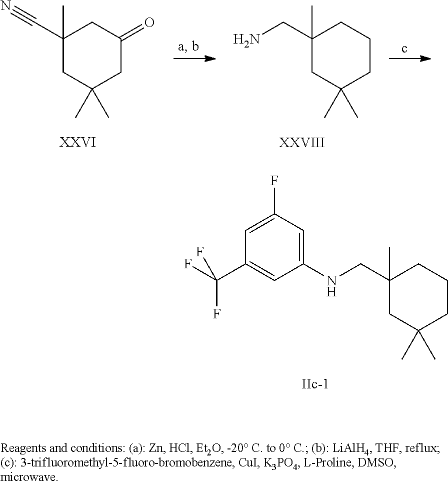

3,3-Dimethyl-cyclohexyl)-methylamine XXVIII was prepared by the following procedure. Intermediate XXVI (2.5 g, 15.1 mmol) was dissolved into 60 mL of diethyl ether that was charged and saturated with hydrogen chloride. To this solution at −20° C., 7.2 g activated zinc (110.0 mmol) was added in several portions. After stirred at 0° C. for 2 hours, the reaction mixture was poured slowly onto crushed ice. The aqueous solution was extracted with ethyl ether; and the organic phase was combined, washed with saturated NaCl, dried over Na₂SO₄ and concentrated. The residue was dissolved in 50 mL of anhydrous THF, to this solution was added 1.1 g of LiAlH₄ (30 mmol), and the reaction mixture was brought to refluxing and stirred overnight. After cooled to 0° C. (degrees Celsius), the mixture was quenched by adding of 5 mL of water and 10 mL of NaOH (2N) at 0° C. dropwisely. The mixture was filtered and the filtrate was acidified by addition of aqueous HCl (2N). After washed by ethyl acetate, the aqueous phase was neutralized to pH>9 by addition of aqueous NaOH. The aqueous phase was extracted by dichloromethylene. The combined organic layer was dried over Na₂SO₄, filtered and concentrated to give amine XXVIII. The amine was used in the copper catalyzed coupling reaction in analogy to example IIa-1 in Synthetic route 1 by using 3-trifluoromethyl-5-fluoro-bromobenzene instead of bromobenzene. The cross coupling reaction gave IIc-1 as wax. ¹H NMR (d₄-MeOD, 400 MHz), 6.72 (s, 1H), 6.55 (d, 1H, J=12.0 Hz), 6.46 (d, 1H, J=8.8 Hz), 2.95-2.86 (m, 2H), 1.61-1.57 (m, 2H), 1.37-1.32 (m, 4H), 1.26-1.22 (m, 2H), 1.03 (s, 3H), 1.01 (s, 3H), 0.94 (s, 3H). MS (ESI): calc'd (M+H)⁺ 318.2, exp (M+H)⁺ 318.1.

### Example IId-1

### Acetic acid (cis-1,5)-3,3,5-trimethyl-5-phenylaminomethyl-cyclohexyl ester

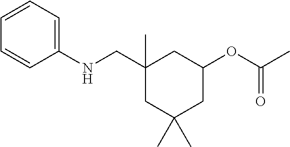

Compound of interest IId can be prepared by acylation of IIa with acyl chloride and triethylamine. ¹H NMR (d₄-MeOD, 400 MHz), 7.24 (t, 2H, J=8.0 Hz), 6.92-6.86 (m, 3H), 5.16-5.10 (m, 1H), 3.00 (s, 2H), 2.02 (s, 3H), 1.81-1.76 (m, 2H), 1.38-1.23 (m, 4H), 1.20 (s, 3H), 1.14 (s, 3H), 1.02 (s, 3H). MS (ESI): calc'd (M+H)⁺ 290.2, exp (M+H)⁺ 290.1.

### Example IIe-1

### (3,3-Difluoro-1,5,5-trimethyl-cyclohexylmethyl)-(3-fluoro-5-trifluoromethyl-phenyl)-amine

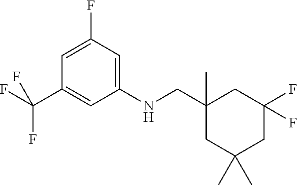

Compound of interest IIe-1 was prepared according to Synthetic route 4.

Synthetic route 4 to example IIe-1

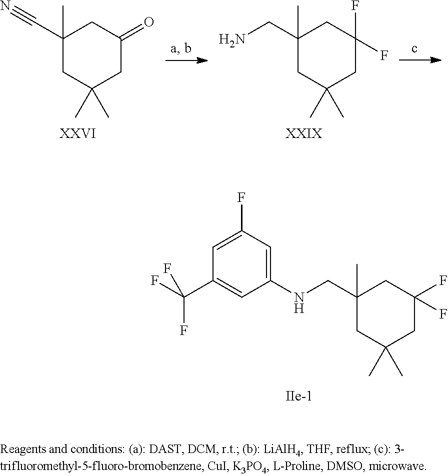

The intermediate 3,3-difluoro-5,5-dimethyl-cyclohexyl)-methylamine XXIX was prepared by following procedures. To a stirred solution of XXVI (1.0 g, 6.0 mmol) in 3 mL of dry DCM under nitrogen was added a solution of DAST (2.4 g, 14.4 mmol) in 2 mL of dry DCM, and the mixture was stirred for 2 h at r.t. The reaction mixture was washed with a.q. NaHCO₃ until CO₂ evolution ceased. The organic phase was dried over Na₂SO₄ and concentrated to give 1.1 g of crude product as yellow oil. It was used in the reduction by LiAlH₄ without further purification. Under similar reaction conditions to example IIa-1, the copper catalyzed cross-coupling of XXIX and 3-trifluoromethyl-5-fluoro-bromobenzene gave target compound IIe-1. ¹H NMR (d₄-MeOD, 400 MHz), 6.75 (s, 1H), 6.59 (d, 1H, J=12.0 Hz), 6.50 (d, 1H, J=8.8 Hz), 3.01 (s, 2H), 1.87-1.63 (m, 4H), 1.53 (d, 1H, J=14.4 Hz), 1.39 (d, 1H, J=14.4 Hz), 1.18 (s, 3H), 1.14 (s, 3H), 1.06 (s, 3H). MS (ESI): calc'd (M+H)⁺ 354.2, exp (M+H)⁺ 354.1.

### Example IIe-2

### (3-Bromo-5-trifluoromethyl-phenyl)-(3,3-difluoro-1,5,5-trimethyl-cyclohexylmethyl)-amine

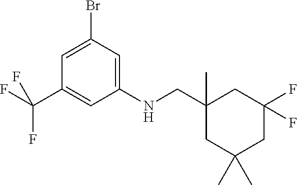

The title compound was prepared in analogy to example IIe-1 in Synthetic route 4 by using 1,3-dibromo-5-trifluoromethyl-benzene instead of 3-trifluoromethyl-5-fluoro-bromobenzene. ¹H NMR (d₄-MeOD, 400 MHz), 7.02 (s, 1H), 6.90 (s, 1H), 6.87 (s, 1H), 3.01 (s, 2H), 1.87-1.63 (m, 4H), 1.53 (d, 1H, J=14.4 Hz), 1.39 (d, 1H, J=14.4 Hz), 1.18 (s, 3H), 1.14 (s, 3H), 1.06 (s, 3H). MS (ESI): calc'd (M+H)⁺ 414.1, exp (M+H)⁺ 413.9.

### Example IIf-1

### (3-Fluoro-5-trifluoromethyl-phenyl)-((cis-1,5)-5-methoxy-1,3,3-trimethyl-cyclohexylmethyl)-amine

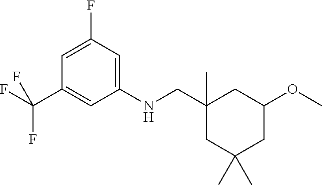

Compound of interest IIf-1 can be prepared by the synthesis route in Synthetic route 5.

### Synthetic Route 5 to Example IIf-1

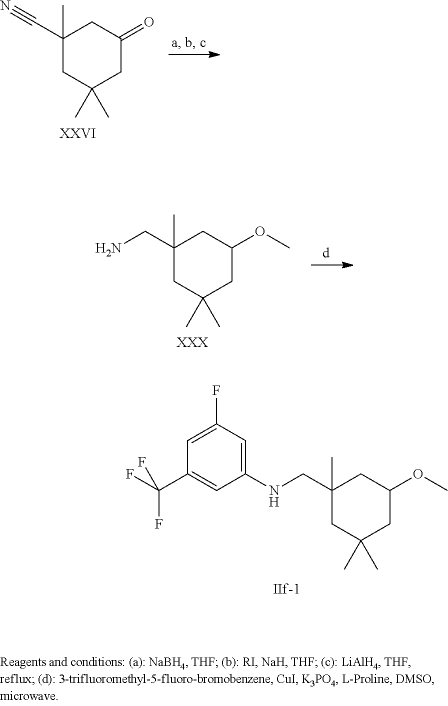

The Intermediate XXX was prepared in three steps from XXVI, by reduction of the ketone functional group, alkylation of secondary alcohol, and reduction of cyano group by LiAlH₄. To a solution of XXVI (1.65 g, 10.0 mmol) in 15 mL of EtOH was added 760 mg of NaBH₄ (20.0 mmol). The reaction mixture was stirred overnight at r.t. 5 mL of water was added to quench excess of NaBH₄. After the mixture was concentrated in vacuo, the residue was dissolved in 10 mL of water and extracted with ether. The combined organic layer was evaporated to give the secondary alcohol which was used in the alkylation reaction without further purification.

837 mg of the residue was dissolved in 5 mL of dry THF and added into a suspension solution of NaH (300 mg, 7.5 mmol) in 10 mL of dry THF at 0° C. After the mixture was stirred at r.t. for 0.5 h, 0.6 mL of iodomethane was added into the flask. The reaction mixture was stirred at r.t. for 3 h, before 20 mL of water was added. The aqueous layer was extracted with DCM twice. The combined organic layer was dried and concentrated to gave 0.8 g of methyl ether as white solid. As in the preparation of XXVIII in Synthetic route 3, the reduction of the methylation product by LiAlH₄ gave amine XXX. Compound IIf-1 was obtained by the copper catalyzed cross-coupling of XXX and 3-trifluoromethyl-5-fluoro-bromobenzene. ¹H NMR (d₄-MeOD, 400 MHz), 6.73 (s, 1H), 6.56 (d, 1H, J=12.0 Hz), 6.48 (d, 1H, J=8.8 Hz), 3.60-3.56 (m, 1H), 3.38 (s, 3H), 2.90 (s, 2H), 1.90-1.82 (m, 2H), 1.35-1.23 (m, 2H), 1.13 (s, 3H), 1.09 (s, 3H), 1.07-0.98 (m, 5H). MS (ESI): calc'd (M+H)⁺ 348.2, exp (M+H)⁺ 348.1.

### Example IIg-1

### (cis-1,3)-3-[(3-Fluoro-5-trifluoromethyl-phenylamino)-methyl]-1,3,5,5-tetramethyl-cyclohexanol

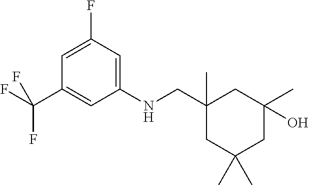

The tertiary alcohol IIg-1 was prepared according to the method in Synthetic route 6.

### Synthetic Route 6 to Example IIg-1

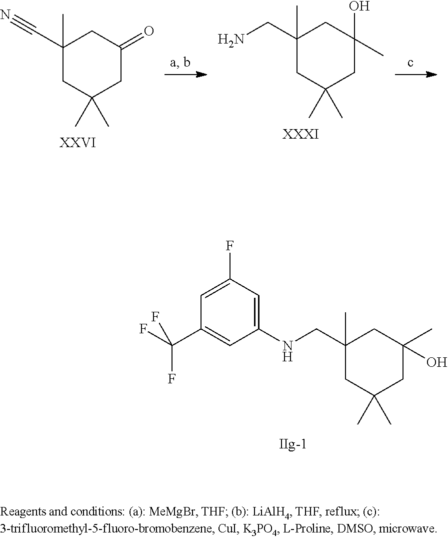

The Intermediate XXXI was prepared in two steps from XXVI by addition reaction of Grignard reagent to the ketone functional group, and reduction of the cyano functional group by LiAlH₄. A solution of MeMgBr (3M in Et₂O) was added into a solution of XXVI (1.65 g, 10.0 mmol) in 20 mL of dry THF at −40° C. The reaction mixture was stirred for 2 h at −40° C. After 4 mL of water was added into the mixture, the aqueous phase was extracted with EtOAc twice. The combined organic layer was dried over Na₂SO₄ and concentrated to give 1.7 g of tertiary alcohol as solid. The crude product was reduced by LiAlH₄ to give amine XXXI. Under the same copper catalyzed reaction conditions as in the preparation of IIa-1, the title compound IIg-1 was obtained by the coupling reaction between XXXI and 3-trifluoromethyl-5-fluoro-bromobenzene. ¹H NMR (d₄-MeOD, 400 MHz), 6.75 (s, 1H), 6.56 (d, 1H, J=12.0 Hz), 6.45 (d, 1H, J=8.8 Hz), 3.64 (d, 1H, J=12.8 Hz), 3.15 (d, 1H, J=12.4 Hz), 1.79-1.93 (m, 2H), 1.60 (d, 1H, J=14.0 Hz), 1.26-1.16 (m, 8H), 1.04 (d, 1H, J=14.8 Hz), 0.99 (s, 3H), 0.94 (s, 3H). MS (ESI): calc'd (M+H)⁺ 348.2, exp (M+H)⁺ 348.1.

### Example IIh-1

### ((cis-1,5)-5-Dimethylamino-1,3,3-trimethyl-cyclohexylmethyl)-(3-fluoro-5-trifluoromethyl-phenyl)-amine

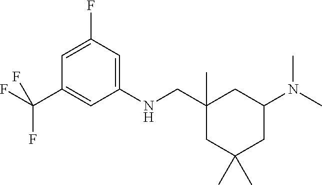

Compound of interest IIh-1 was prepared by the method in Synthetic route 7.

### Synthetic Route 7 to Example IIh-1

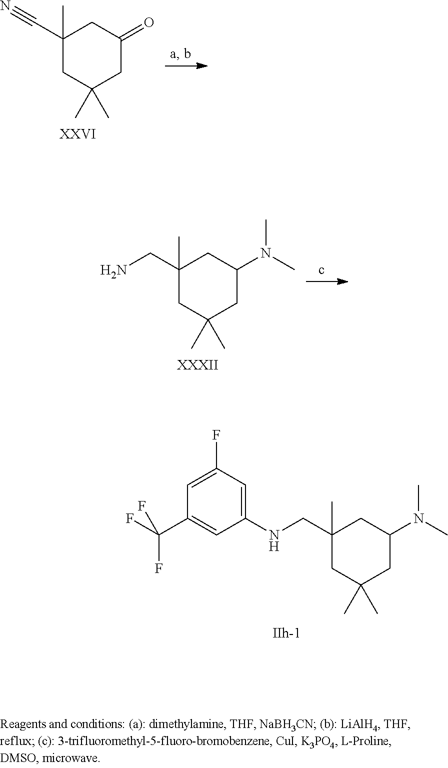

The amine Intermediate XXXII was prepared by following procedures. A solution of 1.65 g of XXX (10.0 mmol) and 0.9 g of dimethylamine (20.0 mmol) in 10 mL of dry THF was stirred for 30 min at 0° C. Then 0.8 g of NaBH₃CN (12.0 mmol) was added into the solution in several portions. After stirred overnight at r.t., the reaction mixture was poured into water and extracted with DCM. The combined organic layer was extracted with HCl (1N, 20 mL×3). And the aqueous phase was neutralized to pH>9 with NaOH (2N) and extracted with DCM (20 mL×3). The organic phase was washed with brine, dried over Na₂SO₄ and evaporated to give 1.5 g of crude product, which was reduced to XXXII by LiAlH₄. In analogy to example IIa-1 in Synthetic route 1, the title compound IIh-1 was obtained by the copper catalyzed cross-coupling of XXXII and 3-trifluoromethyl-5-fluoro-bromobenzene. ¹H NMR (d₄-MeOD, 400 MHz), 6.74 (s, 1H), 6.60 (d, 1H, J=12.0 Hz), 6.50 (d, 1H, J=8.8 Hz), 3.18-3.14 (m, 1H), 2.94 (s, 2H), 2.59 (s, 6H), 1.77-1.72 (m, 2H), 1.40-1.19 (m, 4H), 1.16 (s, 3H), 1.13 (s, 3H), 1.04 (s, 3H). MS (ESI): calc'd (M+H)⁺ 361.2, exp (M+H)⁺ 361.1.

### Example IIi-1/2

### Isomer 1: (cis-1,5)-5-[(3-Fluoro-5-trifluoromethyl-phenylamino)-methyl]-3,3-dimethyl-cyclohexanol

### Isomer 2: (trans-1,5)-5-[(3-fluoro-5-trifluoromethyl-phenylamino)-methyl]-3,3-dimethyl-cyclohexanol

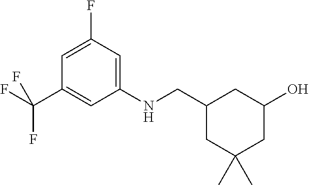

Compounds of interest IIi-1/2 (R¹=H) was prepared by the method shown in Synthetic route 8.

### Synthetic Route 8 to Example IIi-1 and IIi-2

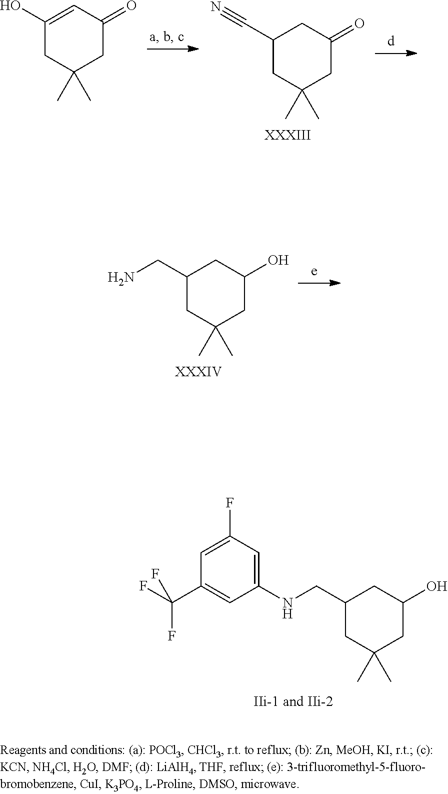

3-Hydroxy-5,5-dimethyl-cyclohex-2-enone was treated with POCl₃ to give 3-chloro-5,5-dimethyl-cyclohex-2-enone, which was reduced by zinc to afford 5,5-dimethyl-cyclohex-2-enone. The addition reaction of cyanide to 5,5-dimethyl-cyclohex-2-enone gave the cyano intermediate XXXIII, which was reduced by LiAlH₄ to give 5-aminomethyl-3,3-dimethyl-cyclohexanol XXXIV. Under the same copper catalyzed reaction conditions as the example IIa-1 in Synthetic route 1, the cross-coupling of XXXIV and 3-trifluoromethyl-5-fluoro-bromobenzene gave the cis/trans isomers IIi-1 and IIi-2, which were separated and purified by HPLC. Isomer 1: ¹H NMR (d₄-MeOD, 400 MHz), 6.66 (s, 1H), 6.51-6.48 (m, 2H), 3.79-3.73 (m, 1H), 2.99 (d, 2H, J=6.8 Hz), 2.12-2.09 (m, 1H), 1.91-1.87 (m, 1H), 1.72-1.68 (m, 1H), 1.52-1.49 (m, 1H), 1.10-1.02 (m, 1H), 1.00 (s, 3H), 0.95 (s, 3H), 0.91-0.81 (m, 2H). MS (ESI): calc'd (M+H)⁺ 319.2, exp (M+H)⁺ 318.9.

Isomer 2: ¹H NMR (d₄-MeOD, 400 MHz), 6.67 (s, 1H), 6.52-6.47 (m, 2H), 4.16-4.14 (m, 1H), 2.99-2.94 (m, 2H), 2.24-2.21 (m, 1H), 1.92-1.88 (m, 1H), 1.65-1.58 (m, 2H), 1.36-1.32 (m, 1H), 1.22-1.19 (m, 1H), 1.18 (s, 3H), 1.06-0.99 (m, 1H), 0.96 (s, 3H). MS (ESI): calc'd (M+H)⁺ 319.2, exp (M+H)⁺ 318.9.

### Example IIIa-1

### (cis-1,5)-3,3,5-Trimethyl-5-(pyridin-2-ylaminomethyl)-cyclohexanol

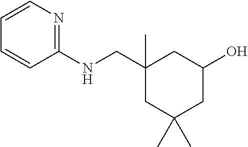

The synthesis of example IIIa-1 was carried out by the substitution of 2-chloropyridine by XXVII as shown in Synthetic route 9. The method provided a general access to other heterocyclic aromatic analogs of IIIa to IIIf.

### Synthetic Route 9 to Example IIIa-1

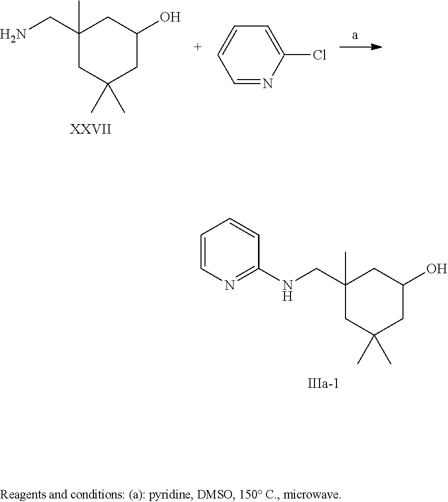

To a solution of 2-chloropyridine (138 mg, 1.22 mmol) in 2 mL of DMSO was added amine XXVII (209 mg, 1.22 mmol) and pyridine (290 mg, 3.66 mmol). The reaction was heated in the microwave at 150° C. for 0.5 h. The reaction mixture was purified by preparative HPLC to give example IIIa-1. ¹H NMR (d-MeOD, 400 MHz), 7.84 (s, 1H), 7.70 (t, 1H, J=7.6 Hz), 6.94 (d, 1H, J=8.8 Hz), 6.74 (t, 1H, J=7.2 Hz), 3.99-3.93 (m, 1H), 3.16 (s, 2H), 1.79-1.73 (m, 2H), 1.36 (d, 1H, J=14.4 Hz), 1.24-1.00 (m, 12H). MS (ESI): calc'd (M+H)⁺ 249.2, exp (M+H)⁺ 249.2.

### Example IIIa-2

### (cis-1,3)-3-[(5-Bromo-pyridin-2-ylamino)-methyl]-3,5,5-trimethyl-cyclohexanol

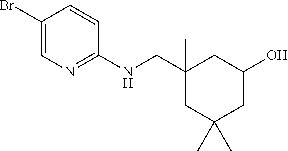

The title compound was prepared in analogy to example IIIa-1 in Synthetic route 9 by using 5-bromo-2-fluoro-pyridine instead of 2-chloropyridine. ¹H NMR (d₄-MeOD, 400 MHz), 7.97 (dd, 1H, J₁=2.0 Hz, J₂=0.8 Hz), 7.90 (dd, 1H, J₁=9.2 Hz, J₂=2.0 Hz), 7.04 (dd, 1H, J₁=9.2 Hz, J₂=0.8 Hz), 3.97-3.90 (m, 1H), 3.14 (AB, 2H, J=12.4 Hz), 1.75-1.69 (m, 2H), 1.34 (d, 1H, J=13.6 Hz), 1.18 (d, 1H, J=13.6 Hz), 1.15-1.10 (m, 4H), 1.08-1.05 (m, 4H), 0.99 (s, 3H). MS (ESI): calc'd (M+H)⁺ 327, exp (M+H)⁺ 327.1.

### Example IIIa-3

### (cis-1,5)-3,3,5-Trimethyl-5-[(6-trifluoromethyl-pyridin-2-ylamino)-methyl]-cyclohexanol

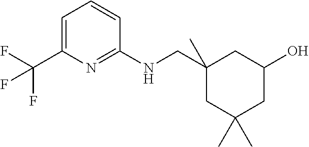

The title compound was prepared in analogy to example IIIa-1 in Synthetic route 9 by using 2-fluoro-6-trifluoromethyl-pyridine instead of 2-chloropyridine. ¹H NMR (d₄-MeOD, 400 MHz), 7.48 (t, 1H, J=8.0 Hz), 6.80 (d, 1H, J=7.2 Hz), 6.69 (d, 1H, J=8.4 Hz), 3.91 (m, 1H), 3.24-3.15 (AB, 2H, J=13.2 Hz), 1.73-1.69 (m, 2H), 1.29-1.17 (m, 2H), 1.12-0.95 (m, 8H), 0.91 (s, 3H). MS (ESI): calc'd (M+H)⁺ 317.2, exp (M+H)⁺ 317.2.

### Example IIIa-4

### (cis-1,3)-3-[(4-Chloro-pyridin-2-ylamino)-methyl]-3,5,5-trimethyl-cyclohexanol

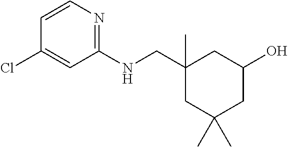

The title compound was prepared in analogy to example IIIa-1 in Synthetic route 9 by using 2-fluoro-4-chloropyridine instead of 2-chloropyridine. ¹H NMR (d₄-MeOD, 400 MHz), 7.88 (dd, J₁=8.0 Hz, J₂=1.6 Hz), 7.83 (dd, J₁=6.0 Hz, J₂=1.6 Hz), 6.78 (dd, J₁=8.0 Hz, J₂=6.0 Hz), 3.92 (m, 1H), 3.44-3.29 (m, 2H), 1.75-1.66 (m, 2H), 1.30-1.18 (m, 2H), 1.15-1.07 (m, 5H), 1.05 (s, 3H), 0.99 (s, 3H). MS (ESI): calc'd (M+H)⁺ 283.1, exp (M+H)⁺ 283.1.

### Example IIIa-5

### (cis-1,5)-3,3,5-Trimethyl-5-[(3-trifluoromethyl-pyridin-2-ylamino)-methyl]-cyclohexanol

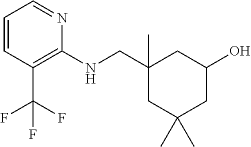

The title compound was prepared in analogy to example IIIa-1 in Synthetic route 9 by using 2-fluoro-3-trifluoromethyl-pyridine instead of 2-chloropyridine. ¹H NMR (d₄-MeOD, 400 MHz), 8.17-8.16 (d, 1H, J=4.8 Hz), 7.78-7.77 (d, 1H, J=7.6 Hz), 6.69-6.65 (q, 1H, J=5.2 Hz), 3.93 (m, 1H), 3.33-3.27 (m, 2H), 1.74-1.72 (m, 2H), 1.36-1.33 (d, 1H, J=13.6 Hz), 1.22-1.04 (m, 9H), 0.94 (s, 3H). MS (ESI): calc'd (M+H)⁺ 316, exp (M+H)⁺ 316.

### Example IIIa-6

### (cis-1,5)-3,3,5-Trimethyl-5-[(5-trifluoromethyl-pyridin-2-ylamino)-methyl]-cyclohexanol

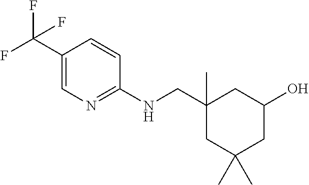

The title compound was prepared in analogy to example IIIa-1 in Synthetic route 9 by using 2-fluoro-5-trifluoromethyl-pyridine instead of 2-chloropyridine. ¹H NMR (d₄-MeOD, 400 MHz), 8.20 (s, 1H), 7.90 (d, 1H, J=9.6 Hz), 7.10 (d, 1H, J=9.6 Hz), 3.99-3.90 (m, 1H), 3.24 (AB, 2H, J=13.6 Hz), 1.77-1.69 (m, 2H), 1.35 (d, 1H, J=13.6 Hz), 1.20 (d, 1H, J=13.6 Hz), 1.15-1.12 (m, 4H), 1.14-1.05 (m, 4H), 0.99 (s, 3H). MS (ESI): calc'd (M+H)⁺ 317.2, exp (M+H)⁺ 317.2.

### Example IIIa-7

### 6-[((cis-1,5)-5-Hydroxy-1,3,3-trimethyl-cyclohexylmethyl)-amino]-pyridine-2-sulfonic acid amide

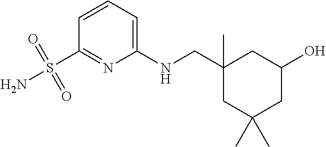

The title compound was prepared by the method shown in Synthetic route 10.

### Synthetic Route 10 to Example IIIa-7

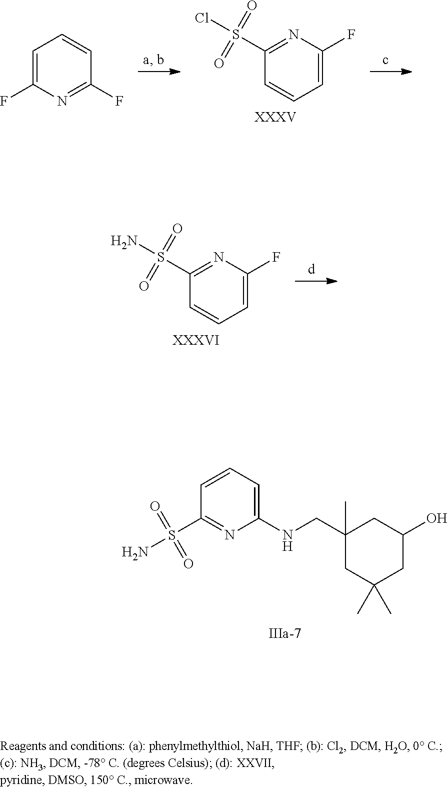

2-Benzylthio-6-fluoropyridine was prepared by starting from 2,6-difluoropyridine. To a cold solution of NaH (1.06 g, 43.96 mmol) in 170 mL of THF, was added dropwise of phenylmethylthiol (3 g, 24.15 mmol) in 15 mL of THF. After the mixture was stirred at 0° C. for 0.5 h, a solution of 2,6-difluoropyridine (2.8 g, 24.15 mmol) in 15 mL of THF was added into the flask. The reaction mixture was stirred at r.t for 3 h before 100 mL of water was added. The aqueous phase was extracted by DCM, and the combined organic phase was washed with brine, dried over Na₂SO₄, and concentrated. The residue was purified by silica gel chromatography to give 3.5 g of 2-benzylthio-6-fluoropyridine.

6-Fluoropyridine-2-sulfonamide XXXVI was prepared by the oxidation of 2-benzylthio-6-fluoropyridine to sulfonyl chloride and treatment with ammonia. A solution of 2-benzylthio-6-fluoropyridine (3 g, 13.7 mmol) in 75 mL of DCM and 60 mL of H₂O was cooled under an ice-water bath, to this cold solution was bubbled chlorine gas for a total of 1.5 hours. Then aqueous sodium metabisulphite solution was added to the mixture. After the mixture was extracted by DCM, the combined organic phase was washed with water, dried over Na₂SO₄, and concentrated to give sulfonyl chloride XXXV. The crude XXXV was used for the ammonia treatment without further purification. To a solution of crude sulfonyl chloride XXXV (2.7 g) in 250 mL of DCM at −78° C., was bubbled NH₃ for 10 minutes. Then the reaction was stirred at r.t overnight. The mixture was filtered and concentrated. The residue was purified by chromatography to give 1.0 g of sulfonamide XXXVI. ¹H NMR (d₆-DMSO, 400 MHz), 8.26 (dd, 1H, J₁=15.6 Hz, J₂=8.0 Hz), 7.88 (m, 1H), 7.62 (bs, 2H), 7.48 (dd, 1H, J₁=8.4 Hz, J₂=2.4 Hz).

The title compound IIIa-7 was prepared in analogy to example IIIa-1 in Synthetic route 9 by using 6-fluoropyridine-2-sulfonamide XXXVI instead of 2-chloropyridine. ¹H NMR (d₄-MeOD, 400 MHz), 7.54 (dd, 1H, J₁=8.8 Hz, J₂=7.6 Hz), 7.08 (d, 1H, J=7.2 Hz), 6.68 (d, 2H, J=8.8 Hz), 3.91 (m, 1H), 2.66 (m, 2H), 1.72 (m, 2H), 1.26 (d, 1H, J=13.2 Hz), 1.20 (d, 1H, J=13.2 Hz), 1.16-0.97 (m, 8H), 0.93 (s, 3H). MS (ESI): calc'd (M+H)⁺ 327.8, exp (M+H)⁺ 327.8.

### Example IIIa-8

### 2-[((cis-1,5)-5-Hydroxy-1,3,3-trimethyl-cyclohexylmethyl)-amino]-pyridine-4-sulfonic acid amide

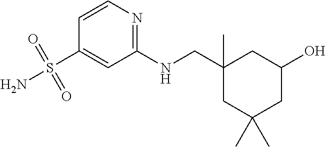

The title compound was prepared in analogy to example IIIa-1 in Synthetic route 9 by using 2-chloro-pyridine-4-sulfonamide instead of 2-chloropyridine. ¹H NMR (d₄-MeOD, 400 MHz), 7.96 (d, 1H, J=6.8 Hz), 7.56 (s, 1H), 7.13 (d, 1H, J=6.8 Hz), 3.97 (m, 1H), 3.26 (m, 2H), 1.75 (d, 2H), 1.38-1.28 (m, 2H), 1.21-1.06 (m, 8H), 0.99 (s, 3H). MS (ESI): calc'd (M+H)⁺ 328, exp (M+H)⁺ 328.2.

### Example IIIa-9

(cis-1,5)-3,3,5-Trimethyl-5-[(4-methyl-pyrimidin-2-ylamino)-methyl]-cyclohexanol

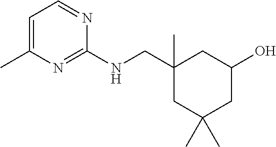

The title compound was prepared in analogy to example IIIa-1 in Synthetic route 9 by using 2-chloro-4-methyl-pyrimidine instead of 2-chloropyridine. ¹H NMR (d₄-MeOD, 400 MHz), 8.48-8.15 (m, 1H), 6.85 (d, 1H, J=6.0 Hz), 4.09-3.88 (m, 1H), 3.42-3.31 (m, 2H), 2.54 (s, 3H), 1.75-1.69 (m, 2H), 1.28 (d, 1H, J=13.6 Hz), 1.19-1.01 (m, 9H), 0.97 (s, 3H). MS (ESI): calc'd (M+H)⁺ 264.2, exp (M+H)⁺ 264.2.

### Example IIIa-10

### (cis-1,5)-3,3,5-Trimethyl-5-[(4-trifluoromethyl-pyrimidin-2-ylamino)-methyl]-cyclohexanol

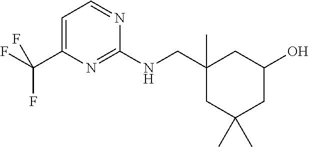

The title compound was prepared in analogy to example IIIa-1 in Synthetic route 9 by using 2-chloro-4-trifluoromethyl-pyrimidine instead of 2-chloropyridine. ¹H NMR (d₄-MeOD, 400 MHz), 8.47 (s, 1H), 6.83 (d, 1H, J=8.8 Hz), 3.93-3.85 (m, 1H), 3.27-3.21 (m, 2H), 1.73-1.68 (m, 2H), 1.20 (dd, 2H, J₁=24.4 Hz, J₂=13.6 Hz), 1.11-0.96 (m, 8H), 0.94 (s, 3H). MS (ESI): calc'd (M+H)⁺ 318.2, exp (M+H)⁺ 318.9.

### Example IIIa-11

### (cis-1,3)-3-[(4-Methoxy-pyrimidin-2-ylamino)-methyl]-3,5,5-trimethyl-cyclohexanol

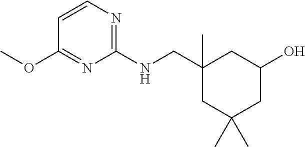

The title compound was prepared in analogy to example IIIa-1 in Synthetic route 9 by using 2-chloro-4-methoxy-pyrimidine instead of 2-chloropyridine. ¹H NMR (d₄-MeOD, 400 MHz), 8.0 (d, 1H, J=7.6 Hz), 6.38 (d, 1H, J=7.6 Hz), 4.09 (s, 3H), 3.91 (m, 1H), 3.42-3.29 (m, 2H), 1.74-1.71 (m, 2H), 1.28 (d, 1H, J=13.6 Hz), 1.18 (d, 1H, J=13.6 Hz), 1.12 (s, 3H), 1.05-0.94 (m, 8H). MS (ESI): calc'd (M+H)⁺ 280, exp (M+H)⁺ 280.2.

### Example IIIa-12

### (cis-1,3)-3-[(4,6-Dimethoxy-pyrimidin-2-ylamino)-methyl]-3,5,5-trimethyl-cyclohexanol

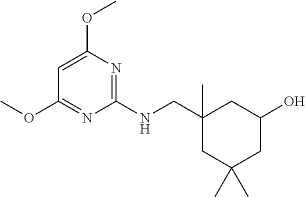

The title compound was prepared in analogy to example IIIa-1 in Synthetic route 9 by using 2-chloro-4,6-dimethoxy-pyrimidine instead of 2-chloropyridine. ¹H NMR (d₆-DMSO, 400 MHz), 7.17 (s, 1H), 5.77 (bs, 1H), 3.77 (s, 6H), 3.66 (m, 1H), 3.14-3.00 (m, 2H), 1.55-1.49 (m, 2H), 1.12-1.06 (m, 2H), 0.99-0.83 (m, 11H). MS (ESI): calc'd (M+H)⁺ 310, exp (M+H)⁺ 310.2.

### Example IIIa-13

### (cis-1,3)-3-[(4,6-Dimethyl-pyrimidin-2-ylamino)-methyl]-3,5,5-trimethyl-cyclohexanol

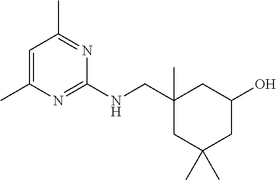

The title compound was prepared in analogy to example IIIa-1 in Synthetic route 9 by using 2-chloro-4,6-dimethyl-pyrimidine instead of 2-chloropyridine. ¹H NMR (d₄-MeOD, 400 MHz), 6.81 (m, 1H), 4.04-3.95 (m, 1H), 3.51-3.40 (m, 2H), 2.53 (s, 6H), 1.95-1.75 (m, 2H), 1.50-1.20 (m, 4H), 1.18-1.09 (m, 6H), 1.03 (s, 1H). MS (ESI): calc'd (M+H)⁺ 278, exp (M+H)⁺ 278.1.

### Example IIIa-14

### (cis-1,3)-3-[(4-Chloro-5-methoxy-pyrimidin-2-ylamino)-methyl]-3,5,5-trimethyl-cyclohexanol

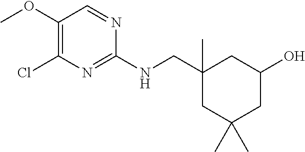

The title compound was prepared in analogy to example IIIa-1 in Synthetic route 9 by using 2,4-dichloro-5-methoxy-pyrimidine instead of 2-chloropyridine. ¹H NMR (d₄-MeOD, 400 MHz), 7.62 (s, 1H), 3.93 (s, 3H), 3.41-3.35 (m, 1H), 3.49-3.36 (m, 1H), 3.26-3.13 (m, 1H), 1.72-1.65 (m, 2H), 1.25-1.17 (m, 2H), 1.07 (s, 3H), 1.04-0.98 (m, 5H), 0.95 (s, 3H). MS (ESI): calc'd (M+H)⁺ 314.2, exp (M+H)⁺ 314.2.

### Example IIIb-1

### (cis-1,3)-3-[(6-Chloro-pyrazin-2-ylamino)-methyl]-3,5,5-trimethyl-cyclohexanol

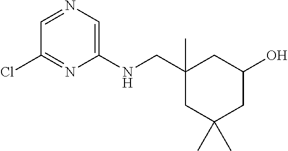

The title compound IIIb-1 was prepared in analogy to example IIIa-1 in Synthetic route 9 by using 2-chloro-6-fluoro-pyrazine instead of 2-chloropyridine. ¹H NMR (d₄-MeOD, 400 MHz), 7.78 (s, 1H), 7.54 (s, 1H), 3.88 (m, 1H), 3.15 (m, 2H), 1.71-1.67 (m, 2H), 1.22 (d, 1H, J=13.2 Hz), 1.16 (d, 1H, J=13.2 Hz), 1.06-0.96 (m, 8H), 0.93 (s, 3H). MS (ESI): calc'd (M+H)⁺ 284.2, exp (M+H)⁺ 284.2.

### Example IIIb-2

### (cis-1,5)-3,3,5-Trimethyl-5-[(6-methyl-pyrazin-2-ylamino)-methyl]-cyclohexanol

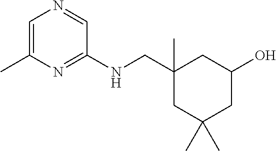

The title compound IIIb-2 was prepared in analogy to example IIIa-1 in Synthetic route 9 by using 2-fluoro-6-methyl-pyrazine instead of 2-chloropyridine. ¹H NMR (d₄-MeOD, 400 MHz), 7.94 (s, 1H), 7.61 (s, 1H), 3.98-3.90 (m, 1H), 3.27-3.24 (m, 2H), 2.42 (s, 1H), 1.72-1.68 (m, 2H), 1.30 (d, 1H, J=13.2 Hz), 1.22 (d, 1H, J=13.2 Hz), 1.12-1.02 (m, 8H), 0.98 (s, 3H). MS (ESI): calc'd (M+H)⁺ 264.2, exp (M+H)⁺ 264.2.

### Example IIIb-3

### (cis-1,3)-3-[(6-Methoxy-pyrazin-2-ylamino)-methyl]-3,5,5-trimethyl-cyclohexanol

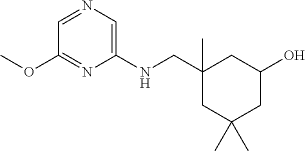

The title compound IIIb-3 was prepared in analogy to example IIIa-1 in Synthetic route 9 by using 2-fluoro-6-methoxy-pyrazine instead of 2-chloropyridine. ¹H NMR (d₄-MeOD, 400 MHz), 7.49 (s, 1H), 7.30 (s, 1H), 4.00 (s, 3H), 3.91 (m, 1H), 3.25 (m, 2H), 1.76-1.68 (m, 2H), 1.26-0.99 (m, 10H), 0.94 (s, 3H). MS (ESI): calc'd (M+H)⁺ 280.2, exp (M+H)⁺ 280.2.

### Example IIIc-1

### (cis-1,3)-3-(Benzoxazol-2-ylaminomethyl)-3,5,5-trimethyl-cyclohexanol

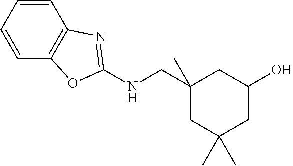

The title compound IIIc-1 was prepared as shown in Synthetic route 11.

### Synthetic Route 11 to Example IIIc-1

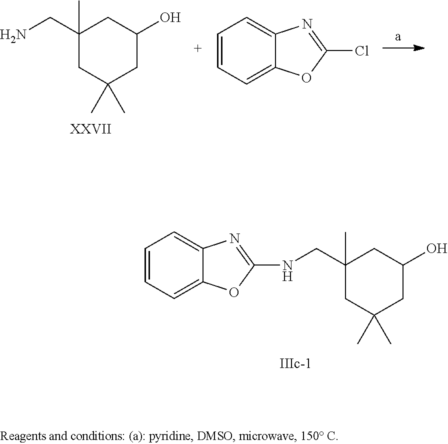

To a solution of 2-chloro-benzoxazole (230 mg, 1.5 mmol) in 2 mL of DMSO was added 3-aminomethyl-3,5,5-trimethyl-cyclohexanol XXVII (257 mg, 1.5 mmol) and pyridine (290 mg, 3.7 mmol). The reaction was heated in the microwave at 150° C. for 0.5 h. The reaction mixture was purified by preparative HPLC to give example IIIc-1 as powder. ¹H NMR (d₄-MeOD, 400 MHz), 7.26 (t, 1H, J=8.0 Hz), 7.15 (t, 1H, J=7.6 Hz), 7.05 (t, 1H, J=7.6 Hz), 3.96-3.91 (m, 1H), 3.33 (s, 2H), 1.77-1.73 (m, 2H), 1.29-1.17 (m, 2H), 1.14-0.98 (m, 11H). MS (ESI): calc'd (M+H)⁺ 289.2, exp (M+H)⁺ 289.1.

### Example IIIc-2

### Benzoxazol-2-yl-(3,3-difluoro-1,5,5-trimethyl-cyclohexylmethyl)-amine

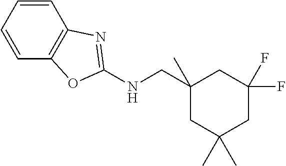

The title compound IIIc-2 was prepared in analogy to example IIIc-1 in Synthetic route 11 by using (3,3-difluoro-1,5,5-trimethyl-cyclohexyl)-methylamine 5-1 instead of amine XXVII. ¹H NMR (d₄-MeOD, 400 MHz), 7.29-7.26 (m, 2H), 7.16 (t, 1H, J=7.6 Hz), 7.04 (t, 1H, J=7.6 Hz), 3.34 (m, 2H), 1.87-1.54 (m, 2H), 1.52-1.33 (m, 3H), 1.19-1.07 (m, 10H). MS (ESI): calc'd (M+H)⁺ 309.2, exp (M+H)⁺ 309.2.

### Example IIIc-3

### Benzoxazol-2-yl-((cis-1,5)-5-methoxy-1,3,3-trimethyl-cyclohexylmethyl)-amine

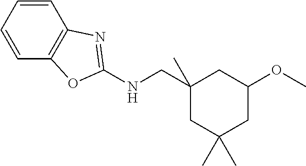

The title compound IIIc-3 was prepared in analogy to example IIIc-1 in Synthetic route 11 by using (5-methoxy-1,3,3-trimethyl-cyclohexyl)-methylamine XXX instead of amine XXVII. ¹H NMR (d₄-MeOD, 400 MHz), 7.26 (m, 2H), 7.15 (t, 1H, J=8.0 Hz), 7.03 (t, 1H, J=8.0 Hz), 3.63-3.50 (m, 1H), 3.37 (s, 3H), 3.21 (s, 2H), 1.88-1.86 (m, 2H), 1.32-1.26 (m, 2H), 1.15 (s, 3H), 1.14-0.99 (m, 8H). MS (ESI): calc'd (M+H)⁺ 303.2, exp (M+H)⁺ 303.2.

### Example IIIc-4

### (cis-1,3)-3-(Benzothiazol-2-ylaminomethyl)-3,5,5-trimethyl-cyclohexanol

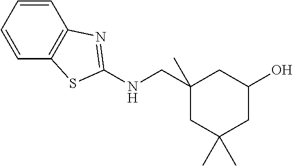

The title compound IIIc-4 was prepared in analogy to example IIIc-1 in Synthetic route 11 by using 2-chloro-benzothiazole instead of 2-chloro-benzoxazole. ¹H NMR (CDCl₃, 400 MHz), 11.70 (s, 1H), 7.62-7.57 (m, 2H), 7.50 (t, 1H, J=8.0 Hz), 7.31 (t, 1H, J=8.0 Hz), 4.06-4.02 (m, 1H), 3.24 (m, 1H), 3.10 (m, 1H), 1.86-1.78 (m, 2H), 1.38 (d, 1H, J=13.6 Hz), 1.26-1.03 (m, 13H). MS (ESI): calc'd (M+H)⁺ 305.2, exp (M+H)⁺ 305.1.

### Example IIIc-5

### (cis-1,3)-3-[(6-Fluoro-benzothiazol-2-ylamino)-methyl]-3,5,5-trimethyl-cyclohexanol

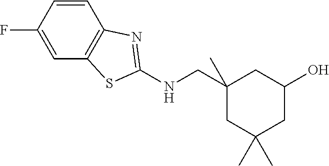

The title compound IIIc-4 was prepared in analogy to example IIIc-1 in Synthetic route 11 by using 2-chloro-6-fluoro-benzothiazole instead of 2-chloro-benzoxazole. ¹H NMR (CDCl₃, 400 MHz), 11.4 (s, 1H), 7.55-7.52 (m, 1H), 7.36-7.34 (m, 1H), 7.22-7.18 (m, 1H), 4.06-3.96 (m, 1H), 3.20 (d, 1H, J=13.2 Hz), 3.10 (d, 1H, J=13.2 Hz), 1.87-1.78 (m, 2H), 1.36-1.01 (m, 13H). MS (ESI): calc'd (M+H)⁺ 323.2, exp (M+H)⁺ 323.2

### Example IIId-1

### (cis-1,3)-3-(Benzo[d]isoxazol-3-ylaminomethyl)-3,5,5-trimethyl-cyclohexanol

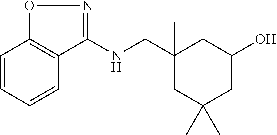

The title compound was prepared by synthetic methods described in Synthetic route 12.

### Synthetic Route 12 to Example IIId-1

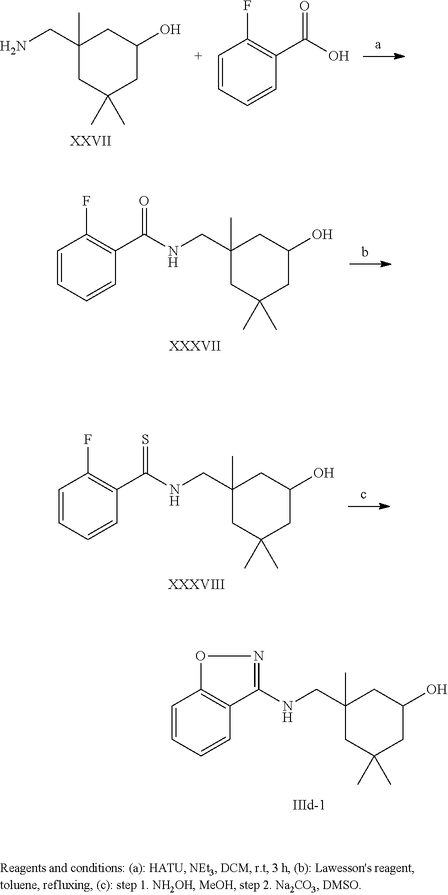

To a solution of 2-fluoro-benzoic acid (1.40 g, 10 mmol) and amine XXVII (1.71 g, 10 mmol) in 20 mL of DCM was added HATU (3.8 g, 10 mmol) and NEt₃ (1.4 mL, 10 mmol). The reaction was stirred at r.t. for 3 h and LC-MS (ESI) indicated that the reaction completed. The reaction was partitioned between water and DCM, the organic phase was dried and concentrated. The residue was purified with silica-gel column chromatography to afford amide XXXVII (2.89 g, yield: 98%).

The solution of XXXVII (1.46 g, 5 mmol) and Lawesson's reagent (2.02 g, 5 mmol) in toluene (20 mL) was stirred under refluxing overnight. The solvent was removed and the residue was purified with preparative HPLC to give thioamide XXXVIII (0.46 g, yield: 30%).

To a solution of XXXVIII (62 mg, 0.2 mmol) in MeOH (5 mL) was added hydroxylamine (0.2 mL). The reaction mixture was stirred at 80° C. for 15 hours. The solvent was removed and the residue was partitioned between water and ethyl acetate. The organic phase was dried and concentrated. The residue was dissolved in 3 mL of DMSO, and to this solution was added Na₂CO₃ (32 mg, 0.3 mmol). After stirred at 140° C. overnight, the reaction mixture was partitioned between water and ethyl acetate, and the organic phase was dried and concentrated. The residue was purified with preparative HPLC to give IIId-1 (14 mg, yield: 24%). ¹H NMR (d₄-MeOD, 400 MHz), 7.86 (d, 1H, J=7.6 Hz), 7.54 (t, 1H, J=7.6 Hz), 7.38 (d, 1H, J=8.0 Hz), 7.26 (t, 1H, J=7.6 Hz), 3.96-3.93 (m, 1H), 3.15 (s, 2H), 1.82-1.72 (m, 2H), 1.33-1.20 (m, 3H), 1.17 (s, 3H), 1.10 (s, 3H), 1.07-1.03 (m, 1H), 0.99 (s, 3H). MS (ESI): calc'd (M+H)⁺ 289.2, exp (M+H)⁺ 289.2.

### Example IIId-2

### (cis-1,3)-3-[(1H-Indazol-3-ylamino)-methyl]-3,5,5-trimethyl-cyclohexanol

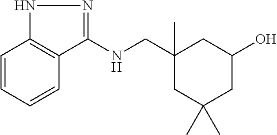

The title compound was prepared by treating the thioamide XXXVIII with hydrazine for ring formation (Synthetic route 13).

### Synthetic Route 13 to Example IIId-2

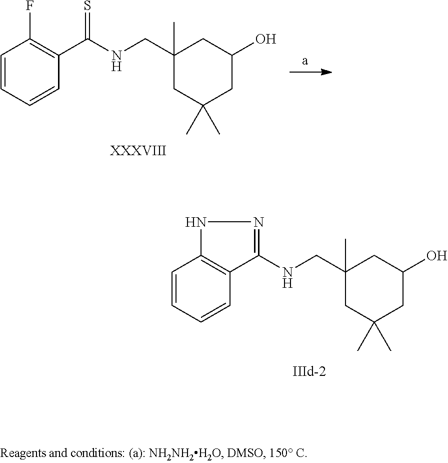

To a solution of Compound XXXVIII (40 mg, 0.13 mmol) in DMSO (2 mL) was added hydrazine (0.5 mL). The reaction mixture was stirred at 150° C. for 2 hours. LC-MS (ESI) indicated that the reaction was completed. The mixture was purified with preparative HPLC to give IIId-2 (23 mg, yield: 62%). ¹H NMR (d₄-MeOD, 400 MHz), 7.74 (d, 1H, J=8.0 Hz), 7.30-7.25 (m, 2H), 6.97 (t, 1H, J=7.6 Hz), 3.96-3.93 (m, 1H), 3.18 (s, 2H), 1.80-1.72 (m, 2H), 1.34-1.24 (m, 3H), 1.21 (s, 3H), 1.10 (s, 3H), 1.09-1.04 (m, 1H), 0.99 (s, 3H). MS (ESI): calc'd (M+H)⁺ 288.2, exp (M+H)⁺ 288.2.

### Example IIIe-1

### (cis-1,5)-3,3,5-Trimethyl-5-(quinazolin-2-ylaminomethyl)-cyclohexanol

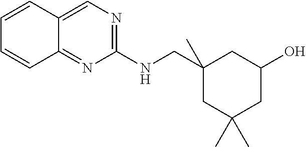

The title compound was prepared in analogy to example IIIa-1 in Synthetic route 9 by using 2-chloro-quinazoline instead of 2-chloropyridine. ¹H NMR (d₄-MeOD, 400 MHz), 9.03 (s, 1H), 7.77-7.69 (m, 2H), 7.53 (d, 1H, J=8.4 Hz), 7.26 (t, 1H, J=8.0 Hz), 3.96-3.92 (m, 1H), 3.38 (s, 2H), 1.79-1.71 (m, 2H), 1.38-1.08 (m, 10H), 0.98 (s, 3H). MS (ESI): calc'd (M+H)⁺ 300.2, exp (M+H)⁺ 300.1.

### Example IIIf-1

### (cis-1,3)-3-(Isoquinolin-1-ylaminomethyl)-3,5,5-trimethyl-cyclohexanol

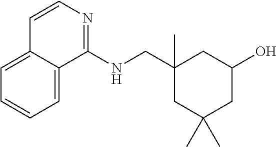

The title compound was prepared in analogy to example IIIa-1 in Synthetic route 9 by using 1-chloro-isoquinoline instead of 2-chloropyridine. ¹H NMR (CDCl₃, 400 MHz), 8.76 (m, 1H), 8.45 (d, 1H, J=8.4 Hz), 7.76 (t, 1H, J=8.0 Hz), 7.77 (t, 1H, J=8.0 Hz), 7.39 (d, 1H, J=7.2 Hz), 6.81 (d, 1H, J=6.8 Hz), 6.48 (bs, 1H), 4.12 (bs, 1H), 3.92 (m, 1H), 3.39 (m, 1H), 1.53 (m, 2H), 1.48-1.32 (m, 2H), 1.29-1.23 (m, 2H), 1.07 (s. 3H), 1.01 (s, 3H), 0.92 (s, 3H). MS (ESI): calc'd (M+H)⁺ 299.2, exp (M+H)⁺ 299.2.

### Example IVa-1

### (cis-1,5)-5-Hydroxy-1,3,3-trimethyl-cyclohexanecarboxylic acid phenylamide

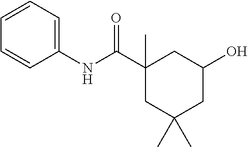

The title compound was prepared by the procedures as Synthetic route 14. The acid XXXIX was obtained by the hydrolysis of XXVI with concentrated HCl. After treatment with thionyl chloride, it was coupled with aniline to give amide XL. Compound IVa-1 was made by treating XL with NaBH₄.

### Synthetic Route 14 to Example IVa-1

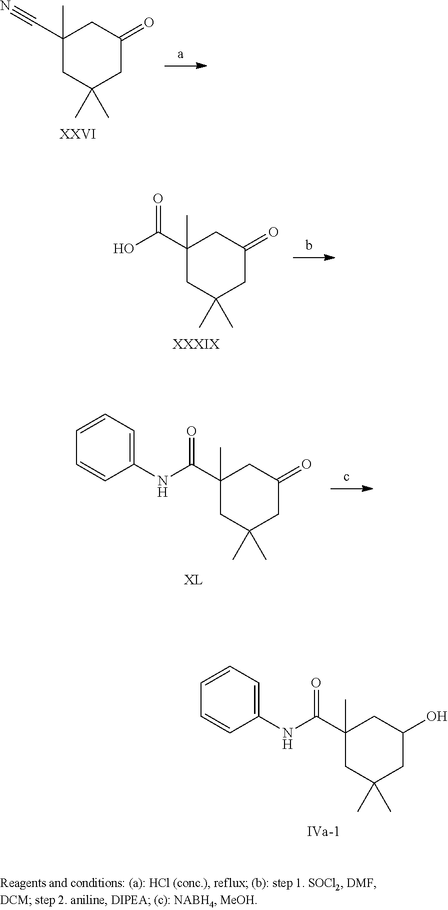

The Intermediate XXXIX, 1,3,3-trimethyl-5-oxo-cyclohexanecarboxylic acid, was made from XXVI by following procedure. The solution of 10.2 g of XXVI (61.7 mmol) in 100 mL of conc. HCl was heated to reflux overnight with stirring. When the reaction was complete, the solution was neutralized to pH=3 with NaOH (3N), and the precipitate was collected, washed with water and dried in vacuo to give 11.0 g of XXXIX as white solid (96.5% yield). ¹H NMR (CDCl₃, 400 MHz), 2.95 (d, 1H, J=14.4 Hz), 2.32 (d, 1H, J=14.4 Hz), 2.17 (s, 2H), 2.05 (d, 1H, J=14.8 Hz), 1.64 (d, 1H, J=14.4 Hz), 1.38 (s, 3H), 1.07 (s, 3H), 0.90 (s, 3H).

To a solution of the acid XXXIX (145 mg, 0.79 mmol) in 5 mL of CH₂Cl₂ was added SOCl₂ (0.5 mL), and the mixture was heated to reflux for 1 hr. After cooling, the solvent and excess SOCl₂ was evaporated under reduced pressure. The residue was dissolved in 10 mL of ClCH₂CH₂Cl. To this solution was added aniline (88 mg, 0.95 mmol) and N-methylmorpholine (0.2 mL) at r.t. After refluxed for 2 hr, the reaction mixture was cooled down and washed with 10 mL of HCl (1N) and saline. The organic layer was dried over anhydrous Na₂SO₄ and concentrated. The residue was dissolved in 10 mL of MeOH, to which was added NaBH₄ (50 mg, 1.3 mmol) at r.t. The mixture was stirred for 1 hr before evaporated and worked up with water and ethyl acetate. The organic phase was dried and concentrated, the so obtained residue was purified by column chromatography to afford IVa-1 as wax solid (250 mg). ¹H NMR (CDCl₃, 400 MHz), 8.45 (bs, 1H), 7.49 (d, 2H, J=8.4 Hz), 7.30 (t, 2H, J=7.2 Hz), 7.11 (t, 1H, J=7.2 Hz), 4.20-4.18 (m, 1H), 2.35-2.30 (m, 1H), 2.03-2.00 (m, 1H), 1.62-1.44 (m, 3H), 1.34-1.25 (m, 4H), 1.05 (s, 3H), 0.95 (s, 3H). MS (ESI): calc'd (M+H)⁺ 262, exp (M+H)⁺ 262.

### Example IVa-2

### (cis-1,5)-5-Hydroxy-1,3,3-trimethyl-cyclohexanecarboxylic acid(4-fluoro-phenyl)-amide

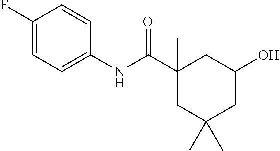

The title compound was prepared in analogy to example IVa-1 in Synthetic route 14 by using 4-fluoro-phenylamine instead of aniline. ¹H NMR (d₄-MeOD, 400 MHz), 7.48-7.52 (m, 2H), 7.03-7.07 (m, 2H), 4.06 (m, 1H), 1.89-1.85 (m, 2H), 1.74 (d, 1H, J=14 Hz), 1.655-1.612 (m, 1H), 1.55 (d, 1H, J=13.6 Hz), 1.512 (s, 3H), 1.34-1.29 (m, 1H), 1.06 (s, 3H), 1.02 (s, 3H). MS (ESI): calc'd (M+H)⁺ 280, exp (M+H)⁺ 280.

### Example IVa-3

### (cis-1,5)-5-Hydroxy-1,3,3-trimethyl-cyclohexanecarboxylic acid(3-chloro-phenyl)-amide

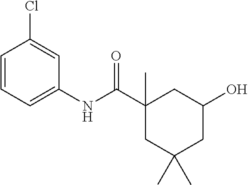

The title compound was prepared in analogy to example IVa-1 in Synthetic route 14 by using 3-chloro-phenylamine instead of aniline. ¹H NMR (CDCl₃, 400 MHz), 8.67 (bs, 1H), 7.62 (s, 1H), 7.36 (d, 1H, J=8.4 Hz), 7.21 (t, 1H, J=8.0 Hz), 7.07 (d, 1H, J=8.0 Hz), 4.25-4.23 (m, 1H), 2.35-2.30 (m, 1H), 2.03-2.00 (m, 1H), 1.62-1.44 (m, 3H), 1.34-1.25 (m, 4H), 1.04 (s, 3H), 0.97 (s, 3H). MS (ESI): calc'd (M+H)⁺ 296, exp (M+H)⁺ 296.

### Example IVa-4

### (cis-1,5)-5-Hydroxy-1,3,3-trimethyl-cyclohexanecarboxylic acid(3-trifluoromethyl-phenyl)-amide

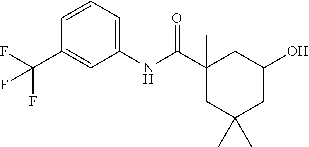

The title compound was prepared in analogy to example IVa-1 in Synthetic route 14 by using 3-trifluoromethyl-phenylamine instead of aniline. ¹H NMR (CDCl₃, 400 MHz), 8.71 (bs, 1H), 7.80 (s, 1H), 7.72 (d, 1H, J=8.0 Hz), 7.42 (t, 1H, J=8.0 Hz), 7.35 (d, 1H, J=8.0 Hz), 4.28-4.26 (m, 1H), 2.37-2.32 (m, 1H), 2.11-2.03 (m, 1H), 1.65-1.46 (m, 3H), 1.36-1.24 (m, 4H), 1.04 (s, 3H), 0.97 (s, 3H). MS (ESI): calc'd (M+H)⁺ 330, exp (M+H)⁺ 330.

### Example IVa-5

### (cis-1,5)-5-Hydroxy-1,3,3-trimethyl-cyclohexanecarboxylic acid(3-isopropyl-phenyl)-amide

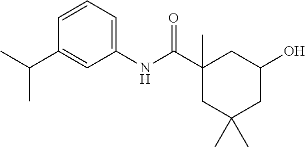

The title compound was prepared in analogy to example IVa-1 in Synthetic route 14 by using 3-isopropyl-phenylamine instead of aniline. ¹H NMR (CDCl₃, 400 MHz), 7.96 (s, 1H), 7.38-7.23 (m, 3H), 7.02-7.00 (m, 1H), 4.23-4.18 (m, 1H), 2.94-2.87 (m, 1H), 2.33 (dd, 1H, J₁=14.4 Hz, J₂=4.8 Hz), 1.94 (d, 1H, J=14.4 Hz), 1.64-1.46 (m, 7H), 1.36 (m, 6H), 1.05 (s, 3H), 0.99 (s, 3H). MS (ESI): calc'd (M+H)⁺ 304, exp (M+H)⁺ 304.

### Example IVa-6

### (cis-1,5)-5-Hydroxy-1,3,3-trimethyl-cyclohexanecarboxylic acid(3-tert-butyl-phenyl)-amide

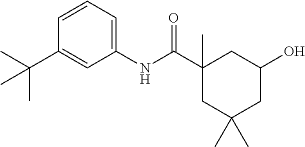

The title compound was prepared in analogy to example IVa-1 in Synthetic route 14 by using 3-tert-butyl-phenylamine instead of aniline. ¹H NMR (CDCl₃, 400 MHz), 7.85 (s, 1H), 7.49-7.48 (m, 1H), 7.48-7.11 (m, 3H), 4.23-4.19 (m, 1H), 2.35-2.30 (m, 1H), 1.93 (d, 1H, J=14 Hz) 1.65-1.32 (m, 16H), 1.04 (s, 3H), 1.00 (s, 3H). MS (ESI): calc'd (M+H)⁺ 318, exp (M+H)⁺ 318.

### Example IVa-7

### (cis-1,5)-5-Hydroxy-1,3,3-trimethyl-cyclohexanecarboxylic acid(3-ethoxy-phenyl)-amide

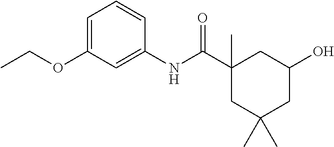

The title compound was prepared in analogy to example IVa-1 in Synthetic route 14 by using 3-ethoxy-phenylamine instead of aniline. ¹H NMR (d₄-MeOD, 400 MHz), 7.27-7.21 (m, 2H), 7.07-7.04 (m, 1H), 6.69-6.66 (m, 1H), 4.08-4.01 (m, 3H), 1.95-1.85 (m, 2H), 1.78-1.57 (m, 3H), 1.42-1.30 (m, 7H), 1.06 (s, 3H), 1.03 (s, 3H). MS (ESI): calc'd (M+H)⁺ 306, exp (M+H)⁺ 306.

### Example IVa-8

### (cis-1,5)-5-Hydroxy-1,3,3-trimethyl-cyclohexanecarboxylic acid(3-trifluoromethoxy-phenyl)-amide

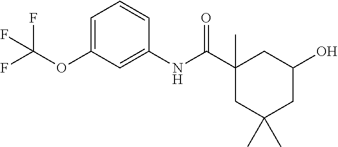

The title compound was prepared in analogy to example IVa-1 in Synthetic route 14 by using 3-trifluoromethoxy-phenylamine instead of aniline. ¹H NMR (d₄-MeOD, 400 MHz), 7.68 (s, 1H), 7.52-7.49 (m, 1H), 7.40 (t, 1H, J=8.0 Hz), 7.03-7.00 (m, 1H), 4.08-4.05 (m, 1H), 1.95-1.85 (m, 2H), 1.78-1.57 (m, 3H), 1.42 (s, 3H), 1.35-1.30 (m, 1H), 1.08 (s, 3H), 1.04 (s, 3H). MS (ESI): calc'd (M+H)⁺ 346, exp (M+H)⁺ 346.

### Example IVa-9

### (cis-1,5)-5-Hydroxy-1,3,3-trimethyl-cyclohexanecarboxylic acid(3-methanesulfonyl-phenyl)-amide

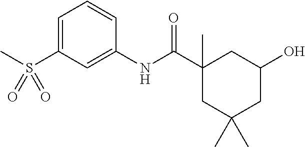

The title compound was prepared in analogy to example IVa-1 in Synthetic route 14 by using 1-bromo-3-methanesulfonyl-benzene instead of aniline. ¹H NMR (CDCl₃, 400 MHz), 9.32 (s, 1H), 8.38-8.35 (m, 2H), 8.02 (d, 1H, J=7.6 Hz), 7.90-7.86 (m, 1H), 4.64-4.61 (m, 1H), 3.46 (s, 3H), 2.69-2.64 (m, 1H), 2.39 (d, 1H, J=14 Hz), 2.05-2.00 (m, 1H), 1.90-1.70 (m, 6H), 1.39 (s, 3H), 1.37 (s, 3H). MS (ESI): calc'd (M+H)⁺ 340, exp (M+H)⁺ 340.

### Example IVa-10

### (cis-1,5)-5-Hydroxy-1,3,3-trimethyl-cyclohexanecarboxylic acid(3-sulfamoyl-phenyl)-amide

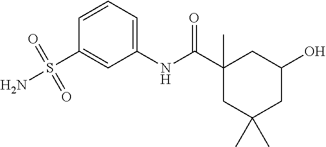

The title compound was prepared in analogy to example IVa-1 in Synthetic route 14 by using 3-bromo-benzenesulfonamide instead of aniline. ¹H NMR (d₆-DMSO, 400 MHz), 9.58 (s, 1H), 8.18 (s, 1H), 7.85-7.83 (m, 2H), 7.51-7.46 (m, 2H), 7.33 (s, 2H), 3.91-3.82 (m, 1H), 1.81 (d, 1H, J=9.6 Hz), 1.62-1.56 (m, 3H), 1.47 (d, 1H, J=13.6 Hz), 1.33 (s, 3H), 1.10-1.05 (m, 1H), 1.02 (s, 3H), 0.97 (s, 3H). MS (ESI): calc'd (M+H)⁺ 341, exp (M+H)⁺ 341.

### Example IVa-11

### (cis-1,5)-5-Hydroxy-1,3,3-trimethyl-cyclohexanecarboxylic acid(3,5-difluoro-phenyl)-amide

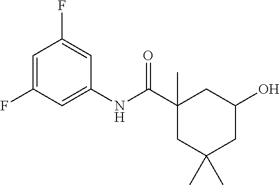

The title compound was prepared in analogy to example IVa-1 in Synthetic route 14 by using 3,5-difluoro-phenylamine instead of aniline. ¹H NMR (CDCl₃, 400 MHz), 8.84 (s, 1H), 7.15-7.12 (m, 2H), 6.56-6.52 (m, 1H), 4.29-4.27 (m, 1H), 2.32-2.31 (m, 1H), 2.06-2.02 (m, 1H) 1.63-1.28 (m, 7H), 1.02 (s, 3H), 0.95 (s, 3H). MS (ESI): calc'd (M+H)⁺ 298, exp (M+H)⁺ 298.

### Example IVa-12

### (cis-1,5)-5-Hydroxy-1,3,3-trimethyl-cyclohexanecarboxylic acid(3-chloro-5-fluoro-phenyl)amide

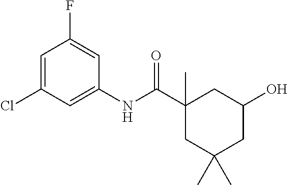

The title compound was prepared in analogy to example IVa-1 in Synthetic route 14 by using 3-chloro-5-fluoro-phenylamine instead of aniline. ¹H NMR (CDCl₃, 400 MHz), 8.80 (s, 1H), 7.37-7.27 (m, 2H), 6.84-6.81 (m, 1H), 4.30-4.28 (m, 1H), 2.38-2.31 (m, 1H), 2.05 (d, 1H, J=14.4 Hz), 1.63-1.28 (m, 7H), 1.02 (s, 3H), 0.96 (s, 3H). MS (ESI): calc'd (M+H)⁺ 314, exp (M+H)⁺ 314.

### Example IVa-13

### (cis-1,5)-5-Hydroxy-1,3,3-trimethyl-cyclohexanecarboxylic acid(3-fluoro-5-trifluoromethyl-phenyl)-amide

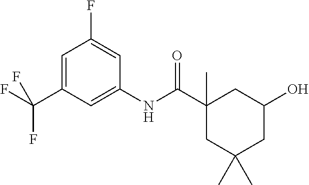

The title compound was prepared in analogy to example IVa-1 in Synthetic route 14 by using 3-fluoro-5-trifluoromethyl-phenylamine instead of aniline. ¹H NMR (CDCl₃, 400 MHz), 8.92 (s, 1H), 7.74-7.71 (m, 1H), 7.45 (s, 1H), 7.06 (d, 1H, J=8.4 Hz), 4.33-4.31 (m, 1H), 2.37-2.33 (m, 1H), 2.10-2.07 (m, 1H), 1.66-1.29 (m, 7H), 1.03 (s, 3H), 0.98 (s, 3H). MS (ESI): calc'd (M+H)⁺ 348, exp (M+H)⁺ 348.

### Example IVa-14

### (cis-1,5)-5-Hydroxy-1,3,3-trimethyl-cyclohexanecarboxylic acid(3-chloro-5-trifluoromethyl-phenyl)-amide

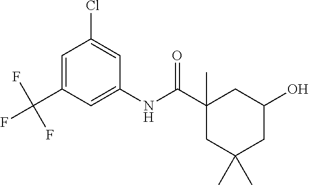

The title compound was prepared in analogy to example IVa-1 in Synthetic route 14 by using 3-chloro-5-trifluoromethyl-phenylamine instead of aniline. ¹H NMR (d₄-MeOD, 400 MHz), 7.98 (s, 1H), 7.90 (s, 1H), 7.41 (s, 1H), 4.08-4.05 (m, 1H), 1.89-1.87 (m, 2H), 1.74-1.64 (m, 3H), 1.41 (s, 3H), 1.33-1.24 (m, 1H), 1.08 (s, 3H), 1.02 (s, 3H). MS (ESI): calc'd (M+H)⁺ 364, exp (M+H)⁺ 364.

### Example IVa-15

### (cis-1,5)-5-Hydroxy-1,3,3-trimethyl-cyclohexanecarboxylic acid(3-bromo-5-trifluoromethyl-phenyl)-amide

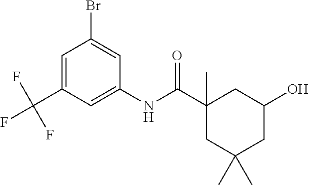

The title compound was prepared in analogy to example IVa-1 in Synthetic route 14 by using 3-bromo-5-trifluoromethyl-phenylamine instead of aniline. ¹H NMR (d₄-MeOD, 400 MHz), 8.12 (s, 1H), 7.95 (s, 1H), 7.54 (s, 1H), 4.08-4.05 (m, 1H), 1.89-1.87 (m, 2H), 1.74-1.64 (m, 3H), 1.41 (s, 3H), 1.33-1.24 (m, 1H), 1.08 (s, 3H), 1.02 (s, 3H). MS (ESI): calc'd (M+H)⁺ 408, exp (M+H)⁺ 408.

### Example IVa-16

### (cis-1,5)-5-Hydroxy-1,3,3-trimethyl-cyclohexanecarboxylic acid(3-methoxy-5-trifluoromethyl-phenyl)-amide

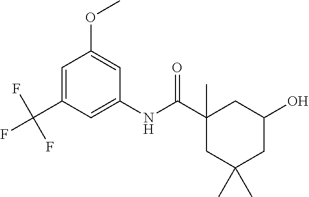

The title compound was prepared in analogy to example IVa-1 in Synthetic route 14 by using 3-methoxy-5-trifluoromethyl-phenylamine instead of aniline. ¹H NMR (d₄-MeOD, 400 MHz), 7.52 (s, 1H), 7.49 (s, 1H), 6.92 (s, 1H), 4.08-4.05 (m, 1H), 3.86 (s, 3H), 1.89-1.87 (m, 2H), 1.74-1.64 (m, 3H), 1.41 (s, 3H), 1.33-1.24 (m, 1H), 1.08 (s, 3H), 1.02 (s, 3H). MS (ESI): calc'd (M+H)⁺ 360, exp (M+H)⁺ 360.

### Example IVa-17

### (cis-1,5)-5-Hydroxy-1,3,3-trimethyl-cyclohexanecarboxylic acid(3,5-dichloro-phenyl)-amide

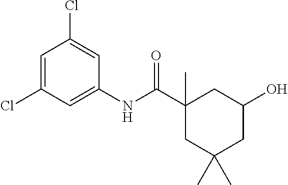

The title compound was prepared in analogy to example IVa-1 in Synthetic route 14 by using 3,5-dichloro-phenylamine instead of aniline. ¹H NMR (CDCl₃, 400 MHz), 8.69 (bs, 1H), 7.50 (d, 2H, J=2.0 Hz), 7.10 (t, 1H, J=2.0 Hz), 4.31-4.29 (m, 1H), 2.34-2.29 (m, 1H), 1.97-1.93 (m, 2H), 1.64-1.48 (m, 3H), 1.32 (s, 3H), 1.06 (s, 3H), 0.98 (s, 3H). MS (ESI): calc'd (M+H)⁺ 320, exp (M+H)⁺ 320.

### Example IVa-18

### (cis-1,5)-5-Hydroxy-1,3,3-trimethyl-cyclohexanecarboxylic acid(3,5-dimethoxy-phenyl)-amide

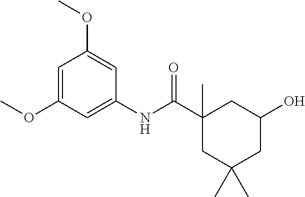

The title compound was prepared in analogy to example IVa-1 in Synthetic route 14 by using 3,5-dimethoxy-phenylamine instead of aniline. ¹H NMR (CDCl₃, 400 MHz), 8.03 (bs, 1H), 6.77 (d, 2H, J=2.4 Hz), 6.26 (t, 1H, J=2.4 Hz), 4.23-4.21 (m, 1H), 3.80 (s, 6H), 2.34-2.29 (m, 1H), 1.97-1.93 (m, 2H), 1.64-1.48 (m, 3H), 1.32 (s, 3H), 1.06 (s, 3H), 0.98 (s, 3H). MS (ESI): calc'd (M+H)⁺ 322, exp (M+H)⁺ 322.

### Example IVa-19

### (cis-1,5)-5-Hydroxy-1,3,3-trimethyl-cyclohexanecarboxylic acid(4-chloro-3-fluoro-phenyl)amide

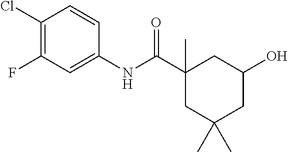

The title compound was prepared in analogy to example IVa-1 in Synthetic route 14 by using 3-fluoro-4-chloro-phenylamine instead of aniline. ¹H NMR (CDCl₃, 400 MHz), 8.41 (s, 1H), 7.69-7.67 (m, 1H), 7.35-7.31 (m, 1H), 7.11-7.06 (m, 1H), 4.28-4.26 (m, 1H), 2.36-2.31 (m, 1H), 2.02 (d, 1H, J=14 Hz), 1.64-1.31 (m, 7H), 1.04 (s, 3H), 0.98 (s, 3H). MS (ESI): calc'd (M+H)⁺ 314, exp (M+H)⁺ 314.

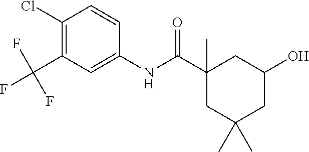

The title compound was prepared in analogy to example IVa-1 in Synthetic route 14 by using 3-trifluoromethyl-4-chloro-phenylamine instead of aniline. ¹H NMR (d₄-MeOD, 400 MHz), 8.09 (d, 1H, J=2.4 Hz), 7.82 (dd, 1H, J₁=8.8 Hz, J₂=2.4 Hz), 7.55 (d, 1H, J=8.8 Hz), 4.08-4.05 (m, 1H), 1.89-1.87 (m, 2H), 1.74-1.64 (m, 3H), 1.41 (s, 3H), 1.33-1.24 (m, 1H), 1.08 (s, 3H), 1.02 (s, 3H). MS (ESI): calc'd (M+H)⁺ 364, exp (M+H)⁺ 364.

### Example IVa-23

### (cis-1,5)-5-Hydroxy-1,3,3-trimethyl-cyclohexanecarboxylic acid(4-methoxy-3-trifluoromethyl-phenyl)-amide

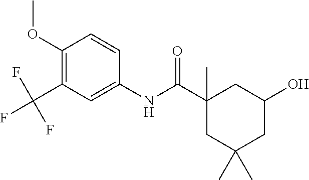

The title compound was prepared in analogy to example IVa-1 in Synthetic route 14 by using 3-trifluoromethyl-4-methoxy-phenylamine instead of aniline. ¹H NMR (d₄-MeOD, 400 MHz), 7.77 (d, 1H, J=2.4 Hz), 7.71 (dd, 1H, J₁=8.8 Hz, J₂=3.2 Hz), 7.15 (d, 1H, J=3.2 Hz), 4.08-4.05 (m, 1H), 3.90 (s, 3H), 1.89-1.87 (m, 2H), 1.74-1.64 (m, 3H), 1.41 (s, 3H), 1.33-1.24 (m, 1H), 1.08 (s, 3H), 1.02 (s, 3H). MS (ESI): calc'd (M+H)⁺ 360, exp (M+H)⁺ 360.

### Example IVa-24

### (cis-1,5)-5-Hydroxy-1,3,3-trimethyl-cyclohexanecarboxylic acid(4-cyano-3-trifluoromethyl-phenyl)-amide

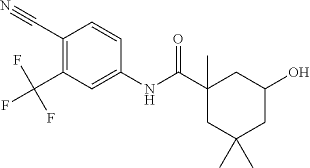

### Example IVa-20

### (cis-1,5)-5-Hydroxy-1,3,3-trimethyl-cyclohexanecarboxylic acid(3,4-dichloro-phenyl)-amide

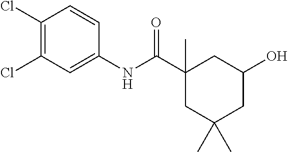

The title compound was prepared in analogy to example IVa-1 in Synthetic route 14 by using 3,4-dichloro-phenylamine instead of aniline. ¹H NMR (CDCl₃, 400 MHz), 8.56 (s, 1H), 7.74 (d, 1H, J=1.2 Hz), 7.38-7.36 (m, 2H), 4.30-4.27 (m, 1H), 2.36-2.31 (m, 1H), 2.03 (d, 1H, J=14 Hz), 1.64-1.30 (m, 7H), 1.03 (s, 3H), 0.97 (s, 3H). MS (ESI): calc'd (M+H)⁺ 330, exp (M+H)⁺ 330.

### Example IVa-21

### (cis-1,5)-5-Hydroxy-1,3,3-trimethyl-cyclohexanecarboxylic acid(4-fluoro-3-trifluoromethyl-phenyl)-amide

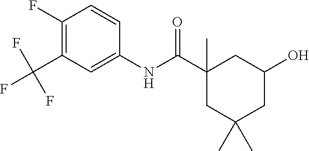

The title compound was prepared in analogy to example IVa-1 in Synthetic route 14 by using 3-trifluoromethyl-4-fluoro-phenylamine instead of aniline. ¹H NMR (d₄-MeOD, 400 MHz), 7.97-7.95 (m, 1H), 7.83-7.79 (m, 1H), 7.29 (t, 1H, J=9.6 Hz), 4.08-4.05 (m, 1H), 1.89-1.87 (m, 2H), 1.74-1.64 (m, 3H), 1.41 (s, 3H), 1.33-1.24 (m, 1H), 1.08 (s, 3H), 1.02 (s, 3H). MS (ESI): calc'd (M+H)⁺ 348, exp (M+H)⁺ 348.

### Example IVa-22

### (cis-1,5)-5-Hydroxy-1,3,3-trimethyl-cyclohexanecarboxylic acid(4-chloro-3-trifluoromethyl-phenyl)-amide

The title compound was prepared in analogy to example IVa-1 in Synthetic route 14 by using 4-amino-2-trifluoromethyl-benzonitrile instead of aniline. ¹H NMR (d₄-MeOD, 400 MHz), 8.27 (d, 1H, J=2.0 Hz), 8.07 (dd, 1H, J₁=8.4 Hz, J₂=2.0 Hz), 7.92 (d, 1H, J=8.4 Hz), 4.08-4.05 (m, 1H), 1.89-1.87 (m, 2H), 1.74-1.64 (m, 3H), 1.41 (s, 3H), 1.33-1.24 (m, 1H), 1.08 (s, 3H), 1.02 (s, 3H). MS (ESI): calc'd (M+H)⁺ 355, exp (M+H)⁺ 355.

### Example IVb-1

### (cis-1,5)-5-Amino-1,3,3-trimethyl-cyclohexanecarboxylic acid(3-chloro-phenyl)-amide

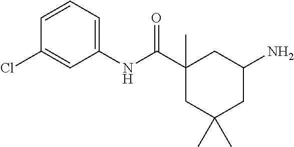

The title compound was prepared by the method as shown in Synthetic route 15.

### Synthetic Route 15 to Example IVb-1

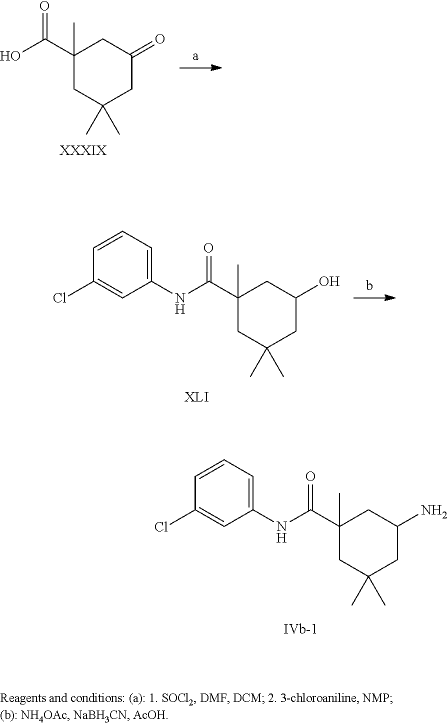

Intermediate XXXIX was treated with thionyl chloride and coupled with 3-chloroaniline to give amide XLI. To a solution of XLI (200 mg, 0.68 mmol) and 1.0 g of ammonium formate in 20 mL of methanol, was added NaBH₃CN (30 mg, 0.5 mmol). The reaction mixture was stirred at r.t. overnight before concentrated in vacuo. The residue was dissolved in water and basified by NaOH (2N) to pH>13. The aqueous solution was extracted with DCM. The organic phase was washed with brine, dried over Na₂SO₄ and concentrated. HPLC separation of the residue gave the target compound IVb-1. ¹H NMR (CDCl₃, 400 MHz), 8.52 (bs, 1H), 7.66 (t, 1H, J=2.0 Hz), 7.41 (d, 1H, J=8.0 Hz), 7.25 (t, 1H, J=8.0 Hz), 7.10 (d, 1H, J=8.0 Hz), 3.27-3.23 (m, 1H), 1.99-1.91 (m, 1H), 1.69-1.64 (m, 3H), 1.50-1.46 (m, 1H), 1.40 (s, 3H), 1.15-1.03 (m, 4H), 0.98 (s, 3H). MS (ESI): calc'd (M+H)⁺ 295, exp (M+H)⁺ 295.

### Example IVb-2

### (cis-1,5)-5-Acetylamino-1,3,3-trimethyl-cyclohexanecarboxylic acid(3-chloro-phenyl)-amide

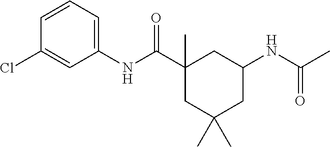

The title compound was prepared by treating IVa-1 with acetyl chloride and NMP at r.t. ¹H NMR (CDCl₃, 400 MHz), 7.81 (bs, 1H), 7.66 (t, 1H, J=2.0 Hz), 7.39 (d, 1H, J=8.0 Hz), 7.25 (t, 1H, J=8.0 Hz), 7.10 (d, 1H, J=8.0 Hz), 6.71 (bs, 1H), 4.17-4.13 (m, 1H), 2.03 (s, 3H), 1.99-1.91 (m, 1H), 1.78-1.72 (m, 2H), 1.57-1.52 (m, 2H), 1.49-1.43 (m, 4H), 1.06 (s, 3H), 0.98 (s, 3H). MS (ESI): calc'd (M+H)⁺ 337, exp (M+H)⁺ 337.

### Example IVb-3

### (cis-1,5)-Diacetylamino-1,3,3-trimethyl-cyclohexanecarboxylic acid(3-chloro-phenyl)-amide

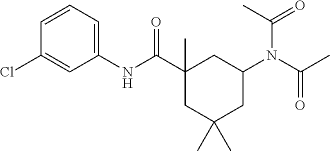

The title compound was prepared by treating IVa-1 with excess acetyl chloride and NMP under refluxing conditions. ¹H NMR (CDCl₃, 400 MHz), 7.81 (bs, 1H), 7.66 (t, 1H, J=2.0 Hz), 7.39 (d, 1H, J=8.0 Hz), 7.25 (t, 1H, J=8.0 Hz), 7.10 (d, 1H, J=8.0 Hz), 4.17-4.13 (m, 1H), 2.41 (s, 6H), 1.99-1.91 (m, 1H), 1.78-1.72 (m, 2H), 1.57-1.52 (m, 2H), 1.49-1.43 (m, 4H), 1.06 (s, 3H), 0.98 (s, 3H). MS: calc'd (M+H)⁺ 379, exp (M+H)⁺ 379.

### Example IVb-4

### (cis-1,5)-5-Methanesulfonylamino-1,3,3-trimethyl-cyclohexanecarboxylic acid(3-chloro-phenyl)-amide

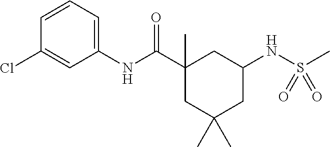

The title compound was prepared by treating IVa-1 with sulfonyl chloride and NMP at r.t. ¹H NMR (CDCl₃, 400 MHz), 7.66 (t, 1H, J=2.0 Hz), 7.42 (bs, 1H), 7.39 (d, 1H, J=8.0 Hz), 7.25 (t, 1H, J=8.0 Hz), 7.10 (d, 1H, J=6.4 Hz), 5.22 (bs, 1H), 3.75-3.73 (m, 1H), 3.07 (s, 3H), 2.03-1.97 (m, 1H), 1.90-1.86 (m, 1H), 1.70-1.66 (m, 2H), 1.54-1.50 (m, 1H), 1.43-1.39 (m, 4H), 1.11 (s, 3H), 1.03 (s, 3H). MS (ESI): calc'd (M+H)⁺ 373, exp (M+H)⁺ 373.

### Biological Examples

### Example V

Cells and viruses. Madin-Darby canine kidney cell (MDCK) was purchased from American type culture collection (ATCC) and was maintained in minimal essential medium (MEM) containing 10% fetal bovine serum and antibiotics. Influenza A/Weiss/43 (H1N1), A/PR/8/34 (H1N1), and A/Hongkong/8/68 (H3N2) were purchased from ATCC and propagated in 10-day-old embryonated chicken eggs at 37° C. Virus was harvested 48 h after inoculation as pooled allantoic fluid. After a brief centrifugation (3,000 rpm at room temperature for 20 min) and virus titer measurement by a hemagglutination test, virus was aliquoted and stored at a −80° C. freezer.

Viral cytopathic effect (CPE) assay: To measure anti-influenza activity of compounds, MDCK cells were seeded into 96-well plates at a density of 5,000 cells per well. Next day, compounds were serially half-log diluted with Gibco SFM containing trypsin. Compounds and 50 pfu of virus were added into corresponding wells to make m.o.i at 0.01 and a final trypsin concentration of 2.5 μg/ml. The testing plates also contained medium control, cell control, virus control, and compound toxicity control. After a 3-day treatment, cell viability was measured with a MTT (3-(4,5-dimethylthiazol-2-yl)-2,5-diphenyltetrazolium bromide) method. Briefly, 20 μl of MTT diluted in culture medium was added into each wells and incubated at 37° C. for 4 hours. Reduced MTT (formazan) was extracted with acidic isopropanol and absorbance at wavelengths of 570 nm and 630 nm (OD₅₇₀ and OD₆₃₀) was read on a microtiter plate reader. After subtraction of background OD values, dose response curves of half-log concentration vs. percent protection were generated, on which half maximal effective concentration (EC₅₀) and half maximal toxic concentration (CC₅₀) were calculated.

### Example VI

Results of H1N1_CPE_EC₅₀ (μM) assay and Cytotoxicy IC₅₀ (μM) are given in Table 1.

### Example A

A compound of formula (I) can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example B

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula (I) 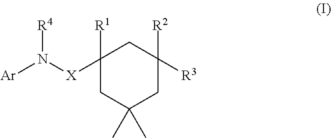
R¹ is hydrogen, C₁₋₆ alkyl, or trifluoromethyl;
R²/R³ are hydrogen, halogen, OR¹⁰, or NR¹¹R¹²
R⁴ is hydrogen, C₁₋₆ alkyl, or trifluoromethyl;
X is —CH₂—, or carbonyl;
Ar is selected from 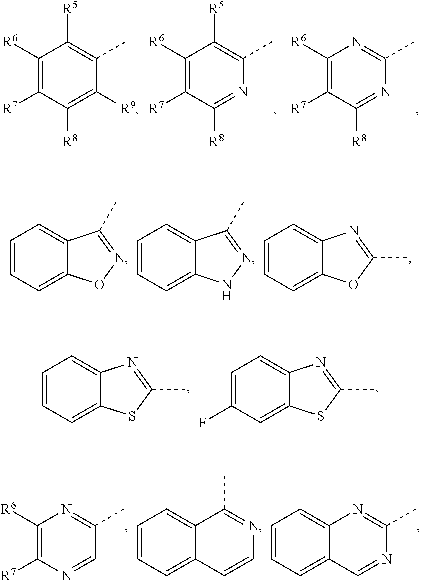
Wherein
R⁵/R⁹ is hydrogen, halogen, trifluoromethyl, or C₁₋₆ alkyl;
R⁶/R⁸ is hydrogen, halogen, trifluoromethyl, trifluoromethoxy, C₁₋₆ alkoxy, cyano, C₁₋₆ alkyl, —C(O)—NH₂, —S(O)₂—NH₂, or —S(O)₂—C₁₋₆ alkyl;
R⁷ is hydrogen, halogen, C₁₋₆ alkyl, cyano, C₁₋₆ alkoxy, or —S(O)₂—NH₂
R¹⁰ is hydrogen, C₁₋₆ alkyl, carbonyl-C₁₋₆ alkyl, or trifluoromethyl;
R¹¹ or R¹² is hydrogen, C₁₋₆ alkyl, carbonyl-C₁₋₆ alkyl, or sulfonyl;
provided that R¹, R², R³, R⁴, R⁵, R⁶, R⁷, R⁸ and R⁹ are not hydrogen simultaneously; and
pharmaceutically acceptable salt and stereoisomers thereof.

2. A compound according to claim 1 or a pharmaceutically acceptable salt thereof, wherein
R¹ is hydrogen or C₁₋₆ alkyl;
R²/R³ are hydrogen, halogen, OR¹⁰, or NR¹¹R¹²
R⁴ is hydrogen or C₁₋₆ alkyl;
X is —CH₂—, or carbonyl;
Ar is selected from 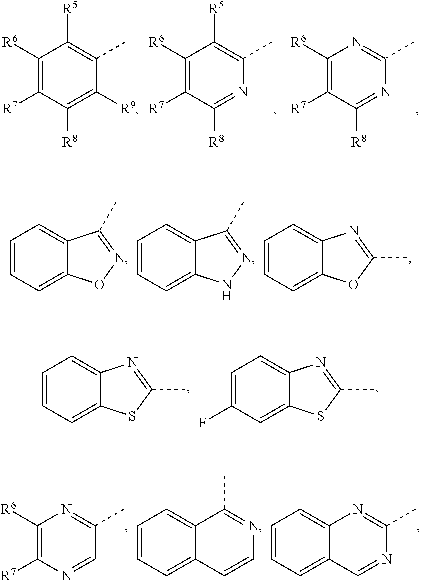
Wherein
R⁵/R⁹ is hydrogen, halogen, trifluoromethyl, or C₁₋₆ alkyl;
R⁶/R⁸ is hydrogen, halogen, trifluoromethyl, trifluoromethoxy, C₁₋₆alkoxy, cyano, C₁₋₆ alkyl, —C(O)—NH₂, —S(O)₂—NH₂, or —S(O)₂—C₁₋₆ alkyl;
R⁷ is hydrogen, halogen, C₁₋₆ alkyl, cyano, C₁₋₆alkoxy, or —S(O)₂—NH₂
R¹⁰ is hydrogen, C₁₋₆ alkyl, carbonyl-C₁₋₆ alkyl, or trifluoromethyl;
R¹¹ or R¹² is hydrogen, C₁₋₆ alkyl, carbonyl-C₁₋₆ alkyl, or sulfonyl;
provided that R¹, R², R³, R⁴, R⁵, R⁶, R⁷, R⁸ and R⁹ are not hydrogen simultaneously and compound with two chiral center is in cis configuration.

3. The compound of claim 1, wherein R¹ is C₁₋₆ alkyl.

4. The compound of claim 1, wherein R¹ is methyl.

5. The compound of claim 1, wherein R² or R³ is OR¹⁰.

6. The compound of claim 5, wherein R² or R³ is hydroxy.

7. The compound of claim 1, wherein R⁴ is hydrogen.

8. The compound of claim 7, wherein R² or R³ is hydroxyl.

9. The compound of claim 1, wherein Ar is selected from 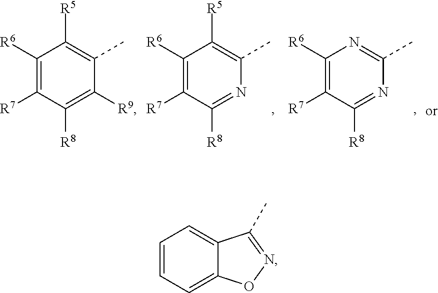
wherein R⁵/R⁹ is hydrogen or halogen;
R⁶/R⁸ is halogen, trifluoromethyl, trifluoromethoxy, cyano, —S(O)₂—NH₂, or —S(O)₂—C₁₋₆alkyl;
R⁷ is hydrogen, cyano, or halogen.

10. The compound of claim 9, wherein R² or R³ is hydroxyl and R⁴ is hydrogen.

11. The compound of claim 10, wherein Ar is 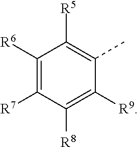
R⁵/R⁹ is hydrogen, chloro, or fluoro;
R⁶/R⁸ is halogen, trifluoromethyl, cyano, —S(O)₂—NH₂, or —S(O)₂-methyl;
R⁷ is hydrogen, chloro, or fluoro.

12. The compound of claim 1, wherein X is —CH₂—.

13. The compound of claim 12, wherein
R² or R³ is hydroxyl;
R⁴ is hydrogen;
Ar is selected from 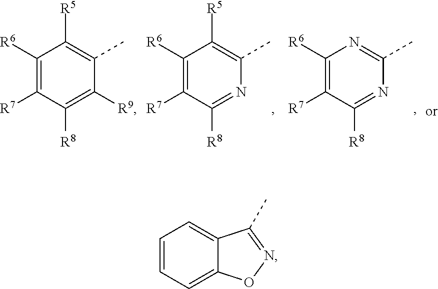
wherein R⁵/R⁹ is hydrogen or halogen;
R⁶/R⁸ is halogen, trifluoromethyl, trifluoromethoxy, cyano, —S(O)₂—NH₂, or —S(O)₂—C₁₋₆alkyl;
R⁷ is hydrogen, cyano, or halogen.

14. The compound of claim 13, wherein
Ar is 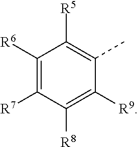
R⁵/R⁹ is hydrogen, chloro, or fluoro;
R⁶/R⁸ is halogen, trifluoromethyl, cyano, —S(O)₂—NH₂, or —S(O)₂-methyl;
R⁷ is hydrogen, chloro, or fluoro.

15. A compound selected from the group consisting of:
(cis-1,5)-3,3,5-Trimethyl-5-phenylaminomethyl-cyclohexanol,
(cis-1,3)-3-[(2-Chloro-phenylamino)-methyl]-3,5,5-trimethyl-cyclohexanol,
(cis-1,3)-3-[(3-Chloro-phenylamino)-methyl]-3,5,5-trimethyl-cyclohexanol,
(cis-1,3)-3-[(4-Chloro-phenylamino)-methyl]-3,5,5-trimethyl-cyclohexanol,
(cis-1,5)-3,3,5-Trimethyl-5-[(2-trifluoromethyl-phenylamino)-methyl]-cyclohexanol,
(cis-1,5)-3,3,5-Trimethyl-5-[(3-trifluoromethyl-phenylamino)-methyl]-cyclohexanol,
(cis-1,5)-3,3,5-Trimethyl-5-(p-tolylamino-methyl)-cyclohexanol,
(cis-1,3)-3-[(3-Methoxy-phenylamino)-methyl]-3,5,5-trimethyl-cyclohexanol,
3-[((cis-1,5)-5-Hydroxy-1,3,3-trimethyl-cyclohexylmethyl)-amino]-benzonitrile,
4-[((cis-1,5)-5-Hydroxy-1,3,3-trimethyl-cyclohexylmethyl)-amino]-benzonitrile,
(cis-1,3)-3-[(3-Isopropoxy-phenylamino)-methyl]-3,5,5-trimethyl-cyclohexanol,
(cis-1,3)-3-[(3-Isopropyl-phenylamino)-methyl]-3,5,5-trimethyl-cyclohexanol,
3-[((cis-1,5)-5-Hydroxy-1,3,3-trimethyl-cyclohexylmethyl)-amino]-benzamide,
(cis-1,3)-3-[(3-Methanesulfonyl-phenylamino)-methyl]-3,5,5-trimethyl-cyclohexanol,
3-[((cis-1,5)-5-Hydroxy-1,3,3-trimethyl-cyclohexylmethyl)-amino]-benzenesulfonamide,
(cis-1,3)-3-[(3-Chloro-5-fluoro-phenylamino)-methyl]-3,5,5-trimethyl-cyclohexanol,
(cis-1,3)-3-[(3-Fluoro-5-trifluoromethyl-phenylamino)-methyl]-3,5,5-trimethyl-cyclohexanol,
(cis-1,3)-3-[(3-Chloro-5-methyl-phenylamino)-methyl]-3,5,5-trimethyl-cyclohexanol,
(cis-1,3)-3-[(3,5-Dichloro-phenylamino)-methyl]-3,5,5-trimethyl-cyclohexanol,
(cis-1,3)-3-[(3-Chloro-5-trifluoromethyl-phenylamino)-methyl]-3,5,5-trimethyl-cyclohexanol,
(cis-1,3)-3-[(3-Bromo-5-trifluoromethyl-phenylamino)-methyl]-3,5,5-trimethyl-cyclohexanol,
3-Fluoro-5-[((cis-1,5)-5-hydroxy-1,3,3-trimethyl-cyclohexylmethyl)-amino]-benzonitrile,
3-Chloro-5-[((cis-1,5)-5-hydroxy-1,3,3-trimethyl-cyclohexylmethyl)-amino]-benzonitrile,
3-[((cis-1,5)-5-Hydroxy-1,3,3-trimethyl-cyclohexylmethyl)-amino]-5-trifluoromethyl-benzonitrile,
(cis-1,3)-3-[(3,5-Bis-trifluoromethyl-phenylamino)-methyl]-3,5,5-trimethyl-cyclohexanol,
(cis-1,3)-3-[(2,3-Dichloro-phenylamino)-methyl]-3,5,5-trimethyl-cyclohexanol,
(cis-1,3)-3-[(3,4-Dichloro-phenylamino)-methyl]-3,5,5-trimethyl-cyclohexanol,
2-Fluoro-5-[((cis-1,5)-5-hydroxy-1,3,3-trimethyl-cyclohexylmethyl)-amino]-benzonitrile,
2-Chloro-4-[((cis-1,5)-5-hydroxy-1,3,3-trimethyl-cyclohexylmethyl)-amino]-benzonitrile,
2-Chloro-5-[((cis-1,5)-5-hydroxy-1,3,3-trimethyl-cyclohexylmethyl)-amino]-benzonitrile,
4-[((cis-1,5)-5-Hydroxy-1,3,3-trimethyl-cyclohexylmethyl)-amino]-phthalonitrile,
(cis-1,3)-3-[(4-Chloro-3-trifluoromethyl-phenylamino)-methyl]-3,5,5-trimethyl-cyclohexanol,
4-[((cis-1,5)-5-Hydroxy-1,3,3-trimethyl-cyclohexylmethyl)-amino]-2-trifluoromethyl-benzonitrile,
2-Chloro-5-[((cis-1,5)-5-hydroxy-1,3,3-trimethyl-cyclohexylmethyl)-amino]-benzenesulfonamide,
3-Chloro-5-[((cis-1,5)-5-hydroxy-1,3,3-trimethyl-cyclohexylmethyl)-amino]-benzenesulfonamide,
3-[((cis-1,5)-5-Hydroxy-1,3,3-trimethyl-cyclohexylmethyl)-amino]-5-trifluoromethyl-benzenesulfonamide,
3-Fluoro-5-[((cis-1,5)-5-hydroxy-1,3,3-trimethyl-cyclohexylmethyl)-amino]-benzenesulfonamide,
5-[((cis-1,5)-5-Hydroxy-1,3,3-trimethyl-cyclohexylmethyl)-amino]-2-methyl-benzenesulfonamide,
5-[((cis-1,5)-5-Hydroxy-1,3,3-trimethyl-cyclohexylmethyl)-amino]-2-methoxy-benzenesulfonamide,
4-[((cis-1,5)-5-Hydroxy-1,3,3-trimethyl-cyclohexylmethyl)-amino]-2-trifluoromethoxy-benzenesulfonamide,
(cis-1,3)-3-{[(3-Chloro-phenyl)-methyl-amino]-methyl}-3,5,5-trimethyl-cyclohexanol,
(3-Fluoro-5-trifluoromethyl-phenyl)-(1,3,3-trimethyl-cyclohexylmethyl)-amine,
Acetic acid (cis-1,5)-3,3,5-trimethyl-5-phenylaminomethyl-cyclohexyl ester,
(3,3-Difluoro-1,5,5-trimethyl-cyclohexylmethyl)-(3-fluoro-5-trifluoromethyl-phenyl)-amine,
(3-Bromo-5-trifluoromethyl-phenyl)-(3,3-difluoro-1,5,5-trimethyl-cyclohexylmethyl)-amine,
(3-Fluoro-5-trifluoromethyl-phenyl)-((cis-1,5)-5-methoxy-1,3,3-trimethyl-cyclohexylmethyl)-amine,
(cis-1,3)-3-[(3-Fluoro-5-trifluoromethyl-phenylamino)-methyl]-1,3,5,5-tetramethyl-cyclohexanol,
((cis-1,5)-5-Dimethylamino-1,3,3-trimethyl-cyclohexylmethyl)-(3-fluoro-5-trifluoromethyl-phenyl)-amine,
(cis-1,5)-5-[(3-Fluoro-5-trifluoromethyl-phenylamino)-methyl]-3,3-dimethyl-cyclohexanol,
(trans-1,5)-5-[(3-fluoro-5-trifluoromethyl-phenylamino)-methyl]-3,3-dimethyl-cyclohexanol,
(cis-1,5)-3,3,5-Trimethyl-5-(pyridin-2-ylaminomethyl)-cyclohexanol,
(cis-1,3)-3-[(5-Bromo-pyridin-2-ylamino)-methyl]-3,5,5-trimethyl-cyclohexanol,
(cis-1,5)-3,3,5-Trimethyl-5-[(6-trifluoromethyl-pyridin-2-ylamino)-methyl]-cyclohexanol,
(cis-1,3)-3-[(4-Chloro-pyridin-2-ylamino)-methyl]-3,5,5-trimethyl-cyclohexanol,
(cis-1,5)-3,3,5-Trimethyl-5-[(3-trifluoromethyl-pyridin-2-ylamino)-methyl]-cyclohexanol,
(cis-1,5)-3,3,5-Trimethyl-5-[(5-trifluoromethyl-pyridin-2-ylamino)-methyl]-cyclohexanol,
6-[((cis-1,5)-5-Hydroxy-1,3,3-trimethyl-cyclohexylmethyl)-amino]-pyridine-2-sulfonic acid amide,
2-[((cis-1,5)-5-Hydroxy-1,3,3-trimethyl-cyclohexylmethyl)-amino]-pyridine-4-sulfonic acid amide,
(cis-1,5)-3,3,5-Trimethyl-5-[(4-methyl-pyrimidin-2-ylamino)-methyl]-cyclohexanol,
(cis-1,5)-3,3,5-Trimethyl-5-[(4-trifluoromethyl-pyrimidin-2-ylamino)-methyl]-cyclohexanol,
(cis-1,3)-3-[(4-Methoxy-pyrimidin-2-ylamino)-methyl]-3,5,5-trimethyl-cyclohexanol,
(cis-1,3)-3-[(4,6-Dimethoxy-pyrimidin-2-ylamino)-methyl]-3,5,5-trimethyl-cyclohexanol,
(cis-1,3)-3-[(4,6-Dimethyl-pyrimidin-2-ylamino)-methyl]-3,5,5-trimethyl-cyclohexanol,
(cis-1,3)-3-[(4-Chloro-5-methoxy-pyrimidin-2-ylamino)-methyl]-3,5,5-trimethyl-cyclohexanol,
(cis-1,3)-3-[(6-Chloro-pyrazin-2-ylamino)-methyl]-3,5,5-trimethyl-cyclohexanol,
(cis-1,5)-3,3,5-Trimethyl-5-[(6-methyl-pyrazin-2-ylamino)-methyl]-cyclohexanol,
(cis-1,3)-3-[(6-Methoxy-pyrazin-2-ylamino)-methyl]-3,5,5-trimethyl-cyclohexanol,
(cis-1,3)-3-(Benzoxazol-2-ylaminomethyl)-3,5,5-trimethyl-cyclohexanol,
Benzoxazol-2-yl-(3,3-difluoro-1,5,5-trimethyl-cyclohexylmethyl)-amine,
Benzoxazol-2-yl-((cis-1,5)-5-methoxy-1,3,3-trimethyl-cyclohexylmethyl)-amine,
(cis-1,3)-3-(Benzothiazol-2-ylaminomethyl)-3,5,5-trimethyl-cyclohexanol,
(cis-1,3)-3-[(6-Fluoro-benzothiazol-2-ylamino)-methyl]-3,5,5-trimethyl-cyclohexanol,
(cis-1,3)-3-(Benzo[d]isoxazol-3-ylaminomethyl)-3,5,5-trimethyl-cyclohexanol,
(cis-1,3)-3-[(1H-Indazol-3-ylamino)-methyl]-3,5,5-trimethyl-cyclohexanol,
(cis-1,5)-3,3,5-Trimethyl-5-(quinazolin-2-ylaminomethyl)-cyclohexanol,
(cis-1,3)-3-(Isoquinolin-1-ylaminomethyl)-3,5,5-trimethyl-cyclohexanol,
(cis-1,5)-5-Hydroxy-1,3,3-trimethyl-cyclohexanecarboxylic acid phenylamide,
(cis-1,5)-5-Hydroxy-1,3,3-trimethyl-cyclohexanecarboxylic acid(4-fluoro-phenyl)-amide,
(cis-1,5)-5-Hydroxy-1,3,3-trimethyl-cyclohexanecarboxylic acid(3-chloro-phenyl)-amide,
(cis-1,5)-5-Hydroxy-1,3,3-trimethyl-cyclohexanecarboxylic acid(3-trifluoromethyl-phenyl)-amide,
(cis-1,5)-5-Hydroxy-1,3,3-trimethyl-cyclohexanecarboxylic acid(3-isopropyl-phenyl)-amide,
(cis-1,5)-5-Hydroxy-1,3,3-trimethyl-cyclohexanecarboxylic acid(3-tert-butyl-phenyl)-amide,
(cis-1,5)-5-Hydroxy-1,3,3-trimethyl-cyclohexanecarboxylic acid(3-ethoxy-phenyl)-amide,
(cis-1,5)-5-Hydroxy-1,3,3-trimethyl-cyclohexanecarboxylic acid(3-trifluoromethoxy-phenyl)-amide,
(cis-1,5)-5-Hydroxy-1,3,3-trimethyl-cyclohexanecarboxylic acid(3-methanesulfonyl-phenyl)-amide,
(cis-1,5)-5-Hydroxy-1,3,3-trimethyl-cyclohexanecarboxylic acid(3-sulfamoyl-phenyl)-amide,
(cis-1,5)-5-Hydroxy-1,3,3-trimethyl-cyclohexanecarboxylic acid(3,5-difluoro-phenyl)-amide,
(cis-1,5)-5-Hydroxy-1,3,3-trimethyl-cyclohexanecarboxylic acid(3-chloro-5-fluoro-phenyl)amide,
(cis-1,5)-5-Hydroxy-1,3,3-trimethyl-cyclohexanecarboxylic acid(3-fluoro-5-trifluoromethyl-phenyl)-amide,
(cis-1,5)-5-Hydroxy-1,3,3-trimethyl-cyclohexanecarboxylic acid(3-chloro-5-trifluoromethyl-phenyl)-amide,
(cis-1,5)-5-Hydroxy-1,3,3-trimethyl-cyclohexanecarboxylic acid(3-bromo-5-trifluoromethyl-phenyl)-amide,
(cis-1,5)-5-Hydroxy-1,3,3-trimethyl-cyclohexanecarboxylic acid(3-methoxy-5-trifluoromethyl-phenyl)-amide,
(cis-1,5)-5-Hydroxy-1,3,3-trimethyl-cyclohexanecarboxylic acid(3,5-dichloro-phenyl)-amide,
(cis-1,5)-5-Hydroxy-1,3,3-trimethyl-cyclohexanecarboxylic acid(3,5-dimethoxy-phenyl)-amide,
(cis-1,5)-5-Hydroxy-1,3,3-trimethyl-cyclohexanecarboxylic acid(4-chloro-3-fluoro-phenyl)amide,
(cis-1,5)-5-Hydroxy-1,3,3-trimethyl-cyclohexanecarboxylic acid(3,4-dichloro-phenyl)-amide,
(cis-1,5)-5-Hydroxy-1,3,3-trimethyl-cyclohexanecarboxylic acid(4-fluoro-3-trifluoromethyl-phenyl)-amide,
(cis-1,5)-5-Hydroxy-1,3,3-trimethyl-cyclohexanecarboxylic acid(4-chloro-3-trifluoromethyl-phenyl)-amide,
(cis-1,5)-5-Hydroxy-1,3,3-trimethyl-cyclohexanecarboxylic acid(4-methoxy-3-trifluoromethyl-phenyl)-amide,
(cis-1,5)-5-Hydroxy-1,3,3-trimethyl-cyclohexanecarboxylic acid(4-cyano-3-trifluoromethyl-phenyl)-amide,
(cis-1,5)-5-Amino-1,3,3-trimethyl-cyclohexanecarboxylic acid(3-chloro-phenyl)-amide,
(cis-1,5)-5-Acetylamino-1,3,3-trimethyl-cyclohexanecarboxylic acid(3-chloro-phenyl)-amide,
(cis-1,5)-Diacetylamino-1,3,3-trimethyl-cyclohexanecarboxylic acid(3-chloro-phenyl)-amide, and
(cis-1,5)-5-Methanesulfonylamino-1,3,3-trimethyl-cyclohexanecarboxylic acid(3-chloro-phenyl)-amide.

16. A compound selected from the group consisting of:
(cis-1,5)-3,3,5-Trimethyl-5-phenylaminomethyl-cyclohexanol,
(cis-1,3)-3-[(2-Chloro-phenylamino)-methyl]-3,5,5-trimethyl-cyclohexanol,
(cis-1,3)-3-[(3-Chloro-phenylamino)-methyl]-3,5,5-trimethyl-cyclohexanol,
(cis-1,3)-3-[(4-Chloro-phenylamino)-methyl]-3,5,5-trimethyl-cyclohexanol,
(cis-1,5)-3,3,5-Trimethyl-5-[(2-trifluoromethyl-phenylamino)-methyl]-cyclohexanol,
(cis-1,5)-3,3,5-Trimethyl-5-[(3-trifluoromethyl-phenylamino)-methyl]-cyclohexanol,
(cis-1,3)-3-[(3-Methoxy-phenylamino)-methyl]-3,5,5-trimethyl-cyclohexanol,
3-[((cis-1,5)-5-Hydroxy-1,3,3-trimethyl-cyclohexylmethyl)-amino]-benzonitrile,
(cis-1,3)-3-[(3-Isopropyl-phenylamino)-methyl]-3,5,5-trimethyl-cyclohexanol,
(cis-1,3)-3-[(3-Methanesulfonyl-phenylamino)-methyl]-3,5,5-trimethyl-cyclohexanol,
3-[((cis-1,5)-5-Hydroxy-1,3,3-trimethyl-cyclohexylmethyl)-amino]-benzenesulfonamide,
(cis-1,3)-3-[(3-Chloro-5-fluoro-phenylamino)-methyl]-3,5,5-trimethyl-cyclohexanol,
(cis-1,3)-3-[(3-Fluoro-5-trifluoromethyl-phenylamino)-methyl]-3,5,5-trimethyl-cyclohexanol,
(cis-1,3)-3-[(3-Chloro-5-methyl-phenylamino)-methyl]-3,5,5-trimethyl-cyclohexanol,
(cis-1,3)-3-[(3,5-Dichloro-phenylamino)-methyl]-3,5,5-trimethyl-cyclohexanol,
(cis-1,3)-3-[(3-Chloro-5-trifluoromethyl-phenylamino)-methyl]-3,5,5-trimethyl-cyclohexanol,
(cis-1,3)-3-[(3-Bromo-5-trifluoromethyl-phenylamino)-methyl]-3,5,5-trimethyl-cyclohexanol,
3-Fluoro-5-[((cis-1,5)-5-hydroxy-1,3,3-trimethyl-cyclohexylmethyl)-amino]-benzonitrile,
3-Chloro-5-[((cis-1,5)-5-hydroxy-1,3,3-trimethyl-cyclohexylmethyl)-amino]-benzonitrile,
3-[((cis-1,5)-5-Hydroxy-1,3,3-trimethyl-cyclohexylmethyl)-amino]-5-trifluoromethyl-benzonitrile,
(cis-1,3)-3-[(3,5-Bis-trifluoromethyl-phenylamino)-methyl]-3,5,5-trimethyl-cyclohexanol,
(cis-1,3)-3-[(3,4-Dichloro-phenylamino)-methyl]-3,5,5-trimethyl-cyclohexanol,
2-Fluoro-5-[((cis-1,5)-5-hydroxy-1,3,3-trimethyl-cyclohexylmethyl)-amino]-benzonitrile,
2-Chloro-4-[((cis-1,5)-5-hydroxy-1,3,3-trimethyl-cyclohexylmethyl)-amino]-benzonitrile,
2-Chloro-5-[((cis-1,5)-5-hydroxy-1,3,3-trimethyl-cyclohexylmethyl)-amino]-benzonitrile,
(cis-1,3)-3-[(4-Chloro-3-trifluoromethyl-phenylamino)-methyl]-3,5,5-trimethyl-cyclohexanol,
4-[((cis-1,5)-5-Hydroxy-1,3,3-trimethyl-cyclohexylmethyl)-amino]-2-trifluoromethyl-benzonitrile,
2-Chloro-5-[((cis-1,5)-5-hydroxy-1,3,3-trimethyl-cyclohexylmethyl)-amino]-benzenesulfonamide,
3-Chloro-5-[((cis-1,5)-5-hydroxy-1,3,3-trimethyl-cyclohexylmethyl)-amino]-benzenesulfonamide,
3-Fluoro-5-[((cis-1,5)-5-hydroxy-1,3,3-trimethyl-cyclohexylmethyl)-amino]-benzenesulfonamide,
(cis-1,3)-3-{[(3-Chloro-phenyl)-methyl-amino]-methyl}-3,5,5-trimethyl-cyclohexanol,
(3-Fluoro-5-trifluoromethyl-phenyl)-(1,3,3-trimethyl-cyclohexylmethyl)-amine,
Acetic acid (cis-1,5)-3,3,5-trimethyl-5-phenylaminomethyl-cyclohexyl ester,
(3,3-Difluoro-1,5,5-trimethyl-cyclohexylmethyl)-(3-fluoro-5-trifluoromethyl-phenyl)-amine,
(3-Fluoro-5-trifluoromethyl-phenyl)-((cis-1,5)-5-methoxy-1,3,3-trimethyl-cyclohexylmethyl)-amine,
(cis-1,3)-3-[(3-fluoro-5-trifluoromethyl-phenylamino)-methyl]-1,3,5,5-tetramethyl-cyclohexanol,
((cis-1,5)-5-Dimethylamino-1,3,3-trimethyl-cyclohexylmethyl)-(3-fluoro-5-trifluoromethyl-phenyl)-amine,
(cis-1,5)-5-[(3-fluoro-5-trifluoromethyl-phenylamino)-methyl]-3,3-dimethyl-cyclohexanol,
(trans-1,5)-5-[(3-fluoro-5-trifluoromethyl-phenylamino)-methyl]-3,3-dimethyl-cyclohexanol,
(cis-1,5)-3,3,5-trimethyl-5-[(6-trifluoromethyl-pyridin-2-ylamino)-methyl]-cyclohexanol,
(cis-1,5)-3,3,5-trimethyl-5-[(3-trifluoromethyl-pyridin-2-ylamino)-methyl]-cyclohexanol,
2-[((cis-1,5)-5-Hydroxy-1,3,3-trimethyl-cyclohexylmethyl)-amino]-pyridine-4-sulfonic acid amide,
(cis-1,5)-3,3,5-trimethyl-5-[(4-trifluoromethyl-pyrimidin-2-ylamino)-methyl]-cyclohexanol,
(cis-1,3)-3-[(4-methoxy-pyrimidin-2-ylamino)-methyl]-3,5,5-trimethyl-cyclohexanol,
(cis-1,3)-3-[(4,6-dimethyl-pyrimidin-2-ylamino)-methyl]-3,5,5-trimethyl-cyclohexanol,
(cis-1,3)-3-[(6-chloro-pyrazin-2-ylamino)-methyl]-3,5,5-trimethyl-cyclohexanol,
(cis-1,3)-3-(benzooxazol-2-ylaminomethyl)-3,5,5-trimethyl-cyclohexanol,
Benzooxazol-2-yl-(3,3-difluoro-1,5,5-trimethyl-cyclohexylmethyl)-amine,
Benzooxazol-2-yl-((cis-1,5)-5-methoxy-1,3,3-trimethyl-cyclohexylmethyl)-amine,
(cis-1,3)-3-(benzothiazol-2-ylaminomethyl)-3,5,5-trimethyl-cyclohexanol,
(cis-1,3)-3-[(6-fluoro-benzothiazol-2-ylamino)-methyl]-3,5,5-trimethyl-cyclohexanol,
(cis-1,3)-3-(benzo[d]isoxazol-3-ylaminomethyl)-3,5,5-trimethyl-cyclohexanol,
(cis-1,3)-3-[(1H-indazol-3-ylamino)-methyl]-3,5,5-trimethyl-cyclohexanol,
(cis-1,5)-3,3,5-trimethyl-5-(quinazolin-2-ylaminomethyl)-cyclohexanol,
(cis-1,3)-3-(Isoquinolin-1-ylaminomethyl)-3,5,5-trimethyl-cyclohexanol,
(cis-1,5)-5-Hydroxy-1,3,3-trimethyl-cyclohexanecarboxylic acid(3-chloro-phenyl)-amide,
(cis-1,5)-5-Hydroxy-1,3,3-trimethyl-cyclohexanecarboxylic acid(3-trifluoromethyl-phenyl)-amide,
(cis-1,5)-5-Hydroxy-1,3,3-trimethyl-cyclohexanecarboxylic acid(3-isopropyl-phenyl)-amide,
(cis-1,5)-5-Hydroxy-1,3,3-trimethyl-cyclohexanecarboxylic acid(3-trifluoromethoxy-phenyl)-amide,
(cis-1,5)-5-Hydroxy-1,3,3-trimethyl-cyclohexanecarboxylic acid(3-sulfamoyl-phenyl)-amide,
(cis-1,5)-5-Hydroxy-1,3,3-trimethyl-cyclohexanecarboxylic acid(3,5-difluoro-phenyl)-amide,
(cis-1,5)-5-Hydroxy-1,3,3-trimethyl-cyclohexanecarboxylic acid(3-chloro-5-fluoro-phenyl)amide,
(cis-1,5)-5-Hydroxy-1,3,3-trimethyl-cyclohexane carboxylic acid(3-fluoro-5-trifluoromethyl-phenyl)-amide,
(cis-1,5)-5-Hydroxy-1,3,3-trimethyl-cyclohexane carboxylic acid(3-chloro-5-trifluoromethyl-phenyl)-amide,
(cis-1,5)-5-Hydroxy-1,3,3-trimethyl-cyclohexanecarboxylic acid(3-bromo-5-trifluoromethyl-phenyl)-amide,
(cis-1,5)-5-Hydroxy-1,3,3-trimethyl-cyclohexanecarboxylic acid(3-methoxy-5-trifluoromethyl-phenyl)-amide,
(cis-1,5)-5-Hydroxy-1,3,3-trimethyl-cyclohexanecarboxylic acid(3,5-dichloro-phenyl)-amide,
(cis-1,5)-5-Hydroxy-1,3,3-trimethyl-cyclohexanecarboxylic acid(4-chloro-3-fluoro-phenyl)amide,
(cis-1,5)-5-Hydroxy-1,3,3-trimethyl-cyclohexanecarboxylic acid(3,4-dichloro-phenyl)-amide,
(cis-1,5)-5-Hydroxy-1,3,3-trimethyl-cyclohexane carboxylic acid(4-fluoro-3-trifluoromethyl-phenyl)-amide,
(cis-1,5)-5-Hydroxy-1,3,3-trimethyl-cyclohexane carboxylic acid(4-chloro-3-trifluoromethyl-phenyl)-amide,
(cis-1,5)-5-Acetylamino-1,3,3-trimethyl-cyclohexanecarboxylic acid(3-chloro-phenyl)-amide,
(cis-1,5)-Diacetylamino-1,3,3-trimethyl-cyclohexanecarboxylic acid(3-chloro-phenyl)-amide, and
(cis-1,5)-5-Methanesulfonylamino-1,3,3-trimethyl-cyclohexanecarboxylic acid(3-chloro-phenyl)-amide.

17. A method for treatment of influenza comprising administering to a subject in need thereof a therapeutically effective amount of a compound of claim 1.

18. A method for prevention of influenza comprising administering to a subject in need thereof a therapeutically effective amount of a compound of claim 1.

19. A method for the treatment or prevention of diseases that are related to HA inhibition comprising administering to a subject in need thereof a therapeutically effective amount of a compound of claim 1.

20. A pharmaceutical composition comprising:
(a) a pharmaceutically acceptable carrier; and
(b) a compound of claim 1.
